(19) United States
(12) Patent Application Publication
(10) Pub. No.: US 2020/0323247 A1
(43) Pub. Date: Oct. 15, 2020

(54) GOOD-TEXTURE HEALTHY BOILED NOODLE

(71) Applicant: SHIMADAYA CORPORATION, Tokyo (JP)

(72) Inventors: Fumiya Nishibori, Tokyo (JP); Kazuyo Shiragami, Tokyo (JP); Mariko Koike, Tokyo (JP); Miho Tomokane, Tokyo (JP); Takahiro Seki, Tokyo (JP); Takuo Ogihara, Gunma (JP); Makoto Ezaki, Tokyo (JP)

(21) Appl. No.: 16/957,574

(22) PCT Filed: Jul. 30, 2018
(86) PCT No.: PCT/JP2018/028437

§ 371 Date: Jun. 24, 2020

(30) Foreign Application Priority Data
Dec. 28, 2017 (JP) 2017-253530

(51) Int. Cl. A23L 7/113; A21D 2/18; A21D 8/02; A23L 5/10; A23L 33/00; A61K 33/06; A61K 31/734; A61K 9/00; A61P 3/10

(52) U.S. Cl. CPC A23L 7/113; A21D 2/183; A21D 8/02; A23L 5/13; A23V 2002/00; A61K 33/06; A61K 31/734; A61K 9/0056; A61P 3/10; A23L 33/40

(57) ABSTRACT

An object of the present invention is to provide good-texture healthy boiled noodles or raw noodles having an effective health function of calcium alginate and excellent in texture by imparting a health function of calcium alginate to noodles, and in addition, without impairing original texture of noodles, and in order to solve the object, in a method for producing boiled noodles or raw noodles employing, as noodle making steps in a noodle production method, (A) a mixing and kneading step of kneading a dough by adding kneading water to a powder raw material for noodle production obtained through a mixture of raw materials containing wheat flour; and (B) a noodle making step of forming the dough into noodle strings, a 270-mesh pass pulverized calcium alginate powder is mixed in the powder raw material for noodle production in a ratio of 4 to 8 parts by mass based on 100 parts by mass of the powder raw material for noodle production, and thus, healthy boiled noodles or raw noodles with the health function of calcium alginate and retaining a good texture of noodles are produced. The present invention provides good-texture healthy boiled noodles or raw noodles with original texture of noodles retained, effectively retaining the health function such as an effect of inhibiting postprandial blood glucose level increase, and in addition, without causing, in production of the noodles, deterioration of viscoelasticity and the like of noodle tissue and deterioration of texture otherwise caused in adding calcium alginate.

### TECHNICAL FIELD

The present invention relates to provision of good-texture healthy boiled noodles excellent in texture by imparting a health function of calcium alginate to noodles, and in addition, without impairing original texture of boiled noodles. Furthermore, the present invention relates to provision of good-texture healthy raw noodles for boiled cooking excellent in texture by imparting a health function of calcium alginate to noodles, and in addition, without impairing original texture of noodles. This application claims the benefit of priority of Japanese Patent Application No. 2017-253530, filed on Dec. 28, 2017, the entire contents of which are incorporated herein by reference.

### BACKGROUND ART

The primary meaning of foodstuff is originally that a taste, a texture and a flavor are appreciated by ingesting the food, and thus, components necessary for the body are supplied, but in recent years, owing to rise in health consciousness, consciousness to demand a health function of food itself is increasing. For example, one of various factors of lifestyle-related diseases in these days is a problem of diabetes, and since the number of diabetes patients and persons suspected of having diabetes keeps on increasing currently in Japan, various examinations are being made also for countermeasures against such diseases. Today's eating habits, lack of exercise, stress and the like are deeply involved in the onset of type II diabetes, and as a measure against it, diet therapy and exercise therapy are regarded as effective methods for diabetes treatment. Studies on prevention of diabetes in the field of food science are made for purposes of restraining hyperglycemia by inhibiting and delaying degradation and absorption of carbohydrate, and food and drink are also demanded to have such a health-promoting function. Besides, from the viewpoint of diet, not a few consumers want to avoid food causing rapid increase of blood glucose level.

As an active ingredient in providing health food capable of contributing to prevention and treatment of lifestyle-related diseases and the like by imparting a health function to food or the like, calcium alginate is known. Alginic acid is a natural polysaccharide contained in brown algae such as kelp and wakame, and alginic acid and alginates are widely used in the field of food as a thickener, a stabilizer and a gelling agent. Calcium alginate is a component also used as various additives for food, and in recent years, this component is reported to be used as a health function component. For example, Non-patent Document 1 reports “Examination Test of Influence of Ingestion of Calcium Alginate-containing Food on Leg Swelling in Adult Females” (pp. 102-108) and “Influence of Single Ingestion of Calcium Alginate-containing Food on Postprandial Blood Triglyceride Level and Blood Glucose Level in Adult Males” (pp. 109-114) written by Akiyoshi Sawabe, et al.

Besides, Patent Document 1 discloses an agent for lowering cholesterol in the body containing calcium alginate as an active ingredient, and food and drink and the like containing the active ingredient, and Patent Document 2 discloses a triglyceride-lowering drug for lowering triglyceride in the body containing calcium alginate as an active ingredient, and food and drink and the like containing the active ingredient. Besides, a method for imparting a health function to noodles by using calcium alginate in production of the noodles is also disclosed. For example, Patent Document 3 discloses, as a composition for producing raw rice noodles, a hypoglycemic gluten-free composition for producing raw rice noodles obtained by adding 0.01 to 1% by mass of an alginate such as calcium alginate to a raw rice noodle production raw material in which a rice flour, a silkworm powder, transglutaminase, salt and trehalose are mixed, and Patent Document 4 discloses a method for producing boiled udon (Japanese wheat noodles) that minimally increases a postprandial blood glucose level in which a noodle production raw material containing a wheat flower and an alginate such as sodium alginate is kneaded to prepare an udon dough, and noodle strings obtained therefrom are boiled and then dipped in a calcium solution for performing a calcium alginate gel formation treatment.

On the other hand, regarding production of noodles, various usages of calcium alginate for improving physical properties of produced noodle strings and the like are known. For example, Patent Document 5 discloses a method for producing quick-cooking noodles in which shape retainability is imparted to noodle strings by coating noodles obtained after noodle making with a coating film of calcium alginate, Patent Document 6 discloses a method for producing noodles that do not become soggy but have good texture by dipping a dough containing sodium alginate in a calcium chloride solution in the production of the noodles, and Patent Document 7 discloses a method in which an alginate (sodium salt) is mixed in a food material of noodles or the like to be kneaded, the resultant is soaked in a reaction solution of a metal salt containing calcium such as calcium chloride or calcium lactate to form a coating film by a gelation reaction to be used as a binding agent to impart elasticity.

Besides, Patent Document 8 discloses a method, employed in production of food such as noodles, in which a gelling agent such as sodium alginate is contained in or attached to the food, the resultant is dipped in a liquid containing a metal ion so as to prevent dissolution, swelling and collapse of an inside structure, Patent Document 9 discloses a method, employed in production of noodles made from miscellaneous grains, in which sodium alginate is added to miscellaneous grain flour, followed by mixing, and noodles obtained after noodle making is dipped in a calcium salt aqueous solution to impart a binding property to the resultant noodles, and Patent Document 10 discloses a method, employed in production of heat-cooked noodles, in which sodium alginate is mixed in a noodle production raw material to make noodle strings, and the noodle strings are boiled in hot water containing calcium to form a coating film for preventing cooked noodle strings from binding to one another.

Furthermore, Patent Document 11 discloses a method, employed in production of noodles, for producing noodles feeling good going down in which a noodle improving agent containing calcium alginate is used in a powder raw material for noodle production in a ratio of 0.05 to 2.0% by weight, and preferably 0.1 to 1.0% by weight with respect to the powder raw material to improve hardness, elasticity and chewiness of the noodles without impairing taste of the noodles.

As described above, calcium alginate is conventionally known as a component expected to be added to and used in food and the like as a health function component, and use of this component in noodles as a component for imparting functionally has been disclosed. On the other hand, various uses of calcium alginate itself in production of noodles for improving physical properties of produced noodle strings and the like are also known, and as described above, various use methods employed in production of noodles have been disclosed. Calcium alginate is, however, insoluble in fresh water or a salt solution, and therefore, when calcium alginate is used in production of noodles, there arises a problem that a viscoelastic tissue of noodles is affected, and addition of calcium alginate deteriorates the viscoelasticity of noodle tissue to impair texture. Accordingly, in using the component in production of noodles, the addition of the component to a noodle production raw material deteriorates original physical properties of noodles and texture, and therefore, the use of the component in production of noodles is restricted.

Therefore, as described in the above-described disclosed methods, as a conventional method for using calcium alginate in production of noodles, a method in which an alginate (sodium salt) is mixed in a food material of noodles or the like to be kneaded, and the resultant is soaked in a reaction solution of a metal salt containing calcium such as calcium chloride or calcium lactate to form a coating film on surfaces of noodles or noodle strings by a gelation reaction, or a method in which an amount of calcium alginate added to noodles is limited (Patent Document 3 and Patent Document 11) to avoid deterioration of original physical property of noodles and taste otherwise caused by addition of the component to a noodle production raw material is employed. Accordingly, in the conventional method, even if calcium alginate is added to noodles to impart its health function in production of noodles, the health function of the component cannot be sufficiently exhibited, and alternatively, even when a method for preventing the deterioration of original taste and the like of noodles by a method for forming a coating film by a gelation reaction on surfaces of the noodles or noodle strings is employed, the influence of the coating film of calcium alginate on the original taste and the like of the noodles cannot be avoided under current circumstances.

Under these circumstances, in consideration of needs of provision of health food capable of contributing prevention or treatment of lifestyle-related diseases and the like by imparting health function to food or the like, provision of good-texture healthy boiled noodles excellent in texture by imparting a health function using a health function component such as calcium alginate to noodles, which are originally regarded as healthy food, and in addition, without impairing original texture of boiled noodles is presumed to be regarded as provision of health food meeting the needs of consumers.

### PRIOR ART DOCUMENTS

### Patent Documents

Patent Document 1: Japanese unexamined Patent Application Publication No. 2016-3194

Patent Document 2: Japanese unexamined Patent Application Publication No. 2017-95403

Patent Document 3: Japanese unexamined Patent Application Publication No. 2012-125245

Patent Document 4: Japanese unexamined Patent Application Publication No. 2014-54

Patent Document 5: Japanese unexamined Patent Application Publication No. 60-012946

Patent Document 6: Japanese unexamined Patent Application Publication No. 62-79749

Patent Document 7: Japanese unexamined Patent Application Publication No. 62-296849

Patent Document 8: Japanese unexamined Patent Application Publication No. 63-192353

Patent Document 9: Japanese unexamined Patent Application Publication No. 06-233660

Patent Document 10: Japanese unexamined Patent Application Publication No. 2002-281923

Patent Document 11: Japanese unexamined Patent Application Publication No. 2004-147576

### Non-Patent Documents

Non-patent Document 1: “Shokuseikatsu Kenkyu (Dietary Scientific Research in Japanese)”, issued in 2013, vol. 33, No. 2, pp. 102-108, 109-114

### SUMMARY OF THE INVENTION

### Object to be Solved by the Invention

An object of the present invention is to provide good-texture healthy boiled noodles having an effective health function of calcium alginate and excellent in texture by imparting a health function of calcium alginate to noodles, and in addition, without impairing original texture of boiled noodles, and furthermore, to provide good-texture healthy raw noodles for boiled cooking excellent in texture by imparting the health function of calcium alginate to noodles, and in addition, without impairing original texture of noodles.

### Means to Solve the Object

In order to solve the above-described object, while the present inventors are making earnest studies on a method for providing good-texture healthy boiled noodles having an effective health function of calcium alginate and excellent in texture by using a health function of calcium alginate, by mixing calcium alginate insoluble in water to a noodle production raw material to impart the health function of calcium alginate, and in addition without impairing original texture of boiled noodles, it has been found that boiled noodles retaining a good texture of noodles can be produced, with the health function of calcium alginate effectively imparted to the noodles, and in addition, without deteriorating original texture of noodles, by employing, in a method for producing boiled noodles comprising a mixing and kneading step of kneading a dough by adding kneading water to a powder raw material for noodle production containing wheat flour, a noodle making step of forming the dough into noodle strings, and a boiling step of boiling the noodle strings, a method in which a calcium alginate powder having a fine grain size designated as a 270-mesh pass pulverized calcium alginate powder is mixed in the powder raw material for noodle production in a specific ratio of 4 to 8 parts by mass based on 100 parts by mass of the powder raw material for noodle production, a dough is kneaded by adding kneading water to the powder raw material for noodle production, and the resultant is subjected to noodle making and boiling, and thus, the present invention was accomplished. Furthermore, in the present invention, it has been found that when the method in which a calcium alginate powder having a fine grain size designated as a 270-mesh pass pulverized calcium alginate powder is mixed in a powder raw material for noodle production in a specific ratio of 4 to 8 parts by mass based on 100 parts by mass of the powder raw material for noodle production, a dough is kneaded by adding kneading water to the powder raw material for noodle production, and then subjected to noodle making is employed, good-texture healthy raw noodles for boiled cooking excellent in texture can be provided with the health function of calcium alginate imparted, and in addition, without impairing the original texture of noodles, and thus, the present invention was accomplished.

Specifically, the present invention provides a method for producing healthy boiled noodles having a health function of calcium alginate and retaining a good texture of noodles, in which in a method for producing boiled noodles comprising (A) a mixing and kneading step of kneading a dough by adding kneading water to a powder raw material for noodle production obtained through a mixture of raw materials containing wheat flour; (B) a noodle making step of forming the dough into noodle strings; and (C) a boiling step of boiling the noodle strings and cooling the resultant to prepare boiled noodles, a 270-mesh pass pulverized calcium alginate powder is mixed in the powder raw material for noodle production in a ratio of 4 to 8 parts by mass based on 100 parts by mass of the powder raw material for noodle production. Besides, the present invention provides a method for producing healthy raw noodles with a health function of calcium alginate and retaining a good texture of noodles, in which in a method for producing raw noodles for boiled cooking comprising (A) a mixing and kneading step of kneading a dough by adding kneading water to a powder raw material for noodle production obtained through a mixture of raw materials containing wheat flour; (B) a noodle making step of forming the dough into noodle strings; and (C) a step of cutting the noodle strings into a predetermined amount, a 270-mesh pass pulverized calcium alginate powder is mixed, for production, in the powder raw material for noodle production in a ratio of 4 to 8 parts by mass based on 100 parts by mass of the powder raw material for noodle production.

The healthy boiled noodles produced by the method of the present invention effectively retains a health function of calcium alginate, such as an effect of suppressing postprandial blood glucose level increase, and in addition, deterioration of viscoelasticity and the like of noodle tissue and deterioration of texture otherwise caused in adding calcium alginate in the production of noodles are not caused, and thus, good-texture healthy boiled noodles retaining original texture of noodles are provided.

Noodles such as udon are served in the form of boiled noodles, and in one aspect of the boiled noodles, soft texture felt in eating is appreciated, and they are easily digested because of the softness, and hence easily increase blood glucose concentration. The method of the present invention is applied to production of boiled noodles of udon and the like, and the boiled noodles produced by the method provide boiled noodles to which the health function of calcium alginate is imparted without causing deterioration of the viscoelasticity of noodle tissue and the like and deterioration of texture in the production of the noodles with the original good texture of noodles retained, and as described above, the problem of the boiled noodles themselves of the increase of blood glucose concentration is solved, and thus good-texture healthy boiled noodles with which the increase of blood glucose concentration is suppressed are provided.

Specifically, the present invention encompasses the following methods:

[1] A method for producing healthy boiled noodles having a health function of calcium alginate and retaining a good texture of noodles, wherein in a method for producing boiled noodles comprising (A) a mixing and kneading step of kneading a dough by adding kneading water to a powder raw material for noodle production obtained through a mixture of raw materials containing wheat flour; (B) a noodle making step of forming the dough into noodle strings; and (C) a boiling step of boiling and cooling the noodle strings to prepare boiled noodles, a 270-mesh pass pulverized calcium alginate powder is mixed in the powder raw material for noodle production in a ratio of 4 to 8 parts by mass based on 100 parts by mass of the powder raw material for noodle production.

[2] The method for producing healthy boiled noodles according to [1] described above, wherein the health function of the healthy boiled noodles is an effect of suppressing postprandial blood glucose level increase.

[3] The method for producing healthy boiled noodles according to [1] or [2] described above, wherein the boiled noodles are selected from boiled udon, chilled boiled buckwheat noodles, chilled boiled Chinese noodles and frozen boiled pasta.

[4] A method for producing healthy raw noodles having a health function of calcium alginate and retaining a good texture of noodles, wherein in a method for producing raw noodles for boiled cooking comprising (A) a mixing and kneading step of kneading a dough by adding kneading water to a powder raw material for noodle production obtained through a mixture of raw materials containing wheat flour; (B) a noodle making step of forming the dough into noodle strings; and (C) a step of cutting the noodle strings into a predetermined amount, a 270-mesh pass pulverized calcium alginate powder is mixed, for production, in the powder raw material for noodle production in a ratio of 4 to 8 parts by mass based on 100 parts by mass of the powder raw material for noodle production. [5] The method for producing raw noodles of healthy raw noodles according to [4] described above, wherein the raw noodles are selected from chilled raw udon, chilled raw buckwheat noodles, chilled raw pasta and chilled raw Chinese noodles.

[6] A method for allowing boiled noodles or raw noodles to retain a good texture of noodles and for imparting a health function of calcium alginate thereto, wherein in a method for producing boiled noodles comprising (A) a mixing and kneading step of kneading a dough by adding kneading water to a powder raw material for noodle production obtained through a mixture of raw materials containing wheat flour; (B) a noodle making step of forming the dough into noodle strings; and (C) a boiling step of boiling and cooling the noodle strings to prepare boiled noodles, or in a method for producing raw noodles for boiled cooking comprising (A) a mixing and kneading step of kneading a dough by adding kneading water to a powder raw material for noodle production obtained through a mixture of raw materials containing wheat flour; (B) a noodle making step of forming the dough into noodle strings; and (C) a step of cutting the noodle strings into a predetermined amount, a 270-mesh pass pulverized calcium alginate powder is mixed in the powder raw material for noodle production in a ratio of 4 to 8 parts by mass based on 100 parts by mass of the powder raw material for noodle production.

### Effect of the Invention

The present invention provides good-texture healthy boiled noodles retaining original texture of noodles having a health function of calcium alginate such as an effect of suppressing postprandial blood glucose level increase effectively retained, and in addition, without causing, in production of the noodles, deterioration of viscoelasticity and the like of noodle tissue and deterioration of texture otherwise caused in adding calcium alginate. When the method of the present invention is applied to production of boiled udon, boiled udon in which calcium alginate does not elute during a boiling step so that viscosity increase of hot water used for boiling can be restrained, in which deterioration of texture corresponding to a characteristic of udon, such as softness and chewy texture, can be prevented, and with which rapid increase of postprandial blood glucose level can be suppressed are provided.

### BRIEF DESCRIPTION OF DRAWINGS

FIG. 1 is a diagram illustrating comparison in transition of a change in blood glucose level between that obtained after ingestion of udon in which a calcium alginate powder is kneaded and that obtained after ingestion of udon in which a calcium alginate powder is not kneaded.

FIG. 2 is a diagram illustrating comparison in transition of a change in blood glucose level between that obtained after ingestion of udon in which a calcium alginate powder is kneaded and that obtained after ingestion of udon in which a calcium alginate powder is not kneaded and a calcium alginate powder.

FIG. 3 is a diagram illustrating comparison in transition of a change in blood glucose level among that obtained after ingestion of udon in which a calcium alginate powder is kneaded, that obtained after ingestion of udon in which sodium alginate is kneaded, and that obtained after ingestion of udon having been subjected to a calcium alginate gel formation treatment.

FIG. 4 is a diagram illustrating comparison in transition of a change in blood glucose level among those obtained after ingestion of udon in which calcium alginate powders having different grain sizes are respectively kneaded.

FIG. 5 is a diagram illustrating comparison in transition of a change in blood glucose level obtained in Example 5.

FIG. 6 is a diagram illustrating blood glucose levels obtained at elapsed times after ingestion in Example 6.

FIG. 7 is a diagram illustrating each Ca alginate addition percentage and an evaluation score (hardness) obtained in Example 7.

FIG. 8 is a diagram illustrating each Ca alginate addition percentage and an evaluation score (chewy texture) obtained in Example 7.

FIG. 9 is a diagram illustrating each Ca alginate addition percentage and an evaluation score (powderiness) obtained in Example 7.

FIG. 10 is a diagram illustrating each Ca alginate addition percentage and an evaluation score (comprehensive texture) obtained in Example 7.

FIG. 11 is a diagram illustrating blood glucose levels obtained at elapsed times after ingestion in Example 8.

FIG. 12 is a diagram illustrating blood glucose levels obtained at elapsed times after ingestion in Example 9.

FIG. 13 is a diagram illustrating blood glucose levels obtained at elapsed times after ingestion in Example 10.

FIG. 14 is a diagram illustrating blood glucose levels obtained at elapsed times after ingestion in Example 11.

FIG. 15 is a diagram illustrating blood glucose levels obtained at elapsed times after ingestion in Example 12.

FIG. 16 is a diagram illustrating blood glucose levels obtained at elapsed times after ingestion in Example 13.

FIG. 17 is a diagram illustrating blood glucose levels obtained at elapsed times after ingestion in Example 14.

### MODE OF CARRYING OUT THE INVENTION

The present invention provides a method for producing healthy boiled noodles having a health function of calcium alginate and retaining a good texture of noodles, in which in a method for producing boiled noodles comprising (A) a mixing and kneading step of kneading a dough by adding kneading water to a powder raw material for noodle production obtained through a mixture of raw materials containing wheat flour; (B) a noodle making step of forming the dough into noodle strings; and (C) a boiling step of boiling the noodle strings and cooling the resultant to prepare boiled noodles, a 270-mesh pass pulverized calcium alginate powder is mixed in the powder raw material for noodle production in a ratio of 4 to 8 parts by mass based on 100 parts by mass of the powder raw material for noodle production, and a method for producing healthy raw noodles with a health function of calcium alginate and retaining a good texture of noodles, in which in a method for producing raw noodles for boiled cooking comprising (A) a mixing and kneading step of kneading a dough by adding kneading water to a powder raw material for noodle production obtained through a mixture of raw materials containing wheat flour; (B) a noodle making step of forming the dough into noodle strings; and (C) a step of cutting the noodle strings into a predetermined amount, a 270-mesh pass pulverized calcium alginate powder is mixed, for production, in the powder raw material for noodle production in a ratio of 4 to 8 parts by mass based on 100 parts by mass of the powder raw material for noodle production.

In production of boiled noodles of the present invention, production raw materials, production procedures, production conditions and the like of the boiled noodles, or production raw materials, production procedures, production conditions and the like of raw noodles are not different from those employed in a known method for producing boiled noodles or raw noodles except that the 270-mesh pass pulverized calcium alginate powder is mixed in the powder raw material for noodle production in a ratio of 4 to 8 parts by mass based on 100 parts by mass of the powder raw material for noodle production. In the noodle production raw material, not only a wheat flour but also an auxiliary raw material, a mixed raw material and an additive used in known production of noodles can be appropriately mixed or added in a range not changing the effect of the present invention.

In the production of boiled noodles or raw noodles of the present invention, as a noodle production wheat flour raw material, a known wheat flour raw material used in production of noodles can be used, and for example, a wheat flour used as a raw material for producing udon is not limited to a specific wheat flour in principle, and specific examples of a wheat flour particularly suitable for the method of the present invention include wheat flours obtained from imported wheat of ASW (Australian standard white), wheat from Hokkaido such as Kitahonami, low amylose wheat such as Chikugoizumi, wheat from Kyushu, and other domestic wheats, and a particularly preferable wheat flour is a wheat flour obtained by blending low amylose wheat with ASW or Kitahonami. In order to take advantage of flavor of the wheat flour, the wheat flour can be mixed in a mixing amount of 50% by mass or more, and particularly preferably 70% by mass of more of the powder raw material of boiled udon.

In the present invention, starch having a higher gelatinization viscosity than the wheat flour used as the raw material can be mixed as a part of the powder raw material to increase chewy texture and viscoelasticity of the texture. For example, oxidized starch such as tapioca starch or waxy corn starch, or modified starch having been subjected to a treatment for increasing the gelatinization viscosity through acetylation, etherification or the like regardless of an origin raw material can be used. A mixing amount of such starch is not especially limited, and is preferably in a range of 5 to 30% by mass, and more preferably 10 to 20% by mass with respect to a total amount of the wheat flour and the starch.

Besides, in the present invention, starch more indigestible than the wheat flour used as the raw material can be mixed as a part of the powder raw material. For example, high amylose corn starch, or modified starch minimally digested and absorbed by phosphate acid crosslinking or the like regardless of an origin raw material can be used. A mixing amount of such starch is not especially limited, and is preferably in a range of 10 to 50% by mass with respect to the powder raw material.

When a mixing ratio of starch is increased, vital gluten can be added as a part of the powder raw material for purposes of compensate relatively insufficient gluten. Besides, ones for improving noodle making properties such as fats and oils, and an emulsion can be used.

In the method for producing boiled noodles of the present invention, the 270-mesh pass pulverized calcium alginate powder is mixed in the powder raw material for noodle production in a ratio of 4 to 8 parts by mass based on 100 parts by mass of the powder raw material for noodle production. As an origin raw material of the powdered calcium alginate, any species can be used as long as it is a brown alga, but nigrescence belonging to the class Phaeophycease, the order Laminariales, the family Lessoniaceae is preferred, and although its molecular weight is not limited to a specific molecular weight range, the molecular weight is said to be about 700000 KDa. As the grain size of the powdered calcium alginate, a 270-mesh pass pulverized powder is used, and as the grain size of the powdered calcium alginate, a pulverized one having a value, measured using LA-920, a laser diffraction/scattering grain size distribution analyzer manufactured by Horiba Ltd., in a range of 1 to 50 μm is preferred, and a range of 1 to 30 μm is further preferred. Preparation of the pulverized powder can be performed by pulverizing a powder of calcium alginate by an ordinary method using grinding means such as a ball mill, and classifying the pulverized powder by classifying means such as a mesh screen into a predetermined mesh pass pulverized powder. Alternatively, it is appropriately available from commercially available standardized products.

Regarding the mixing ratio of mixing the powdered calcium alginate, it is mixed in a ratio of 4 to 8 parts by mass based on 100 parts by mass of the powder raw material for noodle production, and it is mixed more preferably in a ratio of 5 to 6 parts by mass. When the mixing amount of the calcium alginate powder is less than 4 parts by mass, it is apprehended that the effect of suppressing postprandial blood glucose level increase may be insufficient, and when it exceeds 8 parts by mass, the texture may be deteriorated in some cases.

In the mixing and kneading step of kneading a dough by adding kneading water to the powder raw material for noodle production in the method for producing boiled noodles or raw noodles of the present invention, not only a salt but also potassium chloride can be used in the kneading water added to the powder raw material. Besides, when a salt is singly used, its concentration is in a range of preferably 2 to 12% by mass, and more preferably 8 to 10% by mass.

In the kneading step in the method for producing boiled noodles or raw noodles of the present invention, after premixing the powder raw material with the powdered calcium alginate, the kneading water can be added to knead the resultant with a vacuum mixer. In order to effectively perform the kneading step, for example, a method in which a ribbon mixer is provided on a front stage for highly homogenizing the powdered calcium alginate can be employed.

The noodle making step in the method for producing boiled noodles or raw noodles of the present invention can be performed using, for example, a roll type noodle belt forming machine or a roll mill, or can be freely performed using a hand-made style noodle making machine or an extrusion type noodle belt forming machine. Incidentally, a noodle belt stiffened by noodle making can be finely cut into noodle strings through a rolling mill and a noodle slitter after appropriate standing time. In the production process for raw noodles of the present invention, after the noodle making step (B) of forming the dough into noodle strings and the step (C) of cutting the noodle strings into a predetermined amount, a powder sprinkling step (D) of sprinkling starch over the noodle strings cut into a predetermined amount for preventing adhesion among the noodle strings can be provided.

In the boiling step in the method for producing boiled noodles of the present invention, noodle strings divided into the same number may be cut into a predetermined length corresponding to each serving unit to be boiled and cooled, or the noodle strings are boiled and cooled in the lump, and then weighed. A temperature of water used for boiling may be adjusted to 98° C. or more, and pH may be adjusted to 5.0 to 6.0. Boiled udon may be subjected to a dipping treatment in a mixture of cooling water with an organic acid for increasing a preserving property at the time of chilled distribution, or may be subjected to a secondary heat treatment using steam, hot water, microwaves or the like after sealed packaging for further increasing the preserving property. Besides, in a case of frozen noodles, quick freezing is preferred for retaining texture obtained immediately after boiling, and freezing may be completed within 30 minutes after boiling.

In the present invention, pulverized calcium alginate is contained within udon in a homogeneously dispersed state, and thus, an operational advantage of maximizing the function of calcium alginate without missing the timing of digesting and absorbing udon can be obtained. Besides, pulverized calcium alginate does not excessively absorb water like water-soluble alginate, and hence does not inhibit gluten film formation in a dough in the kneading step, and therefore, an effect that deterioration in slippiness and good feeling on the tongue can be prevented with retaining appropriate hardness and elasticity in the texture of udon can be obtained.

### EXAMPLES

Now, Examples 1 to 5 will be given to more specifically describe the present invention, but the technical scope of the present invention is not limited to the exemplified description.

### Example 1

The effect of suppressing postprandial blood glucose level increase of udon in which 5 parts by weight (with respect to a powder raw material) or 8 parts by weight (with respect to a powder raw material) of 270-mesh pass Ca alginate is added is examined.

[Preparation of Udon Samples]

After powder raw materials shown in [Table 1] were respectively homogeneously mixed, kneading water shown in [Table 1] was added respectively thereto, and the resultants were kneaded at a decompression degree of 80 kPa for 10 to 16 minutes to obtain kneaded doughs in a crumbled state at a temperature of 28 to 34° C. Each of the kneaded doughs was formed into crude noodle belts each having a thickness of 10 mm using a noodle belt forming roll to be compounded with the thickness kept, the resultant was allowed to stand at 25° C. for 60 minutes under a condition where it was not dried, was rolled into a thickness of 3.6 mm using a three-stage flat roll, and then finely cut into noodle strings using cutting teeth with a grove width of 3.5 mm.

The thus obtained noodle strings were boiled in hot water used for boiling at 98° C. for 13 minutes, then somewhat cooled by putting them in water at 15° C. twice, and dipped in a 0.4% by mass acetic acid solution at 5° C. for 1 minute and 45 seconds. Thereafter, the solution remaining thereon was rapidly removed, 180 g thereof corresponding to each serving was sealed with a polyethylene film, subjected to steam sterilization at 85° C. for 30 minutes, and rapidly cooled in a refrigerator at 10° C. to prepare chilled boiled udon having a boiled noodle moisture shown in [Table 1].

TABLE 1

| | Sample Type | | | | | |
|---|---|---|---|---|---|---|
| | | | Operation | | Operation | |
| Item | Control | | Plot 1 | | Plot 2 | |
| (Powder Raw Materials) | | | | | | |
| Wheat Flour mainly | 2520 | g | 0 | g | 0 | g |
| composed of ASW | | | | | | |
| Wheat Flour mainly | 0 | g | 2760 | g | 2760 | g |
| composed of Kitahonami | | | | | | |
| Acetylated Tapioca Starch | 480 | g | 240 | g | 240 | g |
| Calcium Alginate | 0 | g | 150 | g | 240 | g |
| Total of Powders | 3000 | g | 3150 | g | 3240 | g |
| (Kneading Water) | | | | | | |
| 10% Salt Solution | 1298 | g | 1474 | g | 1570 | g |
| Boiling Time | 13 | min | 13 | min | 13 | min |

| | | | |
|---|---|---|---|
| Boiled Noodle Moisture | 67.4% | 67.4% | 67.2% |

[Measurement Method for Blood Glucose Level]

Three boiled udon samples in total of a control, an operation plot 1 and an operation plot 2 were prepared, and the measurement of postprandial blood glucose level was entrusted to General Incorporated Association Kendai Translational Research Center. Subjects were 15 healthy males and females (healthy volunteers) over 20 years old including a person having a fasting blood glucose level corresponding to borderline diabetes. For cooking, 180 g corresponding to each serving of each sample was boiled for 3 minutes in boiling water to be served as kake udon (udon in a hot soup) with no ingredients. Conditions were adjusted so that the udon was able to be finished in 5 minutes after serving, and the blood glucose level was measured 15 minutes, 30 minutes, 45 minutes, 60 minutes, 90 minutes and 120 minutes after finishing the ingestion.

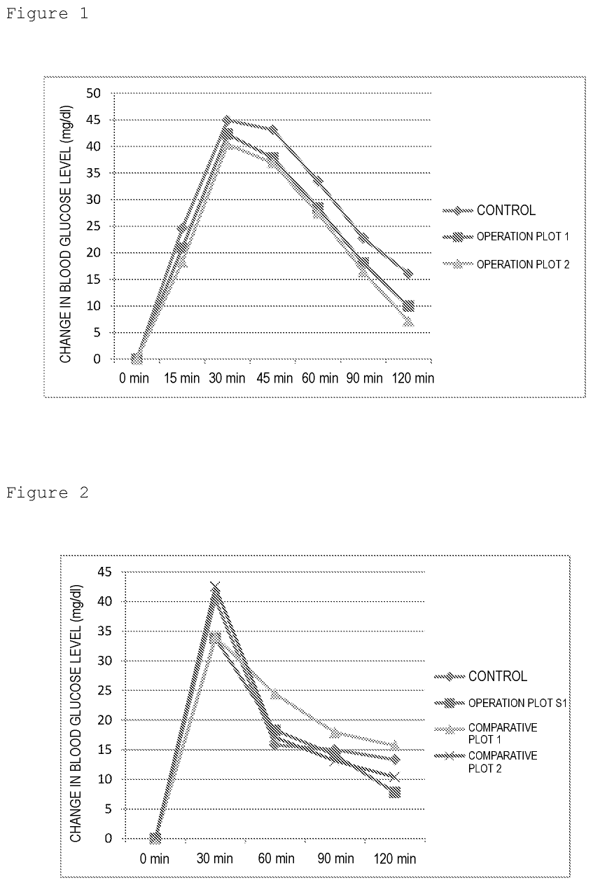

The thus obtained blood glucose level is shown in [Table 2], a change in the blood glucose level is shown in [Table 3], a difference (ΔCₘₐₓ) between a maximum blood glucose level and preprandial blood glucose level is shown in [Table 4], and a product (ΔAUC) of the blood glucose level and the time calculated using the preprandial blood glucose level as a baseline is shown in [Table 5]. Besides, transition of an average of the blood glucose level is illustrated in a graph of FIG. 1.

TABLE 2

| | | Control | | | | | | | Operation Plot 1 | | | | | | |
|---|---|---|---|---|---|---|---|---|---|---|---|---|---|---|---|
| | | Time Elapsed after Ingestion | | | | | | | | | | | | | |
| | | | 15 | 30 | 45 | 60 | 90 | 120 | | 15 | 30 | 45 | 60 | 90 | 120 |
| | | Prior | min | min | min | min | min | min | Prior | min | min | min | min | min | min |
| Subject | 1 | 95.5 | 110.0 | 127.5 | 117.0 | 113.0 | 94.5 | 107.0 | 94.5 | 104.0 | 125.0 | 128.0 | 107.0 | 107.0 | 103.0 |
| No. | 2 | 90.5 | 100.5 | 129.5 | 113.5 | 92.5 | 106.5 | 104.0 | 87.0 | 101.5 | 125.0 | 105.0 | 93.0 | 102.0 | 101.0 |
| | 3 | 80.0 | 127.5 | 148.5 | 142.5 | 124.5 | 120.5 | 112.0 | 90.0 | 117.5 | 132.0 | 135.5 | 146.0 | 108.5 | 103.0 |
| | 4 | 84.0 | 99.0 | 110.0 | 128.0 | 137.0 | 123.0 | 108.0 | 81.5 | 100.0 | 136.5 | 139.5 | 142.5 | 113.5 | 97.5 |
| | 5 | 84.5 | 99.5 | 120.5 | 140.0 | 142.5 | 111.5 | 95.5 | 83.5 | 105.5 | 128.5 | 115.5 | 92.5 | 98.0 | 103.0 |
| | 6 | 79.5 | 114.5 | 142.0 | 144.0 | 127.5 | 108.5 | 99.0 | 81.0 | 110.5 | 131.5 | 129.0 | 113.5 | 107.5 | 81.5 |
| | 7 | 90.0 | 101.0 | 132.0 | 139.5 | 126.5 | 107.5 | 110.5 | 93.5 | 107.5 | 137.0 | 137.5 | 141.0 | 124.5 | 112.5 |
| | 8 | 99.5 | 105.5 | 128.0 | 131.5 | 108.0 | 110.0 | 105.5 | 104.0 | 123.5 | 131.0 | 130.0 | 115.0 | 106.0 | 107.5 |
| | 9 | 95.0 | 133.0 | 147.0 | 147.0 | 139.5 | 127.5 | 106.5 | 105.5 | 123.5 | 152.5 | 151.0 | 120.0 | 112.5 | 99.5 |
| | 10 | 86.0 | 126.5 | 150.5 | 168.5 | 135.0 | 120.5 | 104.0 | 88.5 | 123.5 | 147.5 | 139.5 | 131.0 | 117.5 | 114.0 |
| | 11 | 89.5 | 126.5 | 141.0 | 110.0 | 106.0 | 97.5 | 96.0 | 90.0 | 110.5 | 131.0 | 122.5 | 110.5 | 104.0 | 93.5 |
| | 12 | 77.5 | 106.0 | 107.5 | 87.5 | 97.0 | 99.5 | 99.0 | 91.5 | 111.0 | 126.5 | 115.0 | 105.0 | 93.0 | 88.0 |
| | 13 | 93.5 | 126.0 | 140.0 | 129.5 | 118.0 | 103.5 | 98.5 | 98.0 | 120.5 | 129.5 | 125.0 | 139.0 | 116.5 | 117.0 |
| | 14 | 92.0 | 106.0 | 135.0 | 136.5 | 118.0 | 107.5 | 111.0 | 90.0 | 110.0 | 139.5 | 128.5 | 109.5 | 105.0 | 93.5 |
| | 15 | 85.5 | 109.5 | 137.5 | 134.5 | 139.0 | 125.5 | 107.0 | 95.5 | 118.0 | 136.0 | 140.5 | 135.0 | 129.5 | 109.0 |

| | | | | | | | | | | | | | | |
|---|---|---|---|---|---|---|---|---|---|---|---|---|---|---|
| Average of | 88.2 | 112.7 | 133.1 | 131.3 | 121.6 | 110.9 | 104.2 | 91.6 | 112.5 | 133.9 | 129.5 | 120.0 | 109.7 | 101.6 |
| Subjects | | | | | | | | | | | | | | |

| | | | | | | |
|---|---|---|---|---|---|---|
| T-test against Control Food at Corresponding | 91% | 77% | 64% | 72% | 64% | 32% |
| Time (Probability according to t-distribution) | | | | | | |

unit: mg/dl

| | | Control | | | | | | | Operation Plot 2 | | | | | | |
|---|---|---|---|---|---|---|---|---|---|---|---|---|---|---|---|
| | | Time Elapsed after Ingestion | | | | | | | | | | | | | |
| | | | 15 | 30 | 45 | 60 | 90 | 120 | | 15 | 30 | 45 | 60 | 90 | 120 |
| | | Prior | min | min | min | min | min | min | Prior | min | min | min | min | min | min |
| Subject | 1 | 95.5 | 110.0 | 127.5 | 117.0 | 113.0 | 94.5 | 107.0 | 97.5 | 109.5 | 118.5 | 121.5 | 118.0 | 117.0 | 109.0 |
| No. | 2 | 90.5 | 100.5 | 129.5 | 113.5 | 92.5 | 106.5 | 104.0 | 80.0 | 89.5 | 112.0 | 111.5 | 98.5 | 94.0 | 91.0 |
| | 3 | 80.0 | 127.5 | 148.5 | 142.5 | 124.5 | 120.5 | 112.0 | 100.0 | 114.5 | 132.5 | 126.5 | 125.0 | 111.0 | 98.5 |
| | 4 | 84.0 | 99.0 | 110.0 | 128.0 | 137.0 | 123.0 | 108.0 | 87.5 | 108.0 | 154.0 | 163.0 | 150.0 | 126.0 | 97.5 |
| | 5 | 84.5 | 99.5 | 120.5 | 140.0 | 142.5 | 111.5 | 95.5 | 92.0 | 100.0 | 129.5 | 138.0 | 137.5 | 105.5 | 96.0 |
| | 6 | 79.5 | 114.5 | 142.0 | 144.0 | 127.5 | 108.5 | 99.0 | 70.0 | 104.0 | 123.0 | 113.5 | 108.0 | 81.5 | 82.0 |
| | 7 | 90.0 | 101.0 | 132.0 | 139.5 | 126.5 | 107.5 | 110.5 | 92.5 | 106.0 | 124.0 | 124.5 | 104.0 | 102.0 | 93.0 |
| | 8 | 99.5 | 105.5 | 128.0 | 131.5 | 108.0 | 110.0 | 105.5 | 101.5 | 118.0 | 124.5 | 117.0 | 106.0 | 113.0 | 106.0 |
| | 9 | 95.0 | 133.0 | 147.0 | 147.0 | 139.5 | 127.5 | 106.5 | 96.5 | 113.5 | 147.5 | 132.5 | 116.5 | 127.5 | 98.0 |
| | 10 | 86.0 | 126.5 | 150.5 | 168.5 | 135.0 | 120.5 | 104.0 | 95.5 | 122.0 | 150.0 | 137.5 | 117.0 | 121.0 | 108.0 |
| | 11 | 89.5 | 126.5 | 141.0 | 110.0 | 106.0 | 97.5 | 96.0 | 91.5 | 116.0 | 131.5 | 123.0 | 121.5 | 93.5 | 103.5 |
| | 12 | 77.5 | 106.0 | 107.5 | 87.5 | 97.0 | 99.5 | 99.0 | 96.0 | 114.5 | 132.0 | 119.5 | 102.5 | 89.0 | 85.0 |
| | 13 | 93.5 | 126.0 | 140.0 | 129.5 | 118.0 | 103.5 | 98.5 | 96.5 | 116.5 | 140.5 | 126.0 | 114.5 | 104.0 | 96.0 |
| | 14 | 92.0 | 106.0 | 135.0 | 136.5 | 118.0 | 107.5 | 111.0 | 84.5 | 110.5 | 129.5 | 137.0 | 123.0 | 118.5 | 113.0 |
| | 15 | 85.5 | 109.5 | 137.5 | 134.5 | 139.0 | 125.5 | 107.0 | 89.5 | 102.0 | 128.0 | 133.5 | 140.0 | 114.0 | 101.5 |

| | | | | | | | | | | | | | | |
|---|---|---|---|---|---|---|---|---|---|---|---|---|---|---|
| Average of | 88.2 | 112.7 | 133.1 | 131.3 | 121.6 | 110.9 | 104.2 | 91.4 | 109.6 | 131.8 | 128.3 | 118.8 | 107.8 | 98.5 |
| Subjects | | | | | | | | | | | | | | |

| | | | | | | |
|---|---|---|---|---|---|---|
| T-test against Control Food at Corresponding | 23% | 77% | 55% | 40% | 31% | 2% |
| Time (Probability according to t-distribution) | | | | | | |

TABLE 3
unit: mg/dl

| | | Control | | | | | | Operation Plot 1 | | | | | | Operation Plot 2 | | | | | |
|---|---|---|---|---|---|---|---|---|---|---|---|---|---|---|---|---|---|---|---|
| | | Time Elapsed after Ingestion | | | | | | | | | | | | | | | | | |
| | | 15 | 30 | 45 | 60 | 90 | 120 | 15 | 30 | 45 | 60 | 90 | 120 | 15 | 30 | 45 | 60 | 90 | 120 |
| | | min | min | min | min | min | min | min | min | min | min | min | min | min | min | min | min | min | min |
| Subject | 1 | 14.5 | 32.0 | 21.5 | 17.5 | −1.0 | 11.5 | 9.5 | 30.5 | 33.5 | 12.5 | 12.5 | 8.5 | 12.0 | 21.0 | 24.0 | 20.5 | 19.5 | 11.5 |
| No. | 2 | 10.0 | 39.0 | 23.0 | 2.0 | 16.0 | 13.5 | 14.5 | 38.0 | 18.0 | 6.0 | 15.0 | 14.0 | 9.5 | 32.0 | 31.5 | 18.5 | 14.0 | 11.0 |
| | 3 | 47.5 | 68.5 | 62.5 | 44.5 | 40.5 | 32.0 | 27.5 | 42.0 | 45.5 | 56.0 | 18.5 | 13.0 | 14.5 | 32.5 | 26.5 | 25.0 | 11.0 | −1.5 |
| | 4 | 15.0 | 26.0 | 44.0 | 53.0 | 39.0 | 24.0 | 18.5 | 55.0 | 58.0 | 61.0 | 32.0 | 16.0 | 20.5 | 66.5 | 75.5 | 62.5 | 38.5 | 10.0 |
| | 5 | 15.0 | 36.0 | 55.5 | 58.0 | 27.0 | 11.0 | 22.0 | 45.0 | 32.0 | 9.0 | 14.5 | 19.5 | 8.0 | 37.5 | 46.0 | 45.5 | 13.5 | 4.0 |
| | 6 | 35.0 | 62.5 | 64.5 | 48.0 | 29.0 | 19.5 | 29.5 | 50.5 | 48.0 | 32.5 | 26.5 | 0.5 | 34.0 | 53.0 | 43.5 | 38.0 | 11.5 | 12.0 |
| | 7 | 11.0 | 42.0 | 49.5 | 36.5 | 17.5 | 20.5 | 14.0 | 43.5 | 44.0 | 47.5 | 31.0 | 19.0 | 13.5 | 31.5 | 32.0 | 11.5 | 9.5 | 0.5 |
| | 8 | 6.0 | 28.5 | 32.0 | 8.5 | 10.5 | 6.0 | 19.5 | 27.0 | 26.0 | 11.0 | 2.0 | 3.5 | 16.5 | 23.0 | 15.5 | 4.5 | 11.5 | 4.5 |
| | 9 | 38.0 | 52.0 | 52.0 | 44.5 | 32.5 | 11.5 | 18.0 | 47.0 | 45.5 | 14.5 | 7.0 | −6.0 | 17.0 | 51.0 | 36.0 | 20.0 | 31.0 | 1.5 |
| | 10 | 40.5 | 64.5 | 82.5 | 49.0 | 34.5 | 18.0 | 35.0 | 59.0 | 51.0 | 42.5 | 29.0 | 25.5 | 26.5 | 54.5 | 42.0 | 21.5 | 25.5 | 12.5 |
| | 11 | 37.0 | 51.5 | 20.5 | 16.5 | 8.0 | 6.5 | 20.5 | 41.0 | 32.5 | 20.5 | 14.0 | 3.5 | 24.5 | 40.0 | 31.5 | 30.0 | 2.0 | 12.0 |
| | 12 | 28.5 | 30.0 | 10.0 | 19.5 | 22.0 | 21.5 | 19.5 | 35.0 | 23.5 | 13.5 | 1.5 | −3.5 | 18.5 | 36.0 | 23.5 | 6.5 | −7.0 | −11.0 |
| | 13 | 32.5 | 46.5 | 36.0 | 24.5 | 10.0 | 5.0 | 22.5 | 31.5 | 27.0 | 41.0 | 18.5 | 19.0 | 20.0 | 44.0 | 29.5 | 18.0 | 7.5 | −0.5 |
| | 14 | 14.0 | 43.0 | 44.5 | 26.0 | 15.5 | 19.0 | 20.0 | 49.5 | 38.5 | 19.5 | 15.0 | 3.5 | 26.0 | 45.0 | 52.5 | 38.5 | 34.0 | 28.5 |
| | 15 | 24.0 | 52.0 | 49.0 | 53.5 | 40.0 | 21.5 | 22.5 | 40.5 | 45.0 | 39.5 | 34.0 | 13.5 | 12.5 | 38.5 | 44.0 | 50.5 | 24.5 | 12.0 |

| | | | | | | | | | | | | | | | | | | |
|---|---|---|---|---|---|---|---|---|---|---|---|---|---|---|---|---|---|---|
| Average of | 24.6 | 44.9 | 43.1 | 33.4 | 22.7 | 16.1 | 20.9 | 42.3 | 37.9 | 28.4 | 18.1 | 10.0 | 18.2 | 40.4 | 36.9 | 27.4 | 16.4 | 7.1 |
| Subjects | | | | | | | | | | | | | | | | | | |

| | | | | | | | | | | | | |
|---|---|---|---|---|---|---|---|---|---|---|---|---|
| T-test against Control Food at Corresponding | 18% | 44% | 16% | 28% | 16% | 6% | 6% | 29% | 23% | 13% | 11% | 1% |
| Time (Probability according to t-distribution) | | | | | | | | | | | | |

TABLE 4
unit: mg/dl

| | | | Operation | Operation |
|---|---|---|---|---|
| ΔCₘₐₓ | | Control | Plot 1 | Plot 2 |
| Subject No. | 1 | 32.0 | 33.5 | 24.0 |
| | 2 | 39.0 | 38.0 | 32.0 |
| | 3 | 68.5 | 56.0 | 32.5 |
| | 4 | 53.0 | 61.0 | 75.5 |
| | 5 | 58.0 | 45.0 | 46.0 |
| | 6 | 64.5 | 50.5 | 53.0 |
| | 7 | 49.5 | 47.5 | 32.0 |
| | 8 | 32.0 | 27.0 | 23.0 |
| | 9 | 52.0 | 47.0 | 51.0 |
| | 10 | 82.5 | 59.0 | 54.5 |
| | 11 | 51.5 | 41.0 | 40.0 |
| | 12 | 30.0 | 35.0 | 36.0 |
| | 13 | 46.5 | 41.0 | 44.0 |
| | 14 | 44.5 | 49.5 | 52.5 |
| | 15 | 53.5 | 45.0 | 50.5 |

| | | | |
|---|---|---|---|
| Average of Subjects | 50.5 | 45.1 | 43.1 |
| Standard Deviation | 14.4 | 9.5 | 13.8 |
| T-test against Control Food (Probability | | 2.8% | 6.3% |
| according to t-distribution) | | | |

TABLE 5
unit: mg · min/dl

| | | | Operation | Operation |
|---|---|---|---|---|
| ΔAUC | | Control | Plot 1 | Plot 2 |
| Subject No. | 1 | 1556 | 1886 | 2074 |
| | 2 | 1808 | 1853 | 2096 |
| | 3 | 5374 | 3735 | 1973 |
| | 4 | 3998 | 4545 | 5149 |
| | 5 | 3878 | 2415 | 2861 |
| | 6 | 4673 | 3454 | 3338 |
| | 7 | 3191 | 3806 | 1706 |
| | 8 | 1594 | 1448 | 1339 |
| | 9 | 4279 | 2104 | 2963 |
| | 10 | 5220 | 4384 | 3281 |
| | 11 | 2344 | 2344 | 2355 |
| | 12 | 2449 | 1466 | 941 |
| | 13 | 2651 | 2978 | 2025 |
| | 14 | 2858 | 2561 | 4166 |
| | 15 | 4601 | 3731 | 3476 |

| | | | |
|---|---|---|---|
| Average of Subjects | 3365 | 2847 | 2650 |
| Standard Deviation | 1302 | 1034 | 1113 |
| T-test against Control Food (Probability | | 3.6% | 4.3% |
| according to t-distribution) | | | |

[Measurement Results of Blood Glucose Level]

An average of the preprandial blood glucose levels of the 15 subjects was 88.2 to 91.6 mg/dl, ΔCₘₐₓ was 45.1 mg/dl in the operation plot 1 and 43.1 mg/dl in the operation plot 2, which were obviously lower as compared with that of the control, 50.5 mg/dl. Besides, ΔAUC was 2847 mg·min/dl in the operation plot 1 and 2650 mg·min/dl in the operation plot 2, which were also obviously lower as compared with that of the control, 3365 mg·min/dl. In other words, when calcium alginate was added in a ratio of 5% or more with respect to a powder raw material, the increase of the blood glucose level was able to be effectively suppressed as compared with that in the boiled udon of the control.

### Example 2

In Example 2, comparison is made to a case where a Ca alginate suspension is simultaneously orally ingested. A case where boiled udon of the control of Example 1 was ingested was a control, and a case where the udon of the operation plot 1 was ingested was an operation plot S1. Incidentally, in 180 g of the udon sample of the operation plot 1, 270-mesh pass calcium alginate in an amount equivalent to 2.8 g was kneaded. A comparative plot 1 was a case where 2.8 g of the 270-mesh pass calcium alginate suspended in 100 ml of water was ingested and then the boiled udon of the control of Example 1 was ingested, and a comparative plot 2 was a case where the boiled udon of the control of Example 1 was ingested and then the suspension of the 270-mesh pass calcium alginate was ingested.

[Measurement Method for Blood Glucose Level]

The measurement of the blood glucose level was performed in Shimadaya Corporation. As subjects, 5 healthy males and females (2 males and 3 females) (healthy volunteers) over 20 years old were selected using a BMI reference value as an index. Udon was cooked and served in the same manner as in Example 1, and the blood glucose level was measured 30 minutes, 60 minutes, 90 minutes and 120 minutes after finishing the ingestion.

The thus measured blood glucose level is shown in [Table 6], a change in the blood glucose level is shown in [Table 7], a difference (ΔCₘₐₓ) between a maximum blood glucose level and preprandial blood glucose level is shown in [Table 8], and a product (ΔAUC) of the blood glucose level and the time calculated using the preprandial blood glucose level as a baseline is shown in [Table 9]. Besides, transition of an average of the blood glucose levels is illustrated in a graph of FIG. 2.

TABLE 6

| | | Control | | | | | Operation Plot S1 | | | | | Comparative Plot 1 | | |
|---|---|---|---|---|---|---|---|---|---|---|---|---|---|---|
| | | Time Elapsed after Ingestion | | | | | | | | | | | | |
| | | | 30 | 60 | 90 | 120 | | 30 | 60 | 90 | 120 | | 30 | 60 |
| | | Prior | min | min | min | min | Prior | min | min | min | min | Prior | min | min |
| Subject | 1 | 92.0 | 140.0 | 133.5 | 122.0 | 124.5 | 92.0 | 129.0 | 125.0 | 102.0 | 99.0 | 97.0 | 149.5 | 146.0 |
| No. | 2 | 93.0 | 142.0 | 102.0 | 111.5 | 111.0 | 88.0 | 129.0 | 99.0 | 108.0 | 90.0 | 87.5 | 117.0 | 114.0 |
| | 3 | 83.5 | 118.0 | 88.0 | 98.5 | 90.0 | 84.0 | 108.0 | 89.0 | 98.0 | 91.0 | 82.5 | 111.0 | 109.0 |
| | 4 | 101.0 | 144.0 | 112.0 | 103.0 | 107.0 | 91.0 | 126.0 | 124.0 | 111.0 | 110.0 | 92.0 | 117.5 | 109.5 |
| | 5 | 94.0 | 121.5 | 107.0 | 103.5 | 97.5 | 90.0 | 122.0 | 99.5 | 96.5 | 94.0 | 104.0 | 138.0 | 106.5 |

| | | | | | | | | | | | | | |
|---|---|---|---|---|---|---|---|---|---|---|---|---|---|
| Average of | 92.7 | 133.1 | 108.5 | 107.7 | 106.0 | 89.0 | 122.8 | 107.3 | 103.1 | 96.8 | 92.6 | 126.6 | 117.0 |
| Subjects | | | | | | | | | | | | | |

| | | | | | | |
|---|---|---|---|---|---|---|
| T-test against Control Food at Corresponding | 2.7% | 76.3% | 37.2% | 19.2% | 49.8% | 12.5% |
| Time (Probability according to t-distribution) | | | | | | |

unit: mg/dl

| | | Control | | | | | Comparative Plot 1 | | Comparative Plot 2 | | | | |
|---|---|---|---|---|---|---|---|---|---|---|---|---|---|
| | | Time Elapsed after Ingestion | | | | | | | | | | | |
| | | | 30 | 60 | 90 | 120 | 90 | 120 | | 30 | 60 | 90 | 120 |
| | | Prior | min | min | min | min | min | min | Prior | min | min | min | min |
| Subject | 1 | 92.0 | 140.0 | 133.5 | 122.0 | 124.5 | 128.0 | 124.5 | 91.0 | 131.5 | 108.0 | 90.0 | 103.0 |
| No. | 2 | 93.0 | 142.0 | 102.0 | 111.5 | 111.0 | 105.5 | 115.0 | 98.5 | 150.5 | 121.0 | 118.5 | 118.5 |
| | 3 | 83.5 | 118.0 | 88.0 | 98.5 | 90.0 | 86.0 | 86.0 | 84.5 | 132.5 | 93.5 | 88.5 | 88.0 |
| | 4 | 101.0 | 144.0 | 112.0 | 103.0 | 107.0 | 115.0 | 104.5 | 92.5 | 141.5 | 117.0 | 118.0 | 100.0 |
| | 5 | 94.0 | 121.5 | 107.0 | 103.5 | 97.5 | 118.0 | 111.5 | 97.5 | 120.5 | 110.0 | 114.5 | 106.5 |

| | | | | | | | | | | | | |
|---|---|---|---|---|---|---|---|---|---|---|---|---|
| Average of | 92.7 | 133.1 | 108.5 | 107.7 | 106.0 | 110.5 | 108.3 | 92.8 | 135.3 | 109.9 | 105.9 | 103.2 |
| Subjects | | | | | | | | | | | | |

| | | | | | | |
|---|---|---|---|---|---|---|
| T-test against Control Food at Corresponding | 62.0% | 51.5% | 62.1% | 85.7% | 84.6% | 64.0% |
| Time (Probability according to t-distribution) | | | | | | |

TABLE 7

| | | Control | | | | Operation Plot S1 | | | | Comparative Plot 1 | |
|---|---|---|---|---|---|---|---|---|---|---|---|
| | | Time Elapsed after Ingestion | | | | | | | | | |
| | | 30 | 60 | 90 | 120 | 30 | 60 | 90 | 120 | 30 | 60 |
| | | min | min | min | min | min | min | min | min | min | min |
| Subject | 1 | 48.0 | 41.5 | 30.0 | 32.5 | 37.0 | 33.0 | 10.0 | 7.0 | 52.5 | 49.0 |
| No. | 2 | 49.0 | 9.0 | 18.5 | 18.0 | 41.0 | 11.0 | 20.0 | 2.0 | 29.5 | 26.5 |
| | 3 | 34.5 | 4.5 | 15.0 | 6.5 | 24.0 | 5.0 | 14.0 | 7.0 | 28.5 | 26.5 |
| | 4 | 43.0 | 11.0 | 2.0 | 6.0 | 35.0 | 33.0 | 20.0 | 19.0 | 25.5 | 17.5 |
| | 5 | 27.5 | 13.0 | 9.5 | 3.5 | 32.0 | 9.5 | 6.5 | 4.0 | 34.0 | 2.5 |

| | | | | | | | | | | |
|---|---|---|---|---|---|---|---|---|---|---|
| Average of | 40.4 | 15.8 | 15.0 | 13.3 | 33.8 | 18.3 | 14.1 | 7.8 | 34.0 | 24.4 |
| Subjects | | | | | | | | | | |

| | | | | | | |
|---|---|---|---|---|---|---|
| T-test against Control Food at Corresponding | 8.1% | 65.6% | 88.9% | 46.4% | 30.0% | 20.0% |
| Time (Probability according to t-distribution) | | | | | | |

unit: mg/dl

| | | Control | | | | Comparative Plot 1 | | Comparative Plot 2 | | | |
|---|---|---|---|---|---|---|---|---|---|---|---|
| | | Time Elapsed after Ingestion | | | | | | | | | |
| | | 30 | 60 | 90 | 120 | 90 | 120 | 30 | 60 | 90 | 120 |
| | | min | min | min | min | min | min | min | min | min | min |
| Subject | 1 | 48.0 | 41.5 | 30.0 | 32.5 | 31.0 | 27.5 | 40.5 | 17.0 | −1.0 | 12.0 |
| No. | 2 | 49.0 | 9.0 | 18.5 | 18.0 | 18.0 | 27.5 | 52.0 | 22.5 | 20.0 | 20.0 |
| | 3 | 34.5 | 4.5 | 15.0 | 6.5 | 3.5 | 3.5 | 48.0 | 9.0 | 4.0 | 3.5 |
| | 4 | 43.0 | 11.0 | 2.0 | 6.0 | 23.0 | 12.5 | 49.0 | 24.5 | 25.5 | 7.5 |
| | 5 | 27.5 | 13.0 | 9.5 | 3.5 | 14.0 | 7.5 | 23.0 | 12.5 | 17.0 | 9.0 |

| | | | | | | | | | | |
|---|---|---|---|---|---|---|---|---|---|---|
| Average of | 40.4 | 15.8 | 15.0 | 13.3 | 17.9 | 15.7 | 42.5 | 17.1 | 13.1 | 10.4 |
| Subjects | | | | | | | | | | |

| | | | | | | |
|---|---|---|---|---|---|---|
| T-test against Control Food at Corresponding | 61.1% | 43.5% | 60.6% | 86.1% | 84.6% | 56.3% |
| Time (Probability according to t-distribution) | | | | | | |

TABLE 8
unit: mg/dl

| | | | | Compar- | Compar- |
|---|---|---|---|---|---|
| | | | Operation | ative | ative |
| ΔCₘₐₓ | | Control | Plot S1 | Plot 1 | Plot 2 |
| Subject No. | 1 | 48.0 | 37.0 | 52.5 | 40.5 |
| | 2 | 49.0 | 41.0 | 29.5 | 52.0 |
| | 3 | 34.5 | 24.0 | 28.5 | 48.0 |
| | 4 | 43.0 | 35.0 | 25.5 | 49.0 |
| | 5 | 27.5 | 32.0 | 34.0 | 23.0 |

| | | | | |
|---|---|---|---|---|
| Average of Subjects | 40.4 | 33.8 | 34.0 | 42.5 |
| Standard Deviation | 9.2 | 6.4 | 10.8 | 11.7 |
| T-test against Operation | 8.1% | | 97.0% | 19.0% |
| Plot S1 (Probability | | | | |
| according to t-distribution) | | | | |

TABLE 9
unit: mg · min/dl

| | | | | Compar- | Compar- |
|---|---|---|---|---|---|
| | | | Operation | ative | ative |
| ΔAUC | | Control | Plot S1 | Plot 1 | Plot 2 |
| Subject No. | 1 | 4073 | 2505 | 4388 | 1875 |
| | 2 | 2565 | 2190 | 2633 | 3135 |
| | 3 | 1718 | 1395 | 1808 | 1883 |
| | 4 | 1770 | 2925 | 2168 | 3083 |
| | 5 | 1553 | 1500 | 1628 | 1710 |

| | | | | |
|---|---|---|---|---|
| Average of Subjects | 2336 | 2103 | 2525 | 2337 |
| Standard Deviation | 1047 | 654 | 1110 | 708 |
| T-test against Operation | 62.1% | | 37.7% | 41.4% |
| Plot S1 (Probability | | | | |
| according to t-distribution) | | | | |

[Measurement Results of Blood Glucose Level]

As a result of the measurement of the 5 subjects, an average of the preprandial blood glucose levels of the 5 subjects was 89.0 to 92.8 mg/dl. ΔCₘₐₓ was 40.4 mg/dl in the control plot, but was 33.8 mg/dl in the operation plot S1, 34.0 mg/dl in the comparative plot 1 and 42.5 mg/dl in the comparative plot 2, and thus, the lowest value was obtained in the operation plot S1. Incidentally, it was in the control plot and the comparative plot 2 that there was an obvious difference from the operation plot S1. ΔAUC was 2336 mg·min/dl in the control plot, but 2103 mg·min/dl in the operation plot S1, 2525 mg·min/dl in the comparative plot 1 and 2337 mg·min/dl in the comparative plot 2, and thus, the lowest value was obtained in the operation plot S1. Incidentally, it was in the comparative plot 1 that there was a larger difference from the operation plot S1. Accordingly, it was confirmed that kneading of calcium alginate in udon is more effective than simultaneous oral ingestion of the same amount of calcium alginate.

### Example 3

Comparison in the effect of suppressing postprandial blood glucose level increase is made between boiled udon in which sodium alginate is added instead of calcium alginate, and boiled udon further subjected to a Ca alginate gel formation treatment.

[Preparation Method for Samples]

In a control plot and an operation plot 3 of Example 3, samples were prepared in the same manner as in Example 1 except that a wheat flour principally containing “Kitahonami” was used. In a comparative plot 3 in which Na alginate was added, and a comparative plot 4 subjected to the Ca alginate gel formation treatment, powder raw materials shown in [Table 10] were homogeneously mixed, kneading water was added thereto, and the resultant was boiled for a boiling time shown in [Table 10] after performing the noodle making step of Example 1.

Incidentally, in the comparative plot 4, etherified tapioca starch having a higher gelatinization viscosity than acetylated tapioca starch was used and its mixing amount was doubled in consideration that texture becomes hard through the Ca alginate gel formation treatment.

Boiled noodle strings were somewhat cooled by putting them in water at 15° C. twice, and were dipped in a 0.4% by mass acetic acid solution at 5° C. for 1 minute and 45 seconds in the comparative plot 3. On the other hand, in the comparative plot 4, the noodle strings were dipped in a mixture liquid of 0.3% by mass calcium chloride and 0.4% by mass acetic acid at 5° C. for 1 minute and 45 seconds to perform the calcium alginate gel formation treatment. In both the plots, the solution remaining on the noodle strings was rapidly removed, 180 g thereof corresponding to each serving was sealed with a polyethylene film, subjected to steam sterilization at 85° C. for 30 minutes, and rapidly cooled in a refrigerator at 10° C. to prepare chilled boiled udon. A boiled noodle moisture of the boiled noodles is shown in [Table 10]. Besides, the grain size of calcium alginate was 270 mesh pass.

TABLE 10

| | Sample Type | | | | | | | |
|---|---|---|---|---|---|---|---|---|
| | | | Operation | | Comparative | | Comparative | |
| Item | Control | | Plot 3 | | Plot 3 | | Plot 4 | |
| (Powder Raw Materials) | | | | | | | | |
| Wheat Flour mainly | 2760 | g | 2760 | g | 2760 | g | 2520 | g |
| composed of Kitahonami | | | | | | | | |
| Acetylated Tapioca Starch | 240 | g | 240 | g | 240 | g | 0 | g |
| Etherified Tapioca Starch | 0 | g | 0 | g | 0 | g | 480 | g |
| Calcium Alginate | 0 | g | 150 | g | 0 | g | 0 | g |
| Sodium Alginate | 0 | g | 0 | g | 150 | g | 150 | g |
| Total of Powders | 3000 | g | 3150 | g | 3240 | g | 3240 | g |
| (Kneading Water) | | | | | | | | |
| 5% Salt Solution | 0 | g | 0 | g | 1215 | g | 1215 | g |
| 10% Salt Solution | 1154 | g | 1474 | g | 0 | g | 0 | g |
| (Cooling Water) | | | | | | | | |
| 0.4% Acetic Acid | added | | added | | added | | added | |
| 0.3% Calcium Chloride | not added | | not added | | not added | | added | |
| Boiling Time | 13 | min | 13 | min | 11 | min | 11 | min |

| | | | | |
|---|---|---|---|---|
| Boiled Noodle Moisture | 67.6% | 67.3% | 67.0% | 66.8% |

[Measurement Method for Blood Glucose Level]

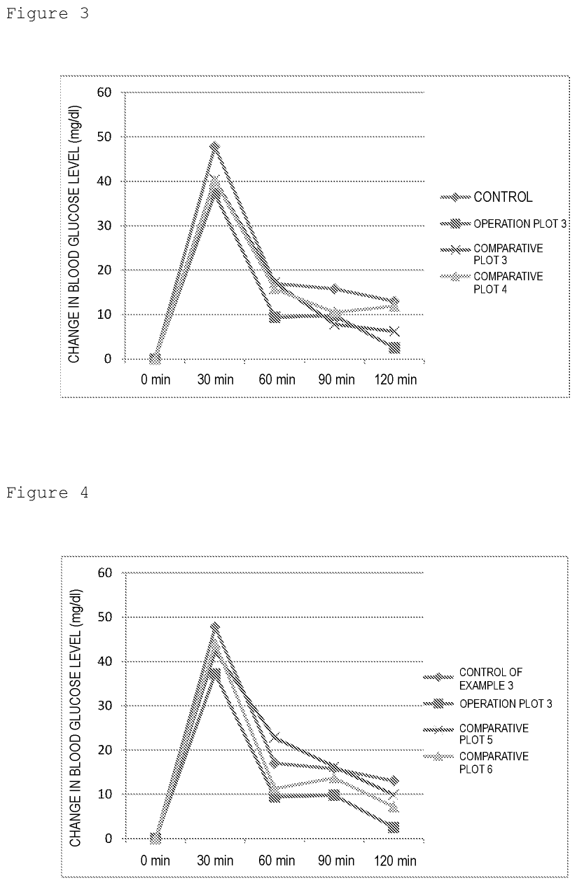

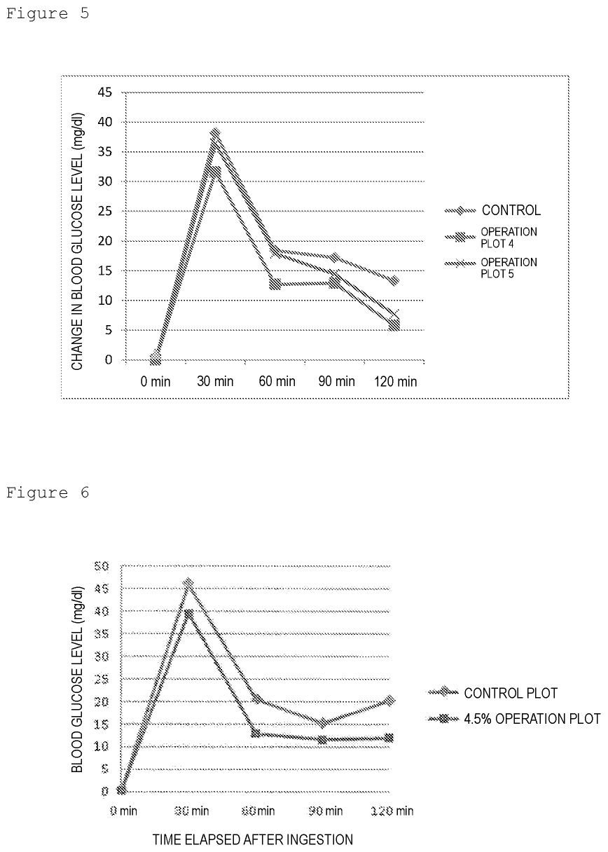

The measurement of the blood glucose level was performed in Shimadaya Corporation. Subjects were the same as those of Example 2, and cooking and serving of udon and the measurement times of the blood glucose level were the same as those of Example 2. The thus obtained blood glucose level is shown in [Table 11], a change in the blood glucose level is shown in [Table 12], a difference (ΔCₘₐₓ) between a maximum blood glucose level and preprandial blood glucose level is shown in [Table 13], and a product (ΔAUC) of the blood glucose level and the time calculated using the preprandial blood glucose level as a baseline is shown in [Table 14]. Besides, transition of an average of the blood glucose levels is illustrated in a graph of FIG. 3.

TABLE 11

| | | Control | | | | | Operation Plot 3 | | | | | Comparative Plot 3 | | |
|---|---|---|---|---|---|---|---|---|---|---|---|---|---|---|
| | | Time Elapsed after Ingestion | | | | | | | | | | | | |
| | | | 30 | 60 | 90 | 120 | | 30 | 60 | 90 | 120 | | 30 | 60 |
| | | Prior | min | min | min | min | Prior | min | min | min | min | Prior | min | min |
| Subject | 1 | 93.5 | 152.5 | 122.5 | 120.5 | 108.0 | 92.5 | 135.5 | 121.0 | 110.0 | 93.0 | 89.0 | 137.0 | 120.5 |
| No. | 2 | 92.0 | 131.0 | 92.5 | 94.5 | 106.5 | 92.0 | 132.0 | 86.5 | 102.5 | 93.5 | 85.5 | 133.0 | 106.0 |
| | 3 | 81.0 | 114.0 | 87.5 | 93.5 | 93.0 | 83.5 | 114.5 | 85.5 | 91.5 | 93.0 | 85.0 | 129.0 | 103.5 |
| | 4 | 87.5 | 157.5 | 126.0 | 105.5 | 107.5 | 87.0 | 135.0 | 101.5 | 102.0 | 87.0 | 95.0 | 133.0 | 99.5 |
| | 5 | 90.0 | 128.0 | 100.5 | 109.0 | 94.0 | 90.0 | 114.0 | 97.5 | 88.0 | 91.0 | 96.0 | 120.5 | 107.0 |

| | | | | | | | | | | | | | |
|---|---|---|---|---|---|---|---|---|---|---|---|---|---|
| Average of | 88.8 | 136.6 | 105.8 | 104.6 | 101.8 | 89.0 | 126.2 | 98.4 | 98.8 | 91.5 | 90.1 | 130.5 | 107.3 |
| Subjects | | | | | | | | | | | | | |

| | | | | | | |
|---|---|---|---|---|---|---|
| T-test against Control Food at Corresponding | 9.4% | 16.4% | 29.5% | 5.5% | 42.4% | 85.4% |
| Time (Probability according to t-distribution) | | | | | | |

unit: mg/dl

| | | Control | | | | | Comparative Plot 3 | | Comparative Plot 4 | | | | |
|---|---|---|---|---|---|---|---|---|---|---|---|---|---|
| | | Time Elapsed after Ingestion | | | | | | | | | | | |
| | | | 30 | 60 | 90 | 120 | 90 | 120 | | 30 | 60 | 90 | 120 |
| | | Prior | min | min | min | min | min | min | Prior | min | min | min | min |
| Subject | 1 | 93.5 | 152.5 | 122.5 | 120.5 | 108.0 | 102.5 | 97.0 | 90.5 | 133.0 | 113.0 | 109.0 | 103.0 |
| No. | 2 | 92.0 | 131.0 | 92.5 | 94.5 | 106.5 | 94.0 | 101.0 | 87.0 | 137.5 | 110.0 | 112.0 | 112.5 |
| | 3 | 81.0 | 114.0 | 87.5 | 93.5 | 93.0 | 94.0 | 91.0 | 84.0 | 116.0 | 91.0 | 89.5 | 102.5 |
| | 4 | 87.5 | 157.5 | 126.0 | 105.5 | 107.5 | 102.5 | 96.5 | 95.5 | 138.0 | 116.5 | 93.0 | 92.0 |
| | 5 | 90.0 | 128.0 | 100.5 | 109.0 | 94.0 | 96.5 | 96.0 | 98.0 | 131.5 | 103.5 | 104.0 | 104.5 |

| | | | | | | | | | | | | |
|---|---|---|---|---|---|---|---|---|---|---|---|---|
| Average of | 88.8 | 136.6 | 105.8 | 104.6 | 101.8 | 97.9 | 96.3 | 91.0 | 131.2 | 106.8 | 101.5 | 102.9 |
| Subjects | | | | | | | | | | | | |

| | | | | | | |
|---|---|---|---|---|---|---|
| T-test against Control Food at Corresponding | 14.0% | 9.6% | 40.5% | 85.2% | 59.8% | 83.6% |
| Time (Probability according to t-distribution) | | | | | | |

TABLE 12

| | | Control | | | | Operation Plot 3 | | | | Comparative Plot 3 | | |
|---|---|---|---|---|---|---|---|---|---|---|---|---|
| | | Time Elapsed after Ingestion | | | | | | | | | | |
| | | 30 | 60 | 90 | 120 | 30 | 60 | 90 | 120 | 30 | 60 | 90 |
| | | min | min | min | min | min | min | min | min | min | min | min |
| Subject | 1 | 59.0 | 29.0 | 27.0 | 14.5 | 43.0 | 28.5 | 17.5 | 0.5 | 48.0 | 31.5 | 13.5 |
| No. | 2 | 39.0 | 0.5 | 2.5 | 14.5 | 40.0 | −5.5 | 10.5 | 1.5 | 47.5 | 20.5 | 8.5 |
| | 3 | 33.0 | 6.5 | 12.5 | 12.0 | 31.0 | 2.0 | 8.0 | 9.5 | 44.0 | 18.5 | 9.0 |
| | 4 | 70.0 | 38.5 | 18.0 | 20.0 | 48.0 | 14.5 | 15.0 | 0.0 | 38.0 | 4.5 | 7.5 |
| | 5 | 38.0 | 10.5 | 19.0 | 4.0 | 24.0 | 7.5 | −2.0 | 1.0 | 24.5 | 11.0 | 0.5 |

| | | | | | | | | | | | |
|---|---|---|---|---|---|---|---|---|---|---|---|
| Average of | 47.8 | 17.0 | 15.8 | 13.0 | 37.2 | 9.4 | 9.8 | 2.5 | 40.4 | 17.2 | 7.8 |
| Subjects | | | | | | | | | | | |

| | | | | | | | |
|---|---|---|---|---|---|---|---|
| T-test against Control Food at Corresponding | 7.2% | 14.5% | 27.2% | 3.6% | 40.2% | 98.4% | 13.4% |
| Time (Probability according to t-distribution) | | | | | | | |

unit: mg/dl

| | | | | | | Comparative | | | | |
|---|---|---|---|---|---|---|---|---|---|---|
| | | Control | | | | Plot 3 | Comparative Plot 4 | | | |
| | | Time Elapsed after Ingestion | | | | | | | | |
| | | 30 | 60 | 90 | 120 | 120 | 30 | 60 | 90 | 120 |
| | | min | min | min | min | min | min | min | min | min |
| Subject | 1 | 59.0 | 29.0 | 27.0 | 14.5 | 8.0 | 42.5 | 22.5 | 18.5 | 12.5 |
| No. | 2 | 39.0 | 0.5 | 2.5 | 14.5 | 15.5 | 50.5 | 23.0 | 25.0 | 25.5 |
| | 3 | 33.0 | 6.5 | 12.5 | 12.0 | 6.0 | 32.0 | 7.0 | 5.5 | 18.5 |
| | 4 | 70.0 | 38.5 | 18.0 | 20.0 | 1.5 | 42.5 | 21.0 | −2.5 | −3.5 |
| | 5 | 38.0 | 10.5 | 19.0 | 4.0 | 0.0 | 33.5 | 5.5 | 6.0 | 6.5 |

| | | | | | | | | | |
|---|---|---|---|---|---|---|---|---|---|
| Average of | 47.8 | 17.0 | 15.8 | 13.0 | 6.2 | 40.2 | 15.8 | 10.5 | 11.9 |
| Subjects | | | | | | | | | |

| | | | | | |
|---|---|---|---|---|---|
| T-test against Control Food at Corresponding | 10.2% | 31.9% | 86.5% | 51.0% | 86.3% |
| Time (Probability according to t-distribution) | | | | | |

TABLE 13
unit: mg/dl

| | | | | Compar- | Compar- |
|---|---|---|---|---|---|
| | | | Operation | ative | ative |
| ΔCₘₐₓ | | Control | Plot 3 | Plot 3 | Plot 4 |
| Subject No. | 1 | 59.0 | 43.0 | 48.0 | 42.5 |
| | 2 | 39.0 | 40.0 | 47.5 | 50.5 |
| | 3 | 33.0 | 31.0 | 44.0 | 32.0 |
| | 4 | 70.0 | 48.0 | 38.0 | 42.5 |
| | 5 | 38.0 | 24.0 | 24.5 | 33.5 |

| | | | | |
|---|---|---|---|---|
| Average of Subjects | 47.8 | 37.2 | 40.4 | 40.2 |
| Standard Deviation | 15.9 | 9.6 | 9.7 | 7.6 |
| T-test against Operation | 7.2% | | 45.5% | 38.2% |
| Plot 3 (Probability | | | | |
| according to t-distribution) | | | | |

TABLE 14
unit: mg · min/dl

| | | | Operation | Comparative | Comparative |
|---|---|---|---|---|---|
| ΔAUC | | Control | Plot 3 | Plot 3 | Plot 4 |
| Subject No. | 1 | 3668 | 2678 | 2910 | 2693 |
| | 2 | 1478 | 1373 | 2528 | 3338 |
| | 3 | 1740 | 1373 | 2235 | 1613 |
| | 4 | 4095 | 2325 | 1523 | 1778 |
| | 5 | 2085 | 900 | 1080 | 1448 |

| | | | | |
|---|---|---|---|---|
| Average of Subjects | 2613 | 1730 | 2055 | 2174 |
| Standard Deviation | 1187 | 741 | 745 | 810 |
| T-test against Operation | 4.1% | | 38.9% | 35.0% |
| Plot 3 (Probability | | | | |
| according to t-distribution) | | | | |

[Measurement Results of Blood Glucose Level]

An average of the preprandial blood glucose levels of the 5 subjects was 88.8 to 91.0 mg/dl, ΔCₘₐₓ was 47.8 mg/dl in the control plot, but was 37.2 mg/dl in the operation plot 3, 40.4 mg/dl in the comparative plot 3 and 40.2 mg/dl in the comparative plot 4, and thus, was the lowest in the operation plot 3 in which calcium alginate was kneaded, and second lowest in the comparative plot 4 subjected to the calcium alginate gel formation treatment, followed by the comparative plot 3 in which sodium alginate was kneaded. ΔAUC was 2613 mg·min/dl in the control plot, but 1730 mg·min/dl in the operation plot 3, 2055 mg·min/dl in the comparative plot 3 and 2174 mg·min/dl in the comparative plot 4, and thus, was the lowest in the operation plot 3 in which calcium alginate was kneaded in the same manner as the value of ΔCₘₐₓ, and second lowest in the comparative plot in which sodium alginate was kneaded, followed by the comparative plot 4 subjected to the calcium alginate gel formation treatment.

Accordingly, it was revealed that kneading of calcium alginate suppresses the increase of the blood glucose level caused by udon more effectively than kneading of sodium alginate or the calcium gel formation treatment performed in boiled noodles in which sodium alginate is kneaded.

[Check of Viscosity of Water used for Boiling]

100 g of the noodle strings of each of the operation plot 3 and the comparative plot 3 were boiled in 1 L of water used for boiling at 98° C. for 13 minutes in a 1.5 L cooking pan, the water used for boiling was allowed to cool to 20° C. to be measured under the same conditions using a BROOKFIELD viscometer (manufactured by Eko Instruments Co., Ltd.), and as a result, the viscosity was 2.80 CP in the operation plot 3 and 3.33 CP in the comparative plot 3, and thus, sodium alginate was eluted through boiling to increase the viscosity of the water used for boiling. The results are shown in [Table 15]

TABLE 15
(Rotating Speed: 60 rpm, Spindle: LV-1)

unit: CP (mPa · S)

| | | Operation | Comparative |
|---|---|---|---|
| | | Plot 3 | Plot 3 |
| Sample No. | 1 | 2.80 | 3.30 |
| | 2 | 2.70 | 3.50 |
| | 3 | 2.90 | 3.20 |

| | | |
|---|---|---|
| Average | 2.80 | 3.33 |
| Standard Deviation | 0.10 | 0.15 |

| | |
|---|---|
| T-test | 6.7% |
| (Probability according to t-distribution) | |

### Example 4

The grain size of calcium alginate added in the operation plots 1 and 2, the operation plot S1 and the operation plot 3 was 270 mesh pass. Different grain sizes of 150 mesh pass and 80 mesh pass are compared.

[Preparation Method for Samples]

Udon samples were prepared in the same manner as in Example 3 except that an addition percentage of calcium alginate was changed to 5% by mass with respect to the powder raw material, and that a grain size of 150 mesh pass was used in a comparative plot 5 and 80 mesh pass was used in a comparative plot 6. Incidentally, data of the 270 mesh pass and the control was that obtained in Example 3.

TABLE 16

| | Test Type | | | | | | | |
|---|---|---|---|---|---|---|---|---|
| | | | Operation | | | | | |
| | Control of | | Plot 3 of | | Comparative | | Comparative | |
| Item | Example 3 | | Example 3 | | Plot 5 | | Plot 6 | |
| (Powder Raw Materials) | | | | | | | | |
| Wheat Flour mainly | 2760 | g | 2760 | g | 2760 | g | 2760 | g |
| composed of Kitahonami | | | | | | | | |
| Acetylated Tapioca Starch | 240 | g | 240 | g | 240 | g | 240 | g |
| 270 Mesh Pass Ca Alginate | 0 | g | 150 | g | 0 | g | 0 | g |
| 150 Mesh Pass Ca Alginate | 0 | g | 0 | g | 150 | g | 0 | g |
| 80 Mesh Pass Ca Alginate | 0 | g | 0 | g | 0 | g | 150 | g |
| Total of Powders | 3000 | g | 3150 | g | 3150 | g | 3150 | g |
| (Kneading Water) | | | | | | | | |
| 10% Salt Solution | 1154 | g | 1474 | g | 1474 | g | 1474 | g |
| Boiling Time | 13 | min | 13 | min | 13 | min | 13 | min |

| | | | | |
|---|---|---|---|---|
| Boiled Noodle Moisture | 67.6% | 67.3% | 67.7% | 67.4% |

[Measurement Method for Blood Glucose Level]

The measurement of the blood glucose level was performed in Shimadaya Corporation. Subjects were the same as those of Example 3, and cooking and serving of udon and the measurement times of the blood glucose level were the same as those of Example 3. The thus obtained blood glucose level is shown in [Table 17], a change in the blood glucose level is shown in [Table 18], a difference (ΔCₘₐₓ) between a maximum blood glucose level and preprandial blood glucose level is shown in [Table 19], and a product (ΔAUC) of the blood glucose level and the time calculated using the preprandial blood glucose level as a baseline is shown in [Table 20]. Besides, transition of an average of the blood glucose levels is illustrated in a graph of FIG. 4.

TABLE 17

| | | Control of | | | | | Operation Plot | | | | | Comparative | | |
|---|---|---|---|---|---|---|---|---|---|---|---|---|---|---|
| | | Example 3 | | | | | 3 of Example 3 | | | | | Plot 5 | | |
| | | Time Elapsed after Ingestion | | | | | | | | | | | | |
| | | | 30 | 60 | 90 | 120 | | 30 | 60 | 90 | 120 | | 30 | 60 |
| | | Prior | min | min | min | min | Prior | min | min | min | min | Prior | min | min |
| Subject | 1 | 93.5 | 152.5 | 122.5 | 120.5 | 108.0 | 92.5 | 135.5 | 121.0 | 110.0 | 93.0 | 95.5 | 150.0 | 133.0 |
| No. | 2 | 92.0 | 131.0 | 92.5 | 94.5 | 106.5 | 92.0 | 132.0 | 86.5 | 102.5 | 93.5 | 94.5 | 137.5 | 95.5 |
| | 3 | 81.0 | 114.0 | 87.5 | 93.5 | 93.0 | 83.5 | 114.5 | 85.5 | 91.5 | 93.0 | 85.5 | 130.5 | 111.5 |
| | 4 | 87.5 | 157.5 | 126.0 | 105.5 | 107.5 | 87.0 | 135.0 | 101.5 | 102.0 | 87.0 | 99.0 | 128.5 | 131.0 |
| | 5 | 90.0 | 128.0 | 100.5 | 109.0 | 94.0 | 90.0 | 114.0 | 97.5 | 88.0 | 91.0 | 98.5 | 138.0 | 116.0 |

| | | | | | | | | | | | | | |
|---|---|---|---|---|---|---|---|---|---|---|---|---|---|
| Average of | 88.8 | 136.6 | 105.8 | 104.6 | 101.8 | 89.0 | 126.2 | 98.4 | 98.8 | 91.5 | 94.6 | 136.9 | 117.4 |
| Subjects | | | | | | | | | | | | | |

| | | | | | | |
|---|---|---|---|---|---|---|
| T-test against Control Food at Corresponding | 9.4% | 16.4% | 29.5% | 5.5% | 97.2% | 3.8% |
| Time (Probability according to t-distribution) | | | | | | |

unit: mg/dl

| | | Control of | | | | | Comparative | | Comparative | | | | |
|---|---|---|---|---|---|---|---|---|---|---|---|---|---|
| | | Example 3 | | | | | Plot 5 | | Plot 6 | | | | |
| | | Time Elapsed after Ingestion | | | | | | | | | | | |
| | | | 30 | 60 | 90 | 120 | 90 | 120 | | 30 | 60 | 90 | 120 |
| | | Prior | min | min | min | min | min | min | Prior | min | min | min | min |
| Subject No. | 1 | 93.5 | 152.5 | 122.5 | 120.5 | 108.0 | 125.0 | 105.0 | 96.0 | 142.0 | 120.5 | 103.0 | 104.0 |
| | 2 | 92.0 | 131.0 | 92.5 | 94.5 | 106.5 | 105.5 | 110.0 | 92.0 | 134.0 | 111.5 | 113.5 | 104.5 |
| | 3 | 81.0 | 114.0 | 87.5 | 93.5 | 93.0 | 91.5 | 96.0 | 85.0 | 128.5 | 92.0 | 104.5 | 87.0 |
| | 4 | 87.5 | 157.5 | 126.0 | 105.5 | 107.5 | 122.5 | 105.5 | 92.5 | 147.5 | 94.5 | 96.0 | 103.5 |
| | 5 | 90.0 | 128.0 | 100.5 | 109.0 | 94.0 | 109.5 | 106.0 | 102.0 | 136.0 | 105.5 | 119.0 | 104.5 |

| | | | | | | | | | | | | |
|---|---|---|---|---|---|---|---|---|---|---|---|---|
| Average of | 88.8 | 136.6 | 105.8 | 104.6 | 101.8 | 110.8 | 104.5 | 93.5 | 137.6 | 104.8 | 107.2 | 100.7 |
| Subjects | | | | | | | | | | | | |

| | | | | | | |
|---|---|---|---|---|---|---|
| T-test against Control Food at Corresponding | 14.9% | 36.8% | 85.0% | 91.1% | 72.4% | 73.0% |
| Time (Probability according to t-distribution) | | | | | | |

TABLE 18

| | | Control of | | | | Operation Plot | | | | | Comparative |
|---|---|---|---|---|---|---|---|---|---|---|---|
| | | Example 3 | | | | 3 of Example 3 | | | | | Plot 5 |
| | | Time Elapsed after Ingestion | | | | | | | | | |
| | | 30 | 60 | 90 | 120 | 30 | 60 | 90 | 120 | 30 | 60 |
| | | min | min | min | min | min | min | min | min | min | min |
| Subject | 1 | 59.0 | 29.0 | 27.0 | 14.5 | 43.0 | 28.5 | 17.5 | 0.5 | 54.5 | 37.5 |
| No. | 2 | 39.0 | 0.5 | 2.5 | 14.5 | 40.0 | −5.5 | 10.5 | 1.5 | 43.0 | 1.0 |
| | 3 | 33.0 | 6.5 | 12.5 | 12.0 | 31.0 | 2.0 | 8.0 | 9.5 | 45.0 | 26.0 |
| | 4 | 70.0 | 38.5 | 18.0 | 20.0 | 48.0 | 14.5 | 15.0 | 0.0 | 29.5 | 32.0 |
| | 5 | 38.0 | 10.5 | 19.0 | 4.0 | 24.0 | 7.5 | −2.0 | 1.0 | 39.5 | 17.5 |

| | | | | | | | | | | |
|---|---|---|---|---|---|---|---|---|---|---|
| Average of | 47.8 | 17.0 | 15.8 | 13.0 | 37.2 | 9.4 | 9.8 | 2.5 | 42.3 | 22.8 |
| Subjects | | | | | | | | | | |

| | | | | | | |
|---|---|---|---|---|---|---|
| T-test against Control Food at Corresponding | 7.2% | 14.5% | 27.2% | 3.6% | 58.0% | 25.2% |
| Time (Probability according to t-distribution) | | | | | | |

unit: mg/dl

| | | Control of | | | | Comparative | | Comparative | | | |
|---|---|---|---|---|---|---|---|---|---|---|---|
| | | Example 3 | | | | Plot 5 | | Plot 6 | | | |
| | | Time Elapsed after Ingestion | | | | | | | | | |
| | | 30 | 60 | 90 | 120 | 90 | 120 | 30 | 60 | 90 | 120 |
| | | min | min | min | min | min | min | min | min | min | min |
| Subject | 1 | 59.0 | 29.0 | 27.0 | 14.5 | 29.5 | 9.5 | 46.0 | 24.5 | 7.0 | 8.0 |
| No. | 2 | 39.0 | 0.5 | 2.5 | 14.5 | 11.0 | 15.5 | 42.0 | 19.5 | 21.5 | 12.5 |
| | 3 | 33.0 | 6.5 | 12.5 | 12.0 | 6.0 | 10.5 | 43.5 | 7.0 | 19.5 | 2.0 |
| | 4 | 70.0 | 38.5 | 18.0 | 20.0 | 23.5 | 6.5 | 55.0 | 2.0 | 3.5 | 11.0 |
| | 5 | 38.0 | 10.5 | 19.0 | 4.0 | 11.0 | 7.5 | 34.0 | 3.5 | 17.0 | 2.5 |

| | | | | | | | | | | |
|---|---|---|---|---|---|---|---|---|---|---|
| Average of | 47.8 | 17.0 | 15.8 | 13.0 | 16.2 | 9.9 | 44.1 | 11.3 | 13.7 | 7.2 |
| Subjects | | | | | | | | | | |

| | | | | | | |
|---|---|---|---|---|---|---|
| T-test against Control Food at Corresponding | 90.9% | 35.3% | 48.4% | 55.9% | 78.1% | 3.0% |
| Time (Probability according to t-distribution) | | | | | | |

TABLE 19
unit: mg/dl

| | | | | Compar- | Compar- |
|---|---|---|---|---|---|
| | | | Operation | ative | ative |
| ΔCₘₐₓ | | Control | Plot 3 | Plot 5 | Plot 6 |
| Subject No. | 1 | 59.0 | 43.0 | 54.5 | 46.0 |
| | 2 | 39.0 | 40.0 | 43.0 | 42.0 |
| | 3 | 33.0 | 31.0 | 45.0 | 43.5 |
| | 4 | 70.0 | 48.0 | 32.0 | 55.0 |
| | 5 | 38.0 | 24.0 | 39.5 | 34.0 |

| | | | | |
|---|---|---|---|---|
| Average of Subjects | 47.8 | 37.2 | 42.8 | 44.1 |
| Standard Deviation | 15.9 | 9.6 | 8.2 | 7.6 |
| T-test against Control Food | | 7.2% | 59.5% | 48.4% |
| (Probability according to t- | | | | |
| distribution) | | | | |

TABLE 20
unit: mg · min/dl

| | | | | Compar- | Compar- |
|---|---|---|---|---|---|
| | | | Operation | ative | ative |
| ΔAUC | | Control | Plot 3 | Plot 5 | Plot 6 |
| Subject No. | 1 | 3668 | 2678 | 3788 | 2445 |
| | 2 | 1478 | 1373 | 1883 | 2678 |
| | 3 | 1740 | 1373 | 2468 | 2130 |
| | 4 | 4095 | 2325 | 2648 | 1980 |
| | 5 | 2085 | 900 | 2153 | 1673 |

| | | | | |
|---|---|---|---|---|
| Average of Subjects | 2613 | 1730 | 2588 | 2181 |
| Standard Deviation | 1187 | 741 | 732 | 393 |
| T-test against Control Food | | 4.1% | 94.9% | 50.0% |
| (Probability according to t- | | | | |
| distribution) | | | | |

[Measurement Results of Blood Glucose Level]

An average of the preprandial blood glucose levels of the 5 subjects was 88.8 to 94.6 mg/dl, and ΔCₘₐₓ was 47.8 mg/dl in the control plot, and was 42.8 mg/dl in the comparative plot 5 and 44.1 mg/dl in the comparative plot 6, which were not largely different, and no significant difference was found among these. ΔAUC was 2613 mg·min/dl in the control plot, and was 2588 mg·min/dl in the comparative plot 5 and 2181 mg·min/dl in the comparative plot 6, which were not largely different, and no significant difference was not found also among these.

In other words, when calcium alginate is simply powdered, the effect of suppressing the postprandial blood glucose level increase cannot be obtained by kneading it in udon, and it was revealed that the effect can be obtained merely when it is pulverized to a grain size of about 270 mesh pass.

[Physical Property Test using Texture Analyzer]

As a sample for a physical property test using a texture analyzer, a sample corresponding to each serving stored at 10° C. for 3 days was boiled for 3 minutes in a 1.5 L cooking pan, then cooled to a temperature of noodle strings of 15° C. in 1 minute, and drained with a strainer, and measurement was performed after 1 minute from the draining every 5 minutes ten times in total. Measurement conditions were those of a 2-byte method. In the 2-byte method, in first compression, noodle strings are compressed with a plunger by 50% of their thickness to record a change of stress, the plunger is released once, and the noodle strings are compressed in the same position by 97% of the thickness of the noodle strings to record a change of the stress. The stress obtained in the compression performed twice is regarded as an index of the hardness of the noodle strings, and a change ratio of an area under a curve of the first compression and an area under a curve of the second compression corresponding to “stress×compression amount” was regarded as an index of elasticity. An elevating speed of the plunger was 1 mm per second, and the load was recorded at intervals of 0.01 seconds. The obtained results are shown in [Table 21].

TABLE 21

| | | unit: N | | | | unit: N | | | | unit: % | | | |
|---|---|---|---|---|---|---|---|---|---|---|---|---|---|
| | | Hardness (50% Compression) | | | | Hardness (97% Compression) | | | | Elasticity | | | |
| | | | | Compar- | Compar- | | | Compar- | Compar- | | | Compar- | Compar- |
| | | | Operation | ative | ative | | Operation | ative | ative | | Operation | ative | ative |
| | | Control | Plot 3 | Plot 5 | Plot 6 | Control | Plot 3 | Plot 5 | Plot 6 | Control | Plot 3 | Plot 5 | Plot 6 |
| Sample | 1 | 0.33 | 0.32 | 0.35 | 0.33 | 0.58 | 0.61 | 0.52 | 0.49 | 62.3 | 67.0 | 60.9 | 58.7 |
| No. | 2 | 0.33 | 0.37 | 0.37 | 0.32 | 0.57 | 0.64 | 0.57 | 0.47 | 63.5 | 65.7 | 62.4 | 60.1 |
| | 3 | 0.37 | 0.34 | 0.33 | 0.36 | 0.56 | 0.63 | 0.49 | 0.57 | 65.4 | 64.9 | 61.6 | 63.7 |
| | 4 | 0.34 | 0.37 | 0.33 | 0.33 | 0.55 | 0.61 | 0.51 | 0.49 | 64.0 | 64.3 | 59.0 | 53.5 |
| | 5 | 0.33 | 0.38 | 0.32 | 0.36 | 0.60 | 0.62 | 0.52 | 0.59 | 61.1 | 64.4 | 62.3 | 62.6 |
| | 6 | 0.34 | 0.35 | 0.32 | 0.33 | 0.55 | 0.56 | 0.49 | 0.51 | 64.1 | 63.6 | 61.4 | 57.4 |
| | 7 | 0.32 | 0.36 | 0.34 | 0.32 | 0.58 | 0.66 | 0.52 | 0.48 | 61.0 | 66.1 | 59.5 | 58.5 |
| | 8 | 0.33 | 0.33 | 0.34 | 0.34 | 0.58 | 0.53 | 0.50 | 0.50 | 62.3 | 62.6 | 59.6 | 58.8 |
| | 9 | 0.29 | 0.31 | 0.32 | 0.29 | 0.56 | 0.47 | 0.46 | 0.42 | 60.9 | 60.2 | 57.0 | 56.9 |
| | 10 | 0.35 | 0.32 | 0.33 | 0.36 | 0.53 | 0.47 | 0.48 | 0.53 | 64.4 | 62.0 | 56.6 | 57.3 |

| | | | | | | | | | | | | |
|---|---|---|---|---|---|---|---|---|---|---|---|---|
| Average | 0.33 | 0.34 | 0.33 | 0.34 | 0.57 | 0.58 | 0.50 | 0.51 | 62.9 | 64.1 | 60.0 | 58.8 |
| Standard | 0.02 | 0.02 | 0.01 | 0.02 | 0.02 | 0.07 | 0.03 | 0.05 | 1.59 | 2.05 | 2.06 | 2.88 |
| Deviation | | | | | | | | | | | | |

| | | | | | | | | | |
|---|---|---|---|---|---|---|---|---|---|
| T-test against | 21.2% | 92.4% | 64.2% | 53.2% | 0.0% | 0.3% | 17.0% | 0.5% | 0.3% |
| Control Food | | | | | | | | | |
| (Probability | | | | | | | | | |
| according to | | | | | | | | | |
| t-distribution) | | | | | | | | | |

As a result, the stress at the time of 97% compression was 0.57 N in the control plot, but was 0.58 N in the operation plot 3, 0.50 N in the comparative plot 5 and 0.51 N in the comparative plot 6, and thus it was revealed that merely the operation plot 3 in which the 270-mesh pass calcium alginate was added was equivalent to the control plot, and that the comparative plots 5 and 6 were significantly soft. The index value of the elasticity was 62.9% in the control plot, but was 64.1% in the operation plot 3, 60.0% in the comparative plot 5 and 58.8% in the comparative plot 6, and thus, it was revealed that the elasticity was significantly low in the comparative plots 5 and 6, and that the elasticity was not deteriorated in the operation plot 3. In other words, it was clarified that the deterioration of texture otherwise caused when it is kneaded in udon can be prevented by pulverizing calcium alginate to a grain size of about 270 mesh pass.

[Sensory Evaluation Test]

The 4 samples having been evaluated in the physical property test using a texture analyzer were subjected to sensory evaluation. As an evaluation method, a 5-point rating scale assuming that the boiled udon of the control plot had a score of 3 was employed, and four items of hardness (hard: 5 to soft: 1), chewy texture (chewy: 5 to brittle: 1), powderiness (powdery: 5 to not powdery: 1), and comprehensive texture (preferable: 5 to not preferable: 1) were evaluated, and an average of scores of 8 specialized panelists was employed.

TABLE 22

| | | Hardness | | | | Chewy Texture | | | | |
|---|---|---|---|---|---|---|---|---|---|---|
| | | | Operation | Comparative | Comparative | | Operation | Comparative | Comparative | Powderiness |
| | | Control | Plot 3 | Plot 5 | Plot 6 | Control | Plot 3 | Plot 5 | Plot 6 | Control |
| Evaluator No. | 1 | 3.0 | 3.5 | 2.6 | 2.1 | 3.0 | 2.5 | 2.0 | 2.0 | 3.0 |
| | 2 | 3.0 | 3.5 | 1.6 | 2.1 | 3.0 | 2.5 | 1.5 | 2.0 | 3.0 |
| | 3 | 3.0 | 3.0 | 3.7 | 2.1 | 3.0 | 3.0 | 2.6 | 2.0 | 3.0 |
| | 4 | 3.0 | 3.0 | 3.1 | 4.2 | 3.0 | 3.0 | 2.0 | 3.1 | 3.0 |
| | 5 | 3.0 | 3.0 | 2.6 | 2.1 | 3.0 | 3.0 | 2.6 | 2.0 | 3.0 |
| | 6 | 3.0 | 3.0 | 2.1 | 4.2 | 3.0 | 3.5 | 3.6 | 3.1 | 3.0 |
| | 7 | 3.0 | 3.0 | 3.7 | 3.1 | 3.0 | 3.5 | 2.6 | 3.1 | 3.0 |
| | 8 | 3.0 | 3.0 | 2.6 | 2.6 | 3.0 | 3.5 | 3.1 | 3.1 | 3.0 |

| | | | | | | | | | |
|---|---|---|---|---|---|---|---|---|---|
| Average | 3.0 | 3.1 | 2.7 | 2.8 | 3.0 | 3.1 | 2.5 | 2.6 | 3.0 |
| Standard | | 0.2 | 0.7 | 0.9 | | 0.4 | 0.6 | 0.5 | |
| Deviation | | | | | | | | | |

| | | | | | | |
|---|---|---|---|---|---|---|
| T-test against | 17.0% | 34.2% | 56.7% | 68% | 5.6% | 5.2% |
| Control Food | | | | | | |
| (Probability | | | | | | |
| according to t- | | | | | | |
| distribution) | | | | | | |

| | | | Powderiness | | | Comprehensive Texture | | | |
|---|---|---|---|---|---|---|---|---|---|
| | | Hardness | Operation | Comparative | Comparative | | Operation | Comparative | Comparative |
| | | Control | Plot 3 | Plot 5 | Plot 6 | Control | Plot 3 | Plot 5 | Plot 6 |
| Evaluator No. | 1 | 3.0 | 3.0 | 4.1 | 4.6 | 3.0 | 3.0 | 1.9 | 1.5 |
| | 2 | 3.0 | 3.0 | 4.6 | 4.1 | 3.0 | 2.5 | 1.5 | 1.9 |
| | 3 | 3.0 | 3.0 | 4.1 | 4.6 | 3.0 | 3.0 | 1.9 | 1.5 |
| | 4 | 3.0 | 3.5 | 4.1 | 4.6 | 3.0 | 3.0 | 1.9 | 1.9 |
| | 5 | 3.0 | 3.0 | 4.1 | 4.6 | 3.0 | 3.0 | 1.9 | 1.5 |
| | 6 | 3.0 | 2.5 | 2.6 | 3.6 | 3.0 | 3.5 | 2.4 | 3.4 |
| | 7 | 3.0 | 4.0 | 4.1 | 3.6 | 3.0 | 2.5 | 1.9 | 2.4 |
| | 8 | 3.0 | 3.0 | 3.1 | 3.6 | 3.0 | 3.0 | 2.9 | 2.4 |

| | | | | | | | | |
|---|---|---|---|---|---|---|---|---|
| Average | 3.0 | 3.1 | 3.9 | 4.2 | 3.0 | 2.9 | 2.1 | 2.1 |
| Standard | | 0.4 | 0.7 | 0.5 | | 0.3 | 0.4 | 0.7 |
| Deviation | | | | | | | | |

| | | | | | | |
|---|---|---|---|---|---|---|
| T-test against | 45.1% | 0.8% | 0.0% | 59.8% | 0.0% | 0.5% |
| Control Food | | | | | | |
| (Probability | | | | | | |
| according to t- | | | | | | |
| distribution) | | | | | | |

As a result, in the item of hardness, as compared with the reference score of 3 of the control plot, the operation plot 3 had a score of 3.1, the comparative plot 5 had a score of 2.7 and the comparative plot 6 had a score of 2.8, and thus, the operation plot 3 had an equivalent value, and the comparative plots 5 and 6 had lower values. In the item of chewy texture, as compared with the reference score of 3 of the control plot, the operation plot 3 had a score of 3.1, the comparative plot 5 had a score of 2.5 and the comparative plot 6 had a score of 2.6, and thus, the operation plot 3 had an equivalent value, and the comparative plots 5 and 6 had lower values. In the item of powderiness, as compared with the reference score of 3 of the control plot, the operation plot 3 had a score of 3.1, the comparative plot 5 had a score of 3.9 and the comparative plot 6 had a score of 4.2, and thus, the operation plot 3 had an equivalent value, and the comparative plots 5 and 6 had obviously higher values. In the item of comprehensive texture, as compared with the reference score of 3 of the control plot, the operation plot 3 had a score of 2.9, the comparative plot 5 had a score of 2.1 and the comparative plot 6 had a score of 2.1, and thus, the operation plot 3 had an equivalent value, and the comparative plots 5 and 6 had obviously lower values.

In this manner, also in the sensory evaluation, it was clarified that the deterioration of texture otherwise caused when it is kneaded in udon can be prevented by pulverizing calcium alginate to a grain size of about 270 mesh pass.

### Example 5

The effect of suppressing postprandial blood glucose level increase of udon in which 5% or 4% of 270-mesh pass Ca alginate is added is checked.

[Preparation Method for Samples]

Powder raw materials shown in [Table 23] were respectively homogeneously mixed, kneading water shown in [Table 23] was respectively added thereto, and then chilled boiled udon was prepared in the same manner as in Example 1.

TABLE 23

| | Test Type | | | | | |
|---|---|---|---|---|---|---|
| | | | Operation | | Operation | |
| Item | Control | | Plot 4 | | Plot 5 | |
| (Powder Raw Materials) | | | | | | |
| Wheat Flour mainly | 2520 | g | 2520 | g | 2520 | g |
| composed of ASW | | | | | | |
| Wheat Flour mainly | 0 | g | 0 | g | 0 | g |
| composed of Kitahonami | | | | | | |
| Acetylated Tapioca Starch | 480 | g | 480 | g | 480 | g |
| Calcium Alginate | 0 | g | 150 | g | 120 | g |
| Total of Powders | 3000 | g | 3150 | g | 3120 | g |
| (Kneading Water) | | | | | | |
| 10% Salt Solution | 1298 | g | 1650 | g | 1570 | g |
| Boiling Time | 13 | min | 13 | min | 13 | min |

| | | | |
|---|---|---|---|
| Boiled Noodle Moisture | 67.4% | 67.4% | 67.6% |

[Measurement Method for Blood Glucose Level]

The measurement of the blood glucose level was performed in Shimadaya Corporation. Subjects were the same as those of Example 3, and cooking and serving of udon and the measurement times of the blood glucose level were the same as those of Example 3. The thus obtained blood glucose level is shown in [Table 24], a change in the blood glucose level is shown in [Table 25], a difference (ΔCₘₐₓ) between a maximum blood glucose level and preprandial blood glucose level is shown in [Table 26], and a product (ΔAUC) of the blood glucose level and the time calculated using the preprandial blood glucose level as a baseline is shown in [Table 27]. Besides, transition of an average of change in the blood glucose levels is illustrated in a graph of Figure

TABLE 24
unit: mg/dl

| | | Control | | | | | Operation Plot 4 | | | | | Operation Plot 5 | | | | |
|---|---|---|---|---|---|---|---|---|---|---|---|---|---|---|---|---|
| | | Time Elapsed after Ingestion | | | | | | | | | | | | | | |
| | | Prior | 30 min | 60 min | 90 min | 120 min | Prior | 30 min | 60 min | 90 min | 120 min | Prior | 30 min | 60 min | 90 min | 120 min |
| Subject | 1 | 92.0 | 140.0 | 133.5 | 122.0 | 124.5 | 92.0 | 129.0 | 125.0 | 102.0 | 99.0 | 90.0 | 134.0 | 116.0 | 115.0 | 96.5 |
| No. | 2 | 93.0 | 142.0 | 102.0 | 111.5 | 111.0 | 88.0 | 129.0 | 99.0 | 108.0 | 90.0 | 99.0 | 137.5 | 106.0 | 97.5 | 96.5 |
| | 3 | 83.5 | 118.0 | 88.0 | 98.5 | 90.0 | 84.0 | 108.0 | 89.0 | 98.0 | 91.0 | 94.5 | 133.5 | 117.5 | 114.0 | 107.5 |
| | 4 | 91.0 | 123.0 | 115.0 | 104.0 | 97.0 | 93.0 | 117.0 | 98.0 | 107.0 | 102.0 | 87.0 | 116.0 | 109.5 | 105.0 | 99.0 |
| | 5 | 94.0 | 121.5 | 107.0 | 103.5 | 97.5 | 90.0 | 122.0 | 99.5 | 96.5 | 94.0 | 89.0 | 120.5 | 100.0 | 100.0 | 98.5 |

| | | | | | | | | | | | | | | | |
|---|---|---|---|---|---|---|---|---|---|---|---|---|---|---|---|
| Average of | 90.7 | 128.9 | 109.1 | 107.9 | 104.0 | 89.4 | 121.0 | 102.1 | 102.3 | 95.2 | 91.9 | 128.3 | 109.8 | 106.3 | 99.6 |
| Subjects | | | | | | | | | | | | | | | |

| | | | | | | | | |
|---|---|---|---|---|---|---|---|---|
| T-test against Control Food at Corresponding | 3.0% | 8.1% | 23.0% | 22.2% | 89.2% | 93.4% | 76.2% | 60.2% |
| Time (Probability according to t-distribution) | | | | | | | | |

TABLE 25
unit: mg/dl

| | | Control | | | | Operation Plot 4 | | | | Operation Plot 5 | | | |
|---|---|---|---|---|---|---|---|---|---|---|---|---|---|
| | | Time Elapsed after Ingestion | | | | | | | | | | | |
| | | 30 min | 60 min | 90 min | 120 min | 30min | 60min | 90min | 120min | 30min | 60min | 90min | 120min |
| Subject | 1 | 48.0 | 41.5 | 30.0 | 32.5 | 37.0 | 33.0 | 10.0 | 7.0 | 44.0 | 26.0 | 25.0 | 6.5 |
| No. | 2 | 49.0 | 9.0 | 18.5 | 18.0 | 41.0 | 11.0 | 20.0 | 2.0 | 38.5 | 7.0 | −1.5 | −2.5 |
| | 3 | 34.5 | 4.5 | 15.0 | 6.5 | 24.0 | 5.0 | 14.0 | 7.0 | 39.0 | 23.0 | 19.5 | 13.0 |
| | 4 | 32.0 | 24.0 | 13.0 | 6.0 | 24.0 | 5.0 | 14.0 | 9.0 | 29.0 | 22.5 | 18.0 | 12.0 |
| | 5 | 27.5 | 13.0 | 9.5 | 3.5 | 32.0 | 9.5 | 6.5 | 4.0 | 31.5 | 11.0 | 11.0 | 9.5 |

| | | | | | | | | | | | | |
|---|---|---|---|---|---|---|---|---|---|---|---|---|
| Average of | 38.2 | 18.4 | 17.2 | 13.3 | 31.6 | 12.7 | 12.9 | 5.8 | 36.4 | 17.9 | 14.4 | 7.7 |
| Subjects | | | | | | | | | | | | |

| | | | | | | | | |
|---|---|---|---|---|---|---|---|---|
| T-test against Control Food at Corresponding | 8.1% | 20.7% | 34.3% | 25.4% | 55.3% | 93.1% | 58.0% | 48.3% |
| Time (Probability according to t-distribution) | | | | | | | | |

TABLE 26
unit: mg/dl

| | | | Operation | Operation |
|---|---|---|---|---|
| ΔCₘₐₓ | | Control | Plot 4 | Plot 5 |
| Subject No. | 1 | 48.0 | 37.0 | 44.0 |
| | 2 | 49.0 | 41.0 | 38.5 |
| | 3 | 34.5 | 24.0 | 39.0 |
| | 4 | 32.0 | 24.0 | 29.0 |
| | 5 | 27.5 | 32.0 | 31.5 |

| | | | |
|---|---|---|---|
| Average of Subjects | 38.2 | 31.6 | 36.4 |
| Standard Deviation | 9.7 | 7.6 | 6.1 |
| T-test against Control Food | | 8.1% | 55.3% |
| (Probability according to t- | | | |
| distribution) | | | |

TABLE 27
unit: mg · min/dl

| | | | Operation | Operation |
|---|---|---|---|---|
| Δ AUC | | Control | Plot 4 | Plot 5 |
| Subject No. | 1 | 4073 | 2505 | 2948 |
| | 2 | 2565 | 2190 | 1283 |
| | 3 | 1718 | 1395 | 2640 |
| | 4 | 2160 | 1425 | 2265 |
| | 5 | 1553 | 1500 | 1748 |

| | | | |
|---|---|---|---|
| Average of Subjects | 2414 | 1803 | 2177 |
| Standard Deviation | 1008 | 511 | 671 |
| T-test against Control Food | | 8.1% | 60.3% |
| (Probability according to t- | | | |
| distribution) | | | |

[Measurement Results of Blood Glucose Level]

An average of the preprandial blood glucose levels of the 5 subjects was 89.4 to 91.9 mg/dl, and ΔCₘₐₓ was 38.2 mg/dl in the control plot, but was 31.6 mg/dl in the operation plot 4 and 36.4 mg/dl in the operation plot 5, and thus, there was a tendency depending on a difference in the addition concentration of calcium alginate, but while there was an obvious difference in the operation plot 4 of 5% addition, there was a slight difference in the operation plot 5 of 4% addition. ΔAUC was 2414 mg·min/dl in the control plot, but was 1803 mg·min/dl in the operation plot 4 and 2177 mg·min/dl in the operation plot 5, and thus, there was a tendency depending on the difference in the addition concentration similarly to ΔCₘₐₓ, but while there was an obvious difference in the operation plot 4 of 5% addition, there was a slight difference in the operation plot 5 of 4% addition. Accordingly, regarding the effect of suppressing the blood glucose level increase obtained when 270-mesh pass powdered calcium alginate is added to a powder raw material, it was confirmed that the effect of suppressing the blood glucose level increase was obtained by 4% addition, but a more preferable effect of suppressing the blood glucose level increase is obtained by 5% addition.

### Example 6

<Addition of 4.5% by mass of Ca Alginate to Powder Raw Material for Noodle Production>: An effect of suppressing the postprandial blood glucose level increase of boiled Japanese soba (buckwheat noodles) in which 4.5% of 270 mesh pass Ca alginate was added was checked.

[Preparation of Boiled Buckwheat Noodle Samples]

Powder raw materials shown in [Table 28] were respectively homogeneously mixed, and kneading water was respectively added thereto, followed by mixing. As the powder raw material, a wheat flour and a buckwheat flour (crude protein: 11.5%, ash: 2.0%) were mixed with modified starch and vital gluten, so as to improve texture and enforce binding of a dough. 270 mesh pass Ca alginate was added in a ratio of 4.5% in an operation plot, and was not added in a comparative plot, and glycine was dissolved in the kneading water for purposes of improving the preserving property. The mixing was performed by kneading at a decompression degree of 80 kPa for 8 minutes to obtain a kneaded dough in a crumbled state at a temperature of 28 to 34° C.

The kneaded dough was formed into crude noodle belts each having a thickness of 6 mm using a noodle belt forming roll to be compounded with the thickness kept, the resultant was allowed to stand at 25° C. for 60 minutes under a condition where it was not dried, was rolled into a thickness of 1.50 mm using a four-stage flat roll, and then finely cut into noodle strings using cutting teeth with a grove width of 1.54 mm. The thus obtained noodle strings were boiled in hot water for boiling at 98° C. for 1 minute, then somewhat cooled by putting them in water at 15° C. twice, and dipped in a 0.4% by mass acetic acid solution at 5° C. for 2 minutes and 20 seconds. Thereafter, the solution remaining thereon was rapidly removed, 160 g thereof corresponding to each serving was sealed with a polyethylene film, subjected to steam sterilization at 85° C. for 30 minutes, and rapidly cooled in a refrigerator at 10° C. to prepare chilled boiled buckwheat noodles having a boiled noodle moisture shown in [Table 28].

TABLE 28

| | Sample Type | | | |
|---|---|---|---|---|
| | | | 4.5% | |
| Item | Control Plot | | Operation Plot | |
| (Powder Raw Materials) | | | | |
| Strong Wheat Flour | 1972 | g | 1972 | g |
| Buckwheat Flour | 1232 | g | 1232 | g |
| Modified Starch for buckwheat | 295.8 | g | 295.8 | g |
| noodles | | | | |
| Vital Gluten | 350 | g | 350 | g |
| Dried Egg White Powder | 26.3 | g | 35 | g |
| Total of Powder Raw Materials | 3876.1 | g | 3884.8 | g (100%) |
| For Noodle Production | | | | |
| 270 Mesh Pass Ca Alginate | | | 175 | g (4.5%) |
| Total of Powders | 3876.1 | g | 4059.8 | g |
| (Kneading Water) | | | | |
| Glycine | 41.3 | g | 41.3 | g |
| Fresh Water | 1278 | g | 1540 | g |
| Boiling Time | 1 | min | 1 | min |

| | | |
|---|---|---|
| * Boiled Noodle Average Moisture | 61.2% | 60.7% |

[Measurement Method for Blood Glucose Level]

The blood glucose level was compared with that of a control plot. The measurement of the blood glucose level was performed in Shimadaya Corporation. Subjects were 5 healthy males and females over 20 years old including a person having a fasting blood glucose level corresponding to borderline diabetes. For cooking, 160 g corresponding to each serving of each sample was boiled for 1 minute in boiling water to be served as kake soba (buckwheat noodles in a hot soup) with no ingredients. Conditions were adjusted so that the buckwheat noodles were able to be finished in 5 minutes after serving, and the blood glucose level was measured 30 minutes, 60 minutes, 90 minutes and 120 minutes after starting the ingestion.

The thus obtained blood glucose level is shown in [Table 29], a change in the blood glucose level is shown in [Table 30], a difference (ΔCₘₐₓ) between a maximum blood glucose level and preprandial blood glucose level is shown in [Table 31], and a product (ΔAUC) of the blood glucose level and the time calculated using the preprandial blood glucose level as a baseline is shown in [Table 32]. Besides, transition of an average of the blood glucose levels is illustrated in a graph of FIG. 6.

TABLE 29
unit: mg/dl

| | | Control Plot | | | | | 4.5% Operation Plot | | | | |
|---|---|---|---|---|---|---|---|---|---|---|---|
| | | Time Elapsed after Ingestion | | | | | | | | | |
| | | Prior | 30 min | 60 min | 90 min | 120 min | Prior | 30 min | 60 min | 90 min | 120 min |
| Subject | 1 | 85.0 | 122.5 | 111.0 | 105.5 | 104.0 | 82.5 | 136.5 | 97.5 | 96.5 | 91.5 |
| No. | 2 | 84.0 | 131.0 | 101.0 | 99.0 | 106.0 | 93.5 | 127.0 | 107.5 | 103.5 | 108.0 |
| | 3 | 81.5 | 158.0 | 95.0 | 109.0 | 119.0 | 88.0 | 147.5 | 96.5 | 105.0 | 101.5 |
| | 4 | 82.5 | 116.5 | 111.0 | 86.0 | 88.0 | 80.5 | 106.5 | 105.5 | 97.0 | 89.0 |
| | 5 | 99.0 | 134.0 | 116.5 | 108.5 | 116.0 | 103.0 | 128.0 | 105.0 | 104.0 | 116.5 |

| | | | | | | | | | | |
|---|---|---|---|---|---|---|---|---|---|---|
| Average of | 86.4 | 132.4 | 106.9 | 101.6 | 106.6 | 89.5 | 129.1 | 102.4 | 101.2 | 101.3 |
| Subjects | | | | | | | | | | |

| | | | | | |
|---|---|---|---|---|---|
| T-test against Control Plot | 26% | 50% | 30% | 92% | 26% |
| (Probability according to t-distribution) | | | | | |

TABLE 30
unit: m/dl

| | | Control Plot | | | | 4.5% Operation Plot | | | |
|---|---|---|---|---|---|---|---|---|---|
| | | Time Elapsed after Ingestion | | | | | | | |
| | | 30 min | 60 min | 90 min | 120 min | 30 min | 60 min | 90min | 120 min |
| Subject | 1 | 37.5 | 26.0 | 20.5 | 19.0 | 54.0 | 15.0 | 14.0 | 9.0 |
| No. | 2 | 47.0 | 17.0 | 15.0 | 22.0 | 33.5 | 14.0 | 10.0 | 14.5 |
| | 3 | 76.5 | 13.5 | 27.5 | 37.5 | 59.5 | 8.5 | 17.0 | 13.5 |
| | 4 | 34.0 | 28.5 | 3.5 | 5.5 | 26.0 | 25.0 | 16.5 | 8.5 |
| | 5 | 35.0 | 17.5 | 9.5 | 17.0 | 25.0 | 2.0 | 1.0 | 13.5 |

| | | | | | | | | |
|---|---|---|---|---|---|---|---|---|
| Average of | 46.0 | 20.5 | 15.2 | 20.2 | 39.6 | 12.9 | 11.7 | 11.8 |
| Subjects | | | | | | | | |

| | | | | |
|---|---|---|---|---|
| T-test against Control Plot | 34% | 4% | 45% | 13% |
| (Probability according to t-distribution) | | | | |

TABLE 31

unit: mg/dl

| Δ Cmax | | Control Plot | 4.5% Operation Plot |
|---|---|---|---|
| Subject No. | 1 | 37.5 | 54.0 |
| | 2 | 47.0 | 33.5 |
| | 3 | 76.5 | 59.5 |
| | 4 | 34.0 | 26.0 |
| | 5 | 35.0 | 25.0 |

| | | |
|---|---|---|
| Average of Subjects | 46.0 | 39.6 |
| Standard Deviation | 17.8 | 16.1 |

| | |
|---|---|
| T-test against Control Plot | 34.1% |
| (Probability according to t-distribution) | |

TABLE 32

unit: mg · min/dl

| Δ AUC | | Control Plot | 4.5% Operation Plot |
|---|---|---|---|
| Subject No. | 1 | 2805 | 2625 |
| | 2 | 2700 | 1943 |
| | 3 | 4088 | 2753 |
| | 4 | 2063 | 2153 |
| | 5 | 2115 | 1043 |

| | | |
|---|---|---|
| Average of Subjects | 2754 | 2103 |
| Standard Deviation | 817 | 680 |

| | |
|---|---|
| T-test against Control Plot | 7.1% |
| (Probability according to t-distribution) | |

[Measurement Results of Blood Glucose Level]

An average of the preprandial blood glucose levels of the 5 subjects was 86.4 to 89.5 mg/dl, and ΔCₘₐₓ was 46.0 mg/dl in the control plot, but was a low value of 39.6 mg/dl in the 4.5% calcium alginate operation plot. Besides, ΔAUC was 2754 mg·min/dl in the control plot, but was an obviously low value of 2103 mg·min/dl in the 4.5% calcium alginate operation plot.

In other words, it was confirmed, in boiled Japanese buckwheat noodles, that when 4.5% by mass of 270-mesh pass calcium alginate is added to a powder raw material for noodle production, the effect of suppressing the blood glucose level increase can be obtained.

### Example 7

<Sensory Evaluation Test for Checking Upper Limit of Addition of 270-Mesh Pass Calcium Alginate in Boiled Udon>: By using, as a control, the operation plot of 5% addition in which the effect of suppressing the blood glucose level increase and excellent texture was able to be obviously both obtained, 7%, 9% and 11% addition was performed respectively as a 5% operation plot, a 7% operation plot, a 9% comparative plot and an 11% comparative plot to check a range in which excellent texture can be obtained.

[Preparation Method for Samples]

Powder raw materials shown in [Table 33] were respectively homogenously mixed, and then, boiled udon samples of the respective operation plots and comparative plots were prepared in accordance with the preparation method for samples described in Example 1.

TABLE 33

| | Sample Type | | | | | | | |
|---|---|---|---|---|---|---|---|---|
| | 5% Opera- | | 7% Opera- | | 9% Compar- | | 11% Compar- | |
| Item | tion Plot | | tion Plot | | ative Plot | | ative Plot | |
| (Powder Raw | | | | | | | | |
| Materials) | | | | | | | | |
| Wheat Flour | 2760 | g | 2760 | g | 2760 | g | 2760 | g |
| mainly composed | | | | | | | | |
| of Kitahonami | | | | | | | | |
| Acetylated | 240 | g | 240 | g | 240 | g | 240 | g |
| Tapioca Starch | | | | | | | | |
| 270 Mesh Pass | 150 | g | 210 | g | 270 | g | 330 | g |
| Ca Alginate | | | | | | | | |
| Total of Powders | 3150 | g | 3210 | g | 3270 | g | 3330 | g |
| (Kneading Water) | | | | | | | | |
| Salt | 150.6 | g | 150.6 | g | 150.6 | g | 150.6 | g |
| Fresh Water | 1356 | g | 1356 | g | 1416 | g | 1386 | g |

[Sensory Evaluation Test]

As an evaluation method, a 10-point rating scale assuming that boiled udon of the control had a score of 5 was employed, and the 4 items of hardness (hard: 10 to soft: 0), chewy texture (chewy: 10 to brittle: 0), powderiness (powdery: 10 to not powdery: 0), and comprehensive texture (preferable: 10 to not preferable: 0) were scored with an integer, and an average of scores of 8 specialized panelists was employed.

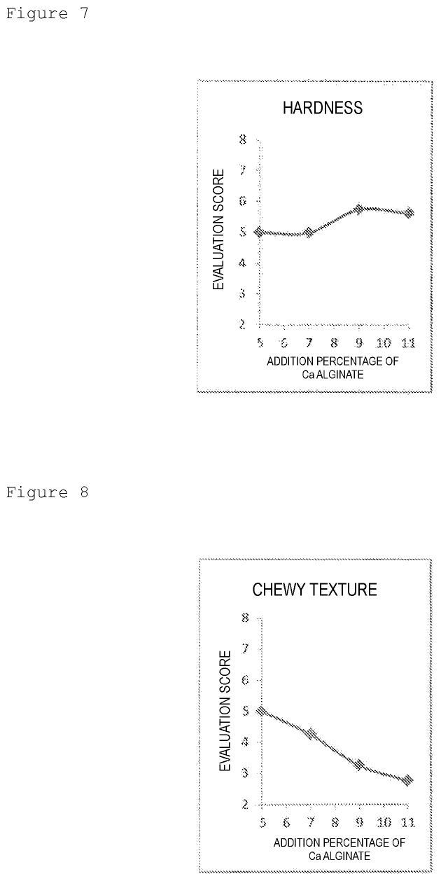

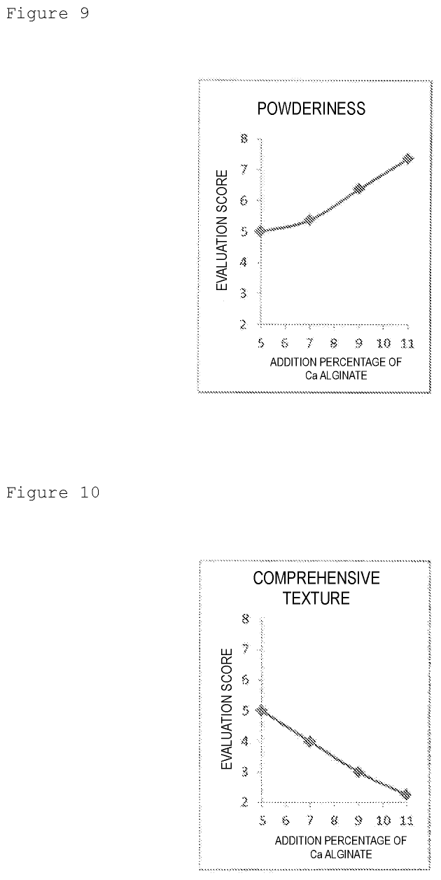

Results of the sensory evaluation test are shown in [Table 34], and graphs of evaluation scores obtained at the respective addition percentages of Ca alginate are illustrated in FIG. 7 (hardness), FIG. 8 (chewy texture), FIG. 9 (powderiness), and FIG. 10 (comprehensive texture).

TABLE 34
Boiled Udon different in Amount of Ca-Alg Added (5% as reference, 7%, 9%, 11%)

| | | Hardness | | | | Viscoelasticity (Chewy Texture) | | | |
|---|---|---|---|---|---|---|---|---|---|
| | | 5% | 7% | 9% | 11% | 5% | 7% | 9% | 11% |
| | | Operation | Operation | Comparative | Comparative | Operation | Operation | Comparative | Comparative |
| | | Plot | Plot | Plot | Plot | Plot | Plot | Plot | Plot |
| Evaluator | 1 | 5 (reference) | 6 | 8 | 7 | 5 (reference) | 5 | 4 | 5 |
| No. | 2 | 5 (reference) | 5 | 6 | 7 | 5 (reference) | 4 | 3 | 2 |
| | 3 | 5 (reference) | 6 | 6 | 8 | 5 (reference) | 3 | 4 | 2 |
| | 4 | 5 (reference) | 5 | 7 | 7 | 5 (reference) | 4 | 3 | 2 |
| | 5 | 5 (reference) | 4 | 3 | 2 | 5 (reference) | 5 | 3 | 2 |
| | 6 | 5 (reference) | 4 | 3 | 4 | 5 (reference) | 4 | 2 | 4 |
| | 7 | 5 (reference) | 6 | 6 | 6 | 5 (reference) | 5 | 4 | 3 |
| | 8 | 5 (reference) | 4 | 7 | 4 | 5 (reference) | 4 | 3 | 2 |
| Average | | | 5.0 | 5.8 | 5.6 | | 4.3 | 3.3 | 2.8 |
| | | Powderiness | | | | Comprehensive Texture | | | |
| | | 5% | 7% | 9% | 11% | 5% | 7% | 9% | 11% |
| | | Operation | Operation | Comparative | Comparative | Operation | Operation | Comparative | Comparative |
| | | Plot | Plot | Plot | Plot | Plot | Plot | Plot | Plot |
| Evaluator | 1 | 5 (reference) | 6 | 8 | 7 | 5 (reference) | 5 | 3 | 4 |
| No. | 2 | 5 (reference) | 5 | 6 | 7 | 5 (reference) | 4 | 3 | 2 |
| | 3 | 5 (reference) | 7 | 6 | 8 | 5 (reference) | 4 | 4 | 2 |
| | 4 | 5 (reference) | 5 | 7 | 6 | 5 (reference) | 4 | 3 | 2 |
| | 5 | 5 (reference) | 6 | 8 | 9 | 5 (reference) | 4 | 3 | 2 |
| | 6 | 5 (reference) | 5 | 4 | 5 | 5 (reference) | 4 | 3 | 4 |
| | 7 | 5 (reference) | 4 | 6 | 9 | 5 (reference) | 5 | 2 | 1 |
| | 8 | 5 (reference) | 5 | 6 | 8 | 5 (reference) | 2 | 3 | 1 |

| | | | | | | |
|---|---|---|---|---|---|---|
| Average | 5.4 | 6.4 | 7.4 | 4.0 | 3.0 | 2.3 |

As a result, as an average score of the “item of hardness,” as compared with the 5% operation plot of the control (reference score of 5), the 7% operation plot had a score of 5.0, the 9% comparative plot had a score of 5.8 and the 11% comparative plot had a score of 5.6, and thus, there was not a difference of a score of 1 in all the operation plot and comparative plots as compared with the reference score of 5. As an average score of the “item of chewy texture”, as compared with the 5% operation plot of the control (reference score of 5), the 7% operation plot had a score of 4.3, the 9% comparative plot had a score of 3.3 and the 11% comparative plot had a score of 2.8, and thus, there was not a difference of a score of 1 in the 7% operation plot as compared with the reference score of 5, and was a difference of a score of 1 or more in all the comparative plots as compared with the reference score of 5. As an average score of the “item of powderiness,” as compared with the 5% operation plot of the control (reference score of 5), the 7% operation plot had a score of 5.4, the 9% comparative plot had a score of 6.4 and the 11% comparative plot had a score of 7.4, and thus, there was not a difference of a score of 1 in the 7% operation plot as compared with the reference score of 5, and was a difference of a score of 1 or more in all the comparative plots as compared with the reference score of 5. As an average score of the “item of comprehensive texture,” as compared with the 5% operation plot of the control (reference score of 5), the 7% operation plot had a score of 4, the 9% comparative plot had a score of 3 and the 11% comparative plot had a score of 2.3, and thus, there was a difference of a score of just 1 in the 7% operation plot as compared with the reference score of 5, and was a difference of a score of 2 or more in all the comparative plots as compared with the reference score of 5.

That there was not a difference of a score of 1 in the average score of the 8 panelists from the reference indicates that there was a panelist that determined there was no difference, and hence can be a rough standard indicating the range of equivalence. Therefore, the 5% operation plot of the control and the 7% operation plot were equivalent in the three items of the hardness, the chewy texture and the powderiness, and it is revealed that they were within a range where excellent texture can be obtained. Besides, in the graphs of the items of the chewy texture and the powderiness, an inclination between the 7% operation plot and the 9% comparative plot is larger than an inclination between the 5% operation plot and the 7% operation plot, and an inclination between the 9% comparative plot and the 11% comparative plot. This indicates that the chewy texture is rapidly deteriorated and the powderiness is rapidly increased between the addition percentages of 7% and 9%. These indicate critical significance of defining an addition percentage of 8% as the upper limit value.

### Example 8

<Chilled Boiled Buckwheat Noodles>: The effect of suppressing the postprandial blood glucose level increase of boiled buckwheat noodles in which Ca alginate was added was checked, and sensory evaluation was performed to compare 270 mesh pass and 80 mesh pass.

[Preparation of Boiled Buckwheat Noodle Samples]

Powder raw materials shown in [Table 35] were respectively homogeneously mixed, and kneading water was respectively added thereto, followed by mixing. As a powder raw material, a wheat flour and a buckwheat flour (crude protein: 22.4%, ash: 3.0%) mainly ground from an outer layer were mixed with modified starch and vital gluten, so as to improve texture and enforce binding of a dough. Ca alginate was added in a comparative plot and an operation plot, and glycine was dissolved in the kneading water for purposes of improving the preserving property. The mixing was performed by kneading at a decompression degree of 80 kPa for 8 minutes to obtain a kneaded dough in a crumbled state at a temperature of 28 to 34° C.

The kneaded dough was formed into crude noodle belts each having a thickness of 6 mm using a noodle belt forming roll to be compounded with the thickness kept, the resultant was allowed to stand at 25° C. for 60 minutes under a condition where it was not dried, was rolled into a thickness of 1.50 mm using a four-stage flat roll, and then finely cut into noodle strings using mixed cutting teeth having grove widths of 2.1 mm:1.7 mm:1.5 mm=occupied widths of 12:19:21. The thus obtained noodle strings were boiled in hot water for boiling at 98° C. for 1 minute and 10 seconds, then somewhat cooled by putting them in water at 15° C. twice, and dipped in a 0.4% by mass acetic acid solution at 5° C. for 2 minutes and 40 seconds.

Thereafter, the solution remaining thereon was rapidly removed, 160 g thereof corresponding to each serving was sealed with a polyethylene film, subjected to steam sterilization at 85° C. for 30 minutes, and rapidly cooled in a refrigerator at 10° C. to prepare chilled boiled buckwheat noodles having a boiled noodle moisture shown in [Table 35].

TABLE 35

| | Sample Type | | | | | |
|---|---|---|---|---|---|---|
| | | | 80 Mesh | | 270 Mesh | |
| | | | Comparative | | Operation | |
| Item | Control | | Plot | | Plot | |
| (Powder Raw Materials) | | | | | | |
| Strong Wheat Flour | 1932 | g | 1776 | g | 1776 | g |
| Outer Layer Buckwheat Flour | 966 | g | 966 | g | 966 | g |
| Modified Starch for | 96 | g | 96 | g | 96 | g |
| Buckwheat Noodles | | | | | | |
| Vital Gluten | 126 | g | 126 | g | 126 | g |
| 80 Mesh Pass Ca Alginate | | | 156 | g | | |
| 270 Mesh Pass Ca Alginate | | | | | 156 | g |
| Total of Powders | 3120 | g | 3120 | g | 3120 | g |
| (Kneading Water) | | | | | | |
| Glycine | 35.4 | g | 35.4 | g | 35.4 | g |
| Fresh Water | 996 | g | 1056 | g | 1056 | g |

| | | | |
|---|---|---|---|
| Boiling Time | 1 min 10 sec | 1 min 10 sec | 1 min 10 sec |
| * Boiled Noodle Average | 63.0% | 65.0% | 65.0% |
| Moisture | | | |

[Sensory Evaluation Test]

For cooking, each sample was boiled in boiling water for 1 minute. Sensory evaluation was performed on a 80 mesh comparative plot and a 270 mesh operation plot by using a control plot as a reference. By employing a 5-point rating scale assuming that boiled buckwheat noodles of the control plot had a score of 3, the four items of hardness (hard: 5 to soft: 1), viscoelasticity (viscoelastic: 5 to brittle: 1), powderiness (powdery: 5 to not powdery: 1), and comprehensive texture (preferable: 5 to not preferable: 1) were evaluated, and an average of scores of 5 specialized panelists was employed.

TABLE 36

| | | Hardness | | | Viscoelasticity | | | |
|---|---|---|---|---|---|---|---|---|
| | | | 80 Mesh | 270 Mesh | | 80 Mesh | 270 Mesh | |
| | | | Comparative | Operation | | Comparative | Operation | Powderiness |
| | | Control | Plot | Plot | Control | Plot | Plot | Control |
| Evaluator | 1 | 3 (reference) | 3.0 | 3.0 | 3 (reference) | 3.0 | 3.0 | 3 (reference) |
| No. | 2 | 3 (reference) | 2.0 | 2.5 | 3 (reference) | 2.0 | 2.5 | 3 (reference) |
| | 3 | 3 (reference) | 2.0 | 2.5 | 3 (reference) | 2.5 | 3.0 | 3 (reference) |
| | 4 | 3 (reference) | 2.0 | 3.0 | 3 (reference) | 2.0 | 2.5 | 3 (reference) |
| | 5 | 3 (reference) | 4.0 | 4.0 | 3 (reference) | 4.0 | 3.0 | 3 (reference) |

| | | | | |
|---|---|---|---|---|
| Average | 2.6 | 3.0 | 2.7 | 2.8 |
| Standard | 0.89 | 0.61 | 0.84 | 0.27 |
| Deviation | | | | |

| | | |
|---|---|---|
| T-test against | 9.9% | 74.9% |
| Comparative Plot | | |
| (Probability | | |
| according to t- | | |
| distribution) | | |

| | | | Powderiness | | Comprehensive Texture | | |
|---|---|---|---|---|---|---|---|
| | | | 80 Mesh | 270 Mesh | | 80 Mesh | 270 Mesh |
| | | Hardness | Comparative | Operation | | Comparative | Operation |
| | | Control | Plot | Plot | Control | Plot | Plot |
| Evaluator | 1 | 3 (reference) | 3.5 | 3.5 | 3 (reference) | 3.0 | 3.0 |
| No. | 2 | 3 (reference) | 4.0 | 3.5 | 3 (reference) | 2.0 | 2.5 |
| | 3 | 3 (reference) | 4.0 | 3.0 | 3 (reference) | 2.5 | 2.5 |
| | 4 | 3 (reference) | 4.0 | 3.5 | 3 (reference) | 2.0 | 2.5 |
| | 5 | 3 (reference) | 2.0 | 2.0 | 3 (reference) | 3.5 | 3.5 |

| | | | | |
|---|---|---|---|---|
| Average | 3.5 | 3.1 | 2.6 | 2.8 |
| Standard | 0.87 | 0.65 | 0.65 | 0.45 |
| Deviation | | | | |

| | | |
|---|---|---|
| T-test against | 9.9% | 17.8% |
| Comparative Plot | | |
| (Probability | | |
| according to t- | | |
| distribution) | | |

Results are shown in [Table 36]. As a result, in the item of hardness, as compared with the reference score of 3 of the control plot, the 270 mesh operation plot had the same score and the 80 mesh comparative plot had a score of a low value of 2.6. In the item of viscoelasticity, as compared with the reference score of 3 of the control plot, there was a tendency of successively lowering from a score of 2.8 in the 270 mesh operation plot to a score of 2.7 in the 80 mesh comparative plot. In the item of powderiness, as compared with the reference score of 3 of the control plot, there was a tendency of successively increasing from a score of 3.1 in the 270 mesh operation plot to a score of 3.5 in the 80 mesh comparative plot. In the item of comprehensive texture, as compared with the reference score of 3 of the control plot, there was a tendency of successively lowering from a score of 2.8 in the 270 mesh operation plot to a score of 2.6 in the 80 mesh comparative plot. Based on these results, the 270 mesh operation plot had the scores closer to those of the control plot in all the items than the 80 mesh comparative plot.

[Measurement Method for Blood Glucose Level]

For the measurement of the blood glucose level, comparison was made between the 80 mesh comparative plot and the 270 mesh operation plot with the control plot excluded. The measurement of the blood glucose level was performed in Shimadaya Corporation, and subjects were 5 healthy males and females over 20 years old including a person having a fasting blood glucose level corresponding to borderline diabetes. For cooking, 160 g corresponding to each serving of each sample was boiled for 1 minute in boiling water to be served as kake soba with no ingredients. Conditions were adjusted so that the buckwheat noodles were able to be finished in 5 minutes after serving, and the blood glucose level was measured 30 minutes, 60 minutes, 90 minutes and 120 minutes after starting the ingestion.

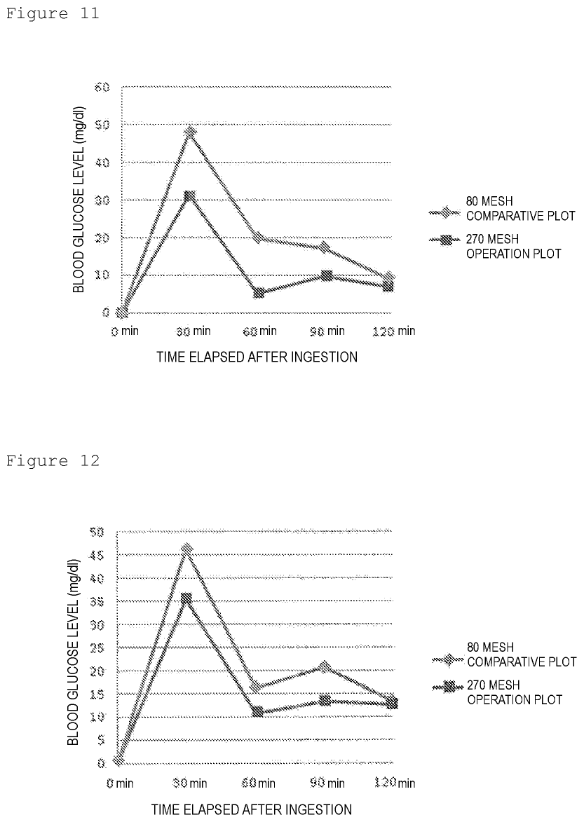

The thus obtained blood glucose level is shown in [Table 37], a change in the blood glucose level is shown in [Table 38], a difference (ΔCₘₐₓ) between a maximum blood glucose level and preprandial blood glucose level is shown in [Table 39], and a product (ΔAUC) of the blood glucose level and the time calculated using the preprandial blood glucose level as a baseline is shown in [Table 40]. Besides, transition of an average of the blood glucose levels is illustrated in a graph of FIG. 11.

TABLE 37
unit: mg/dl

| | | 80 Mesh Comparative Plot | | | | | 270 Mesh Operation Plot | | | | |
|---|---|---|---|---|---|---|---|---|---|---|---|
| | | Time Elapsed after Ingestion | | | | | | | | | |
| | | Prior | 30 min | 60 min | 90 min | 120 min | Prior | 30 min | 60 min | 90 min | 120 min |
| Subject | 1 | 89.5 | 149.0 | 146,0 | 131.0 | 103.5 | 94.0 | 123.0 | 109.0 | 110.0 | 106.0 |
| No. | 2 | 97.0 | 142.5 | 98.5 | 106.0 | 106.0 | 107.5 | 144.5 | 100.5 | 110.5 | 117.5 |
| | 3 | 95.5 | 124.0 | 101.5 | 100.5 | 102.5 | 90.0 | 113.0 | 92.5 | 105.0 | 93.0 |
| | 4 | 89.5 | 141.5 | 113.5 | 97.0 | 93.5 | 88.5 | 114.0 | 97.5 | 96.5 | 90.5 |
| | 5 | 88.0 | 143.0 | 98.5 | 110.0 | 98.5 | 89.0 | 131.0 | 95.0 | 95.5 | 96.5 |

| | | | | | | | | | | |
|---|---|---|---|---|---|---|---|---|---|---|
| Average of | 91.9 | 140.0 | 111.6 | 10B.9 | 100.8 | 93.8 | 125.1 | 98.9 | 103.5 | 100.7 |
| Subjects | | | | | | | | | | |

| | | | | | |
|---|---|---|---|---|---|
| T-test against 80 mesh Operation Plot | 52% | 5% | 13% | 36% | 98% |
| (Probability according to t-distribution) | | | | | |

TABLE 38
unit: mg/dl

| | | 80 Mesh Comparative Plot | | | | 270 Mesh Operation Plot | | | |
|---|---|---|---|---|---|---|---|---|---|
| | | Time Elapsed after Ingestion | | | | | | | |
| | | 30 min | 60 min | 90 min | 120 min | 30 min | 60 min | 90 min | 120 min |
| Subject | 1 | 59.5 | 56.5 | 41.5 | 14.0 | 29.0 | 15.0 | 16.0 | 12.0 |
| No. | 2 | 45.5 | 1.5 | 9.0 | 9.0 | 37.0 | −7.0 | 3.0 | 10.0 |
| | 3 | 28.5 | 6.0 | 5.0 | 7.0 | 23.0 | 2.5 | 15.0 | 3.0 |
| | 4 | 52.0 | 24.0 | 7.5 | 4.0 | 25.5 | 9.0 | 8.0 | 2.0 |
| | 5 | 55.0 | 10.5 | 22.0 | 10.5 | 42.0 | 6.0 | 6.5 | 7.5 |

| | | | | | | | | |
|---|---|---|---|---|---|---|---|---|
| Average of | 48.1 | 19.7 | 17.0 | 8.9 | 31.3 | 5.1 | 9.7 | 6.9 |
| Subjects | | | | | | | | |

| | | | | |
|---|---|---|---|---|
| T-test against 80 Mesh Operation Plot | 3% | 11% | 30% | 8% |
| (Probability according to t-distribution) | | | | |

TABLE 39
unit: mg/dl

| | 80 Mesh | 270 Mesh |
|---|---|---|
| Δ Cmax | Comparative Plot | Operation Plot |

| | | | |
|---|---|---|---|
| Subject No. | 1 | 59.5 | 29.0 |
| | 2 | 45.5 | 37.0 |
| | 3 | 28.5 | 23.0 |
| | 4 | 52.0 | 25.5 |
| | 5 | 55.0 | 42.0 |

| | | |
|---|---|---|
| Average of Subjects | 48.1 | 31.3 |
| Standard Deviation | 12.1 | 8.0 |

| | |
|---|---|
| T-test against 80 Mesh Comparative Plot | 2.8% |
| (Probability according to t-distribution) | |

TABLE 40
unit: mg · min/dl

| | 80 Mesh | 270 Mesh |
|---|---|---|
| Δ AUC | Comparative Plot | Operation Plot |

| | | | |
|---|---|---|---|
| Subject No. | 1 | 4935 | 1980 |
| | 2 | 1815 | 1140 |
| | 3 | 1290 | 1260 |
| | 4 | 2565 | 1305 |
| | 5 | 2783 | 1748 |

| | | |
|---|---|---|
| Average of Subjects | 2678 | 1487 |
| Standard Deviation | 1395 | 359 |

| | |
|---|---|
| T-test against 80 Mesh Comparative Plot | 7.1% |
| (Probability according to t-distribution) | |

[Measurement Results of Blood Glucose Level]

An average of the preprandial blood glucose levels of the 5 subjects was 91.9 to 93.8 mg/dl, ΔCₘₐₓ was 48.1 mg/dl in the 80 mesh comparative plot, but was an obviously low value of 31.3 mg/dl in the 270 mesh operation plot, and a significant difference was found by a t-test. Besides, ΔAUC was 2678 mg·min/dl in the 80 mesh comparative plot, but was an obviously low value of 1487 mg·min/dl in the 270 mesh operation plot.

In other words, it was confirmed that the effect of suppressing the blood glucose level increase can be obtained also in Japanese boiled buckwheat noodles by using calcium alginate pulverized to 270 mesh pass as compared with that of 80 mesh pass.

### Example 9

<Chilled Boiled Chinese Noodles>: The effect of suppressing the postprandial blood glucose level increase of boiled Chinese noodles in which Ca alginate was added was checked, and sensory evaluation was performed to compare 270 mesh pass and 80 mesh pass.

[Preparation of Boiled Chinese Noodle Samples]

Powder raw materials shown in [Table 41] were respectively homogeneously mixed, and kneading water was respectively added thereto, followed by mixing. As a powder raw material, a wheat flour for Chinese noodles was mixed with vital gluten and an egg white powder, so as to enforce binding of a dough and improve texture. Ca alginate was added in a comparative plot and an operation plot, and kansui (an alkaline solution or powder used for Chinese noodles) and a pigment were dissolved in the kneading water in addition to glycine used for purposes of improving the preserving property. The mixing was performed by kneading at a decompression degree of 80 kPa for 11 minutes to obtain a kneaded dough in a crumbled state at a temperature of 28 to 34° C. The kneaded dough was formed into crude noodle belts each having a thickness of 10 mm using a noodle belt forming roll to be compounded with the thickness kept, the resultant was allowed to stand at 25° C. for 60 minutes under a condition where it was not dried, was rolled into a thickness of 2.45 mm using a four-stage flat roll, and then finely cut into noodle strings using cutting teeth having a grove width of 1.9 mm.

The thus obtained noodle strings were boiled in hot water for boiling at 98° C. for 1 minute and 45 seconds, then somewhat cooled by putting them in water at 15° C. twice, and then cooled with ice water at 5° C. for 1 minute and 20 seconds. Thereafter, the solution remaining thereon was rapidly removed, 150 g thereof corresponding to each serving was sealed with a polyethylene film, subjected to steam sterilization at 85° C. for 30 minutes, and rapidly cooled in a refrigerator at 10° C. to prepare chilled boiled Chinese noodles having a boiled noodle moisture shown in [Table 41].

TABLE 41

| | Sample Type | | | | | |
|---|---|---|---|---|---|---|
| | | | 80 Mesh | | 270 Mesh | |
| | | | Comparative | | Operation | |
| Item | Control | | Plot | | Plot | |
| (Powder Raw Materials) | | | | | | |
| Wheat Flour | 3000 | g | 3000 | g | 3000 | g |
| Vital Gluten | 90 | g | 90 | g | 90 | g |
| Egg White Powder | 30 | g | 30 | g | 30 | g |
| 80 Mesh Pass Ca Alginate | | | 156 | g | | |
| 270 Mesh Pass Ca Alginate | | | | | 156 | g |
| Total of Powders | 3120 | g | 3276 | g | 3276 | g |
| (Kneading Water) | | | | | | |
| Glycine | 36 | g | 36 | g | 36 | g |
| Salt | 30 | g | 30 | g | 30 | g |
| Kansui (Powder) | 27 | g | 27 | g | 27 | g |
| Gardenia Pigment | 6 | g | 6 | g | 6 | g |
| Fresh Water | 993 | g | 1260 | g | 1260 | g |

| | | | |
|---|---|---|---|
| Boiling Time | 1 min 45 sec | 1 min 45 sec | 1 min 45 sec |
| * Boiled Noodle Moisture | 65.0% | 68.0% | 68.0% |

[Sensory Evaluation Test]

For cooking, each sample was boiled in boiling water for 1 minute. Sensory evaluation was performed on a 80 mesh comparative plot and a 270 mesh operation plot by using a control plot as a reference. By employing a 5-point rating scale assuming that boiled Chinese noodles of the control plot had a score of 3, the four items of hardness (hard: 5 to soft: 1), viscoelasticity (viscoelastic: 5 to brittle: 1), powderiness (powdery: 5 to not powdery: 1), and comprehensive texture (preferable: 5 to not preferable: 1) were evaluated, and an average of scores of 5 specialized panelists was employed.

TABLE 42

| | | Hardness | | | Viscoelasticity | | | |
|---|---|---|---|---|---|---|---|---|
| | | | 80 Mesh | 270 Mesh | | 80 Mesh | 270 Mesh | |
| | | | Comparative | Operation | | Comparative | Operation | Powderiness |
| | | Control | Plot | Plot | Control | Plot | Plot | Control |
| Evaluator | 1 | 3 (reference) | 2.5 | 2.5 | 3 (reference) | 3.5 | 3.5 | 3 (reference) |
| No. | 2 | 3 (reference) | 2.0 | 2.5 | 3 (reference) | 1.5 | 2.0 | 3 (reference) |
| | 3 | 3 (reference) | 2.5 | 3.0 | 3 (reference) | 3.0 | 3.0 | 3 (reference) |
| | 4 | 3 (reference) | 3.0 | 3.0 | 3 (reference) | 2.0 | 3.0 | 3 (reference) |
| | 5 | 3 (reference) | 2.0 | 3.0 | 3 (reference) | 2.0 | 3.0 | 3 (reference) |

| | | | | |
|---|---|---|---|---|
| Average | 2.4 | 2.8 | 2.4 | 2.9 |
| Standard | 0.4 | 0.3 | 0.8 | 0.5 |
| Deviation | | | | |

| | | |
|---|---|---|
| T-test against | 9.9% | 8.9% |
| Comparative Plot | | |
| (Probability | | |
| according to t- | | |
| distribution) | | |

| | | | Powderiness | | Comprehensive Texture | | |
|---|---|---|---|---|---|---|---|
| | | | 80 Mesh | 270 Mesh | | 80 Mesh | 270 Mesh |
| | | Hardness | Comparative | Operation | | Comparative | Operation |
| | | Control | Plot | Plot | Control | Plot | Plot |
| Evaluator | 1 | 3 (reference) | 3.5 | 3.0 | 3 (reference) | 3.0 | 3.0 |
| No. | 2 | 3 (reference) | 4.0 | 3.5 | 3 (reference) | 2.0 | 2.5 |
| | 3 | 3 (reference) | 2.5 | 2.5 | 3 (reference) | 3.0 | 3.0 |
| | 4 | 3 (reference) | 2.5 | 3.0 | 3 (reference) | 2.0 | 2.5 |
| | 5 | 3 (reference) | 3.5 | 3.0 | 3 (reference) | 2.0 | 3.0 |

| | | | | |
|---|---|---|---|---|
| Average | 3.2 | 3.0 | 2.4 | 2.8 |
| Standard | 0.7 | 0.4 | 0.5 | 0.3 |
| Deviation | | | | |

| | | |
|---|---|---|
| T-test against | 37.4% | 9.9% |
| Comparative Plot | | |
| (Probability | | |
| according to t- | | |
| distribution) | | |

Results are shown in [Table 42]. As a result, in the item of hardness, as compared with the reference score of 3 of the control plot, there was a tendency of successively lowering from a score of 2.8 in the 270 mesh operation plot to a score of 2.4 in the 80 mesh comparative plot. In the item of viscoelasticity, as compared with the reference score of 3 of the control plot, there was a tendency of successively lowering from a score of 2.9 in the 270 mesh operation plot to a score of 2.4 in the 80 mesh comparative plot. In the item of powderiness, as compared with the reference score of 3 of the control plot, the 270 mesh operation plot had the same score and the 80 mesh comparative plot had a score of a high value of 3.2. In the item of comprehensive texture, as compared with the reference score of 3 of the control plot, there was a tendency of successively lowering from a score of 2.8 in the 270 mesh operation plot to a score of 2.4 in the 80 mesh comparative plot.

Based on these results, the 270 mesh operation plot had the scores closer to those of the control plot in all the items than the 80 mesh comparative plot.

[Measurement Method for Blood Glucose Level]

For the measurement of the blood glucose level, comparison was made between the 80 mesh comparative plot and the 270 mesh operation plot with the control plot excluded. The measurement of the blood glucose level was performed in Shimadaya Corporation, and subjects were 5 healthy males and females over 20 years old including a person having a fasting blood glucose level corresponding to borderline diabetes. For cooking, 150 g corresponding to each serving of each sample was boiled for 1 minute in boiling water to be served as ramen in a soy-sauce flavored soup with no ingredients. Conditions were adjusted so that the ramen was able to be finished in 5 minutes after serving, and the blood glucose level was measured 30 minutes, 60 minutes, 90 minutes and 120 minutes after starting the ingestion.

The thus obtained blood glucose level is shown in [Table 43], a change in the blood glucose level is shown in [Table 44], a difference (ΔCₘₐₓ) between a maximum blood glucose level and preprandial blood glucose level is shown in [Table 45], and a product (ΔAUC) of the blood glucose level and the time calculated using the preprandial blood glucose level as a baseline is shown in [Table 46]. Besides, transition of an average of the blood glucose levels is illustrated in a graph of FIG. 12.

TABLE 43
Unit: mg/dl

| | | 80 Mesh Comparative Plot | | | | | 270 Mesh Operation Plot | | | | |
|---|---|---|---|---|---|---|---|---|---|---|---|
| | | Time Elapsed after Ingestion | | | | | | | | | |
| | | Prior | 30 min | 60 min | 90 min | 120 min | Prior | 30 min | 60 min | 90 min | 120 min |
| Subject | 1 | 99.0 | 154.0 | 137.0 | 133.5 | 121.5 | 98.0 | 145.0 | 123.0 | 111.5 | 115.5 |
| No. | 2 | 92.0 | 144.0 | 101.5 | 116.0 | 109.0 | 94.5 | 124.5 | 99.5 | 109.0 | 99.5 |
| | 3 | 98.0 | 126.5 | 101.5 | 115.0 | 108.0 | 95.5 | 116.5 | 106.0 | 113.5 | 107.0 |
| | 4 | 98.0 | 139.5 | 101.5 | 106.0 | 104.0 | 91.0 | 123.5 | 94.5 | 101.5 | 105.5 |
| | 5 | 99.0 | 153.0 | 125.0 | 119.0 | 110.5 | 94.0 | 141.5 | 103.5 | 104.5 | 108.5 |

| | | | | | | | | | | |
|---|---|---|---|---|---|---|---|---|---|---|
| Average of | 97.2 | 143.4 | 113.3 | 117.9 | 110.6 | 94.6 | 130.2 | 105.3 | 108.0 | 107.2 |
| Subjects | | | | | | | | | | |

| | | | | | |
|---|---|---|---|---|---|
| T-test against 80 Mesh Comparative Plot | 19% | 0.3% | 15% | 6% | 16% |
| (Probability according to t-distribution) | | | | | |

TABLE 44
unit: mg/dl

| | | 80 Mesh Comparative Plot | | | | 270 Mesh Operation Plot | | | |
|---|---|---|---|---|---|---|---|---|---|
| | | Time Elapsed after Ingestion | | | | | | | |
| | | 30 min | 60 min | 90 min | 120 min | 30 min | 60 min | 90 min | 120 min |
| Subject | 1 | 55.0 | 38.0 | 34.5 | 22.5 | 47.0 | 25.0 | 13.5 | 17.5 |
| No. | 2 | 52.0 | 9.5 | 24.0 | 17.0 | 30.0 | 5.0 | 14.5 | 5.0 |
| | 3 | 28.5 | 3.5 | 17.0 | 10.0 | 21.0 | 10.5 | 18.0 | 11.5 |
| | 4 | 41.5 | 3.5 | 8.0 | 6.0 | 32.5 | 3.5 | 10.5 | 14.5 |
| | 5 | 54.0 | 26.0 | 20.0 | 11.5 | 47.5 | 9.5 | 10.5 | 14.5 |

| | | | | | | | | |
|---|---|---|---|---|---|---|---|---|
| Average of | 46.2 | 16.1 | 20.7 | 13.4 | 35.6 | 10.7 | 13.4 | 12.6 |
| Subjects | | | | | | | | |

| | | | | |
|---|---|---|---|---|
| T-test against 80 Mesh Comparative Plot | 2% | 27% | 16% | 83% |
| (Probability according to t-distribution) | | | | |

TABLE 45
unit: mg/dl

| | 80 Mesh | 270 Mesh |
|---|---|---|
| Δ Cmax | Comparative Plot | Operation Plot |

| | | | |
|---|---|---|---|
| Subject No. | 1 | 55.0 | 47.0 |
| | 2 | 52.0 | 30.0 |
| | 3 | 28.5 | 21.0 |
| | 4 | 41.5 | 32.5 |
| | 5 | 54.0 | 47.5 |

| | | |
|---|---|---|
| Average of Subjects | 46.2 | 35.6 |
| Standard Deviation | 11.3 | 11.5 |

| | |
|---|---|
| T-test against 80 Mesh Comparative Plot | 2.1% |
| (Probability according to t-distribution) | |

TABLE 46
unit: mg · min/dl

| | 80 Mesh | 270 Mesh |
|---|---|---|
| Δ AUC | Comparative Plot | Operation Plot |

| | | | |
|---|---|---|---|
| Subject No. | 1 | 4163 | 2828 |
| | 2 | 2820 | 1560 |
| | 3 | 1620 | 1658 |
| | 4 | 1680 | 1613 |
| | 5 | 3173 | 2243 |

| | | |
|---|---|---|
| Average of Subjects | 2691 | 1980 |
| Standard Deviation | 1070 | 548 |

| | |
|---|---|
| T-test against 80 Mesh Comparative Plot | 7.2% |
| (Probability according to t-distribution) | |

[Measurement Results of Blood Glucose Level]

An average of the preprandial blood glucose levels of the 5 subjects was 94.6 to 97.2 mg/dl, ΔCₘₐₓ was 46.2 mg/dl in the 80 mesh comparative plot, but was an obviously low value of 35.6 mg/dl in the 270 mesh operation plot, and a significant difference was found by a t-test. Besides, ΔAUC was 2691 mg·min/dl in the 80 mesh comparative plot, but was an obviously low value of 1980 mg·min/dl in the 270 mesh operation plot.

In other words, it was confirmed that the effect of suppressing the blood glucose level increase can be obtained also in boiled Chinese noodles by using calcium alginate pulverized to 270 mesh pass as compared with that of 80 mesh pass.

### Example 10

<Raw Udon>: The effect of suppressing the postprandial blood glucose level increase of raw udon in which Ca alginate was added was checked, and sensory evaluation was performed to compare 270 mesh pass and 80 mesh pass.

[Preparation of Raw Udon Samples]

Powder raw materials shown in [Table 47] were respectively homogeneously mixed, and kneading water was respectively added thereto, followed by mixing. As a powder raw material, a wheat flour was mixed with acetylated tapioca starch, so as to improve texture. Ca alginate was added in a comparative plot and an operation plot, and alcohol and salt were dissolved in the kneading water for purposes of improving the preserving property and noodle making properties. The mixing was performed by kneading under normal pressure of a decompression degree of 80 kPa for 12 minutes to obtain a kneaded dough in a crumbled state at a temperature of 28 to 34° C.

The kneaded dough was formed into crude noodle belts each having a thickness of 10 mm using a noodle belt forming roll to be compounded with the thickness kept, the resultant was allowed to stand at 25° C. for 60 minutes under a condition where it was not dried, was rolled into a thickness of 2.15 mm using a four-stage flat roll, and then finely cut into noodle strings using mixed cutting teeth having grove widths of 1.9 mm:2.2 mm:2.5 mm=occupied widths of 33:27:19, and 100 g thereof corresponding to each serving was sealed with a polyethylene film after powder sprinkling.

TABLE 47

| | Sample Type | | | | | |
|---|---|---|---|---|---|---|
| | | | 80 Mesh | | 270 Mesh | |
| | | | Comparative | | Operation | |
| Item | Control | | Plot | | Plot | |
| (Powder Raw Materials) | | | | | | |
| Wheat Flour mainly composed of | 2813 | g | 2813 | g | 2813 | g |
| Kitahonami | | | | | | |
| Acetylated Tapioca Starch | 187 | g | 187 | g | 187 | g |
| 80 Mesh Pass Ca Alginate | | | 150 | g | | |
| 270 Mesh Pass Ca Alginate | | | | | 150 | g |
| Total of Powders | 3000 | g | 3150 | g | 3150 | g |
| (Kneading Water) | | | | | | |
| Alcohol | 150 | g | 150 | g | 150 | g |
| Salt | 84.9 | g | 113.8 | g | 113.8 | |
| Fresh Water | 795 | g | 1024 | g | 1024 | g |

[Sensory Evaluation Test]

For cooking, each sample was boiled in boiling water for 5 minutes and 30 seconds. Sensory evaluation was performed on a 80 mesh comparative plot and a 270 mesh operation plot by using a control plot as a reference. By employing a 5-point rating scale assuming that the control plot had a score of 3, the four items of hardness (hard: 5 to soft: 1), viscoelasticity (viscoelastic: 5 to brittle: 1), powderiness (powdery: 5 to not powdery: 1), and comprehensive texture (preferable: 5 to not preferable: 1) were evaluated, and an average of scores of 4 specialized panelists was employed.

TABLE 48

| | | Hardness | | | Viscoelasticity | | |
|---|---|---|---|---|---|---|---|
| | | | 80 Mesh | 270 Mesh | | 80 Mesh | 270 Mesh |
| | | | Comparative | Operation | | Comparative | Operation |
| | | Control | Plot | Plot | Control | Plot | Plot |
| Evaluator No. | 1 | 3 | 3.5 | 3.0 | 3 | 2.0 | 2.5 |
| | | (reference) | | | (reference) | | |
| | 2 | 3 | 2.0 | 3.0 | 3 | 2.0 | 2.5 |
| | | (reference) | | | (reference) | | |
| | 3 | 3 | 2.0 | 3.0 | 3 | 2.5 | 3.0 |
| | | (reference) | | | (reference) | | |
| | 4 | 3 | 2.0 | 3.0 | 3 | 2.0 | 2.5 |
| | | (reference) | | | (reference) | | |
| Average | | | 2.4 | 2.8 | | 2.1 | 2.6 |
| Standard | | | 0.8 | 0.5 | | 0.3 | 0.3 |
| Deviation | | | | | | | |
| T-test against | | | | 39.1% | | | 5%> |
| Comparative Plot | | | | | | | |
| (Probability | | | | | | | |
| according to t- | | | | | | | |
| distribution) | | | | | | | |
| | | Powderiness | | | Comprehensive Texture | | |
| | | | 80 Mesh | 270 Mesh | | 80 Mesh | 270 Mesh |
| | | | Comparative | Operation | | Comparative | Operation |
| | | Control | Plot | Plot | Control | Plot | Plot |
| Evaluator No. | 1 | 3 | 4.5 | 3.5 | 3 | 2.0 | 3.0 |
| | | (reference) | | | (reference) | | |
| | 2 | 3 | 3.0 | 3.0 | 3 | 2.0 | 2.5 |
| | | (reference) | | | (reference) | | |
| | 3 | 3 | 2.5 | 3.0 | 3 | 2.5 | 3.0 |
| | | (reference) | | | (reference) | | |
| | 4 | 3 | 4.0 | 3.0 | 3 | 2.0 | 2.5 |
| | | (reference) | | | (reference) | | |

| | | | | |
|---|---|---|---|---|
| Average | 3.5 | 3.1 | 2.1 | 2.8 |
| Standard | 0.9 | 0.3 | 0.3 | 0.3 |
| Deviation | | | | |

| | | |
|---|---|---|
| T-test against | 39.1% | 1.5% |
| Comparative Plot | | |
| (Probability | | |
| according to t- | | |
| distribution) | | |

Results are shown in [Table 48]. As a result, in the item of hardness, as compared with the reference score of 3 of the control plot, there was a tendency of successively lowering from a score of 2.8 in the 270 mesh operation plot to a score of 2.4 in the 80 mesh comparative plot. In the item of viscoelasticity, as compared with the reference score of 3 of the control plot, there was a tendency of successively lowering from a score of 2.6 in the 270 mesh operation plot to a score of 2.1 in the 80 mesh comparative plot. In the item of powderiness, as compared with the reference score of 3 of the control plot, there was a tendency of successively increasing from a score of 3.1 in the 270 mesh operation plot to a score of 3.5 in the 80 mesh comparative plot. In the item of comprehensive texture, as compared with the reference score of 3 of the control plot, there was a tendency of successively lowering from a score of 2.8 in the 270 mesh operation plot to a score of 2.1 in the 80 mesh comparative plot.

Based on these results, the 270 mesh operation plot had the scores closer to those of the control plot in all the items than the 80 mesh comparative plot.

[Measurement Method for Blood Glucose Level]

For the measurement of the blood glucose level, comparison was made between the 80 mesh comparative plot and the 270 mesh operation plot with the control plot excluded. The measurement of the blood glucose level was performed in Shimadaya Corporation, and subjects were 4 healthy males and females over 20 years old including a person having a fasting blood glucose level corresponding to borderline diabetes. For cooking, 100 g corresponding to each serving of each sample was boiled for 5 minutes and 30 seconds in boiling water to be served as kake udon with no ingredients. Conditions were adjusted so that the udon was able to be finished in 5 minutes after serving, and the blood glucose level was measured 30 minutes, 60 minutes, 90 minutes and 120 minutes after starting the ingestion.

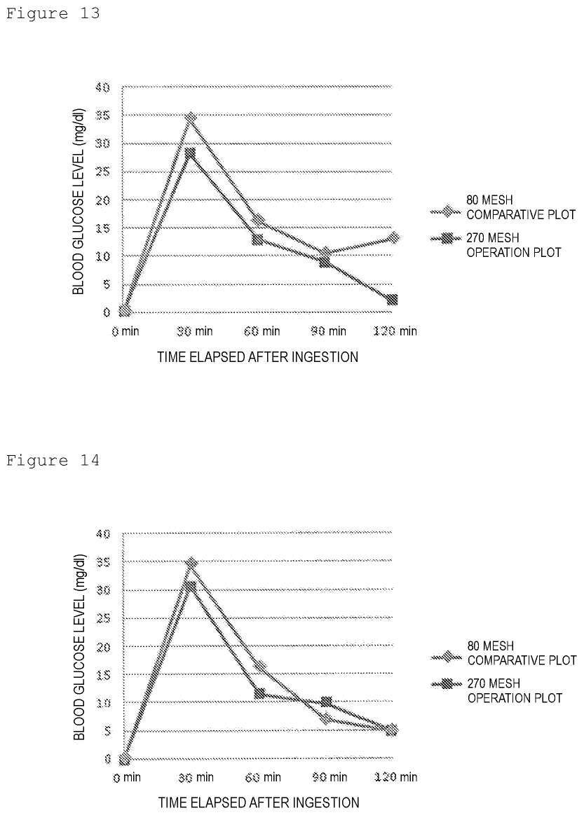

The thus obtained blood glucose level is shown in [Table 49], a change in the blood glucose level is shown in [Table 50], a difference (ΔCₘₐₓ) between a maximum blood glucose level and preprandial blood glucose level is shown in [Table 51], and a product (ΔAUC) of the blood glucose level and the time calculated using the preprandial blood glucose level as a baseline is shown in [Table 52]. Besides, transition of an average of the blood glucose levels is illustrated in a graph of FIG. 13.

TABLE 49
unit: mg/dl

| Time Elapsed | | 80 Mesh Comparative Plot | | | | | 270 Mesh Operation Plot | | | | |
|---|---|---|---|---|---|---|---|---|---|---|---|
| after Ingestion | | Prior | 30 min | 60 min | 90 min | 120 min | Prior | 30 min | 60 min | 90 min | 120 min |
| Subject | 1 | 94.5 | 143.5 | 112.5 | 111.0 | 114.5 | 99.0 | 144.0 | 112.0 | 112.0 | 108.5 |
| No | 2 | 103.5 | 137.0 | 132.5 | 100.5 | 116.5 | 102.0 | 124.5 | 117.5 | 109.5 | 104.5 |
| | 3 | 88.0 | 118.0 | 108.5 | 107.0 | 103.0 | 91.5 | 120.5 | 107.0 | 100.0 | 86.5 |
| | 4 | 90.5 | 115.5 | 88.0 | 100.0 | 94.0 | 97.5 | 113.5 | 105.0 | 104.5 | 99.0 |

| | | | | | | | | | | |
|---|---|---|---|---|---|---|---|---|---|---|
| Average of Subjects | 94.1 | 128.5 | 110.4 | 104.6 | 107.0 | 97.5 | 125.6 | 110.4 | 106.5 | 99.6 |

| | | | | | |
|---|---|---|---|---|---|
| T-test against 80 Mesh Comparative Plot (Probability | 15% | 45.2% | 100% | 62% | 21% |
| according to t-distribution) | | | | | |

TABLE 50
unit: mg/dl

| Time Elapsed | | 80 Mesh Comparative Plot | | | | 270 Mesh Operation Plot | | | |
|---|---|---|---|---|---|---|---|---|---|
| after Ingestion | | 30 min | 60 min | 90 min | 120 min | 30 min | 60 min | 90 min | 120 min |
| Subject | 1 | 49.0 | 18.0 | 16.5 | 20.0 | 45.0 | 13.0 | 13.0 | 9.5 |
| No. | 2 | 33.5 | 29.0 | −3.0 | 13.0 | 22.5 | 15.5 | 7.5 | 2.5 |
| | 3 | 30.0 | 20.5 | 19.0 | 15.0 | 29.0 | 15.5 | 8.5 | −5.0 |
| | 4 | 25.0 | −2.5 | 9.5 | 3.5 | 16.0 | 7.5 | 7.0 | 1.5 |

| | | | | | | | | |
|---|---|---|---|---|---|---|---|---|
| Average of Subjects | 34.4 | 16.3 | 10.5 | 12.9 | 28.1 | 12.9 | 9.0 | 2.1 |

| | | | | |
|---|---|---|---|---|
| T-test against 80 Mesh Comparative Plot (Probability | 7% | 54% | 75% | 6% |
| according to t-distribution) | | | | |

TABLE 51
unit: mg/dl

| | | |
|---|---|---|
| | 80 Mesh | 270 Mesh |
| Δ Cmax | Comparative Plot | Operation Plot |

| | | | |
|---|---|---|---|
| Subject No. | 1 | 49.0 | 45.0 |
| | 2 | 33.5 | 22.5 |
| | 3 | 30.0 | 29.0 |
| | 4 | 25.0 | 16.0 |

| | | |
|---|---|---|
| Average of Subjects | 34.4 | 28.1 |
| Standard Deviation | 10.4 | 12.4 |

| | |
|---|---|
| T-test against 80 Mesh Comparative Plot | 7.2% |
| (Probability according to t-distribution) | |

TABLE 52
unit: mg · min/dl

| | 80 Mesh | 270 Mesh |
|---|---|---|
| Δ AUC | Comparative Plot | Operation Plot |

| | | | |
|---|---|---|---|
| Subject No. | 1 | 2805 | 2273 |
| | 2 | 1980 | 1403 |
| | 3 | 2310 | 1515 |
| | 4 | 1013 | 938 |

| | | |
|---|---|---|
| Average of Subjects | 2027 | 1532 |
| Standard Deviation | 756 | 553 |

| | |
|---|---|
| T-test against 80 Mesh Comparative Plot | 4.7% |
| (Probability according to t-distribution) | |

[Measurement Results of Blood Glucose Level]

An average of the preprandial blood glucose levels of the 4 subjects was 94.1 to 97.5 mg/dl, ΔCₘₐₓ was 34.4 mg/dl in the 80 mesh comparative plot, but was an obviously low value of 28.1 mg/dl in the 270 mesh operation plot. Besides, ΔAUC was 2027 mg·min/dl in the 80 mesh comparative plot, but was an obviously low value of 1532 mg·min/dl in the 270 mesh operation plot, and a significant difference was found by a t-test.

In other words, it was confirmed that the effect of suppressing the blood glucose level increase can be obtained also in raw udon by using calcium alginate pulverized to 270 mesh pass as compared with that of 80 mesh pass.

### Example 11

<Raw Buckwheat Noodles>: The effect of suppressing the postprandial blood glucose level increase of raw buckwheat noodles in which Ca alginate was added was checked, and sensory evaluation was performed to compare 270 mesh pass and 80 mesh pass.

[Preparation of Raw Buckwheat Noodle Samples]

Powder raw materials shown in [Table 53] were respectively homogeneously mixed, and kneading water was respectively added thereto, followed by mixing. As a powder raw material, a wheat flour and a buckwheat flour (crude protein: 12.3%, ash: 1.6%) milled with chaff were mixed with vital gluten and an egg white powder, so as to enforce binding of a dough and improve texture. Ca alginate was added in a comparative plot and an operation plot, and an organic acid salt and salt were dissolved in the kneading water for purposes of improving the preserving property. The mixing was performed by kneading at a decompression degree of 80 kPa for 12 minutes to obtain a kneaded dough in a crumbled state at a temperature of 28 to 34° C.

The kneaded dough was formed into crude noodle belts each having a thickness of 6 mm using a noodle belt forming roll to be compounded with the thickness kept, the resultant was allowed to stand at 25° C. for 60 minutes under a condition where it was not dried, was rolled into a thickness of 1.45 mm using a four-stage flat roll, and then finely cut into noodle strings using cutting teeth having a grove width of 1.40 mm, and 100 g thereof corresponding to each serving was sealed with a polyethylene film after powder sprinkling.

TABLE 53

| | Sample Type | | | | | |
|---|---|---|---|---|---|---|
| | | | 80 Mesh | | 270 Mesh | |
| | | | Comparative | | Operation | |
| Item | Control | | Plot | | Plot | |
| (Powder Raw Materials) | | | | | | |
| Strong Wheat Flour | 1616 | g | 1616 | g | 1616 | g |
| Buckwheat Flour milled with Chaff | 1385 | g | 1385 | g | 1385 | g |
| Vital Gluten | 150 | g | 150 | g | 150 | g |
| Egg White Powder | 30 | g | 30 | g | 30 | g |
| 80 Mesh Pass Ca Alginate | | | 159 | g | | |
| 270 Mesh Pass Ca Alginate | | | | | 159 | g |
| Total of Powders | 3181 | g | 3340 | g | 3340 | g |
| (Kneading Water) | | | | | | |
| Organic Acid Salt | 46.2 | g | 46.2 | g | 46.2 | g |
| Salt | 63.3 | g | 76.8 | g | 76.8 | g |
| Fresh Water | 991 | g | 1202 | g | 1202 | g |

[Sensory Evaluation Test]

For cooking, 100 g corresponding to each serving of each sample was boiled in boiling water for 2 minutes. Sensory evaluation was performed on a 80 mesh comparative plot and a 270 mesh operation plot by using a control plot as a reference. By employing a 5-point rating scale assuming that the control plot had a score of 3, the four items of hardness (hard: 5 to soft: 1), viscoelasticity (viscoelastic: 5 to brittle: 1), powderiness (powdery: 5 to not powdery: 1), and comprehensive texture (preferable: 5 to not preferable: 1) were evaluated, and an average of scores of 4 specialized panelists was employed.

TABLE 54

| | | Hardness | | | Viscoelasticity | | |
|---|---|---|---|---|---|---|---|
| | | | 80 Mesh | 270 Mesh | | 80 Mesh | 270 Mesh |
| | | | Comparative | Operation | | Comparative | Operation |
| | | Control | Plot | Plot | Control | Plot | Plot |
| Evaluator No. | 1 | 3 | 3.0 | 3.0 | 3 | 3.0 | 3.0 |
| | | (reference) | | | (reference) | | |
| | 2 | 3 | 3.5 | 3.0 | 3 | 2.5 | 2.5 |
| | | (reference) | | | (reference) | | |
| | 3 | 3 | 4.0 | 3.5 | 3 | 2.0 | 2.5 |
| | | (reference) | | | (reference) | | |
| | 4 | 3 | 2.5 | 3.0 | 3 | 2.5 | 3.0 |
| | | (reference) | | | (reference) | | |
| Average | | | 3.3 | 3.1 | | 2.5 | 2.8 |
| Standard | | | 0.6 | 0.3 | | 0.4 | 0.3 |
| Deviation | | | | | | | |
| T-test against | | | | 63.8% | | | 18.2% |
| Comparative Plot | | | | | | | |
| (Probability | | | | | | | |
| according to t- | | | | | | | |
| distribution) | | | | | | | |
| | | Powderiness | | | Comprehensive Texture | | |
| | | | 80 Mesh | 270 Mesh | | 80 Mesh | 270 Mesh |
| | | | Comparative | Operation | | Comparative | Operation |
| | | Control | Plot | Plot | Control | Plot | Plot |
| Evaluator No. | 1 | 3 | 4.0 | 3.0 | 3 | 3.0 | 3.5 |
| | | (reference) | | | (reference) | | |
| | 2 | 3 | 3.5 | 3.0 | 3 | 2.5 | 3.0 |
| | | (reference) | | | (reference) | | |
| | 3 | 3 | 4.0 | 3.5 | 3 | 2.0 | 2.5 |
| | | (reference) | | | (reference) | | |
| | 4 | 3 | 4.0 | 3.0 | 3 | 2.5 | 3.0 |
| | | (reference) | | | (reference) | | |

| | | | | |
|---|---|---|---|---|
| Average | 3.9 | 3.1 | 2.5 | 3.0 |
| Standard | 0.3 | 0.3 | 0.4 | 0.4 |
| Deviation | | | | |

| | | |
|---|---|---|
| T-test against | 1.4% | 5%> |
| Comparative Plot | | |
| (Probability | | |
| according to t- | | |
| distribution) | | |

Results are shown in [Table 54]. As a result, in the item of hardness, as compared with the reference score of 3 of the control plot, there was a tendency of successively increasing from a score of 3.1 in the 270 mesh operation plot to a score of 3.3 in the 80 mesh comparative plot. In the item of viscoelasticity, as compared with the reference score of 3 of the control plot, there was a tendency successively lowering from a score of 2.8 in the 270 mesh operation plot to a score of 2.5 in the 80 mesh comparative plot. In the item of powderiness, as compared with the reference score of 3 of the control plot, there was a tendency of successively increasing from a score of 3.1 in the 270 mesh operation plot to a score of 3.9 in the 80 mesh comparative plot. In the item of comprehensive texture, as compared with the reference score of 3 of the control plot, the 270 mesh operation plot had the same score and the 80 mesh comparative plot had a low score of 2.5.

Based on these results, the 270 mesh operation plot had the scores closer to those of the control plot in all the items than the 80 mesh comparative plot.

[Measurement Method for Blood Glucose Level]

For the measurement of the blood glucose level, comparison was made between the 80 mesh comparative plot and the 270 mesh operation plot with the control plot excluded. The measurement of the blood glucose level was performed in Shimadaya Corporation, and subjects were 4 healthy males and females over 20 years old including a person having a fasting blood glucose level corresponding to borderline diabetes. For cooking, 100 g corresponding to each serving of each sample was boiled for 2 minutes in boiling water to be served as kake soba with no ingredients. Conditions were adjusted so that the buckwheat noodles were able to be finished in 5 minutes after serving, and the blood glucose level was measured 30 minutes, 60 minutes, 90 minutes and 120 minutes after starting the ingestion.

The thus obtained blood glucose level is shown in [Table 55], a change in the blood glucose level is shown in [Table 56], a difference (ΔCₘₐₓ) between a maximum blood glucose level and preprandial blood glucose level is shown in [Table 57], and a product (ΔAUC) of the blood glucose level and the time calculated using the preprandial blood glucose level as a baseline is shown in [Table 58]. Besides, transition of an average of the blood glucose levels is illustrated in a graph of FIG. 14.

TABLE 55
unit: mg/dl

| Time Elapsed | | 80 Mesh Comparative Plot | | | | | 270 Mesh Operation Plot | | | | |
|---|---|---|---|---|---|---|---|---|---|---|---|
| after Ingestion | | Prior | 30 min | 60 min | 90 min | 120 min | Prior | 30 min | 60 min | 90 min | 120 min |
| Subject | 1 | 94.0 | 137.5 | 115.0 | 105.0 | 102.0 | 97.0 | 135.0 | 107.0 | 112.5 | 104.5 |
| No | 2 | 100.5 | 133.0 | 124.5 | 103.5 | 100.5 | 99.0 | 126.5 | 109.5 | 111.5 | 99.5 |
| | 3 | 93.5 | 125.0 | 108.0 | 104.5 | 102.0 | 88.5 | 122.5 | 100.0 | 91.0 | 92.0 |
| | 4 | 94.0 | 123.5 | 99.5 | 96.5 | 96.0 | 93.0 | 116.0 | 107.0 | 101.5 | 100.5 |

| | | | | | | | | | | |
|---|---|---|---|---|---|---|---|---|---|---|
| Average of Subjects | 95.5 | 129.8 | 111.8 | 102.4 | 100.1 | 94.4 | 125.0 | 105.9 | 104.1 | 99.1 |

| | | | | | |
|---|---|---|---|---|---|
| T-test against 80 Mesh Comparative Plot (Probability | 54% | 3.6% | 30% | 76% | 78% |
| according to t-distribution) | | | | | |

TABLE 56
unit: mg/dl

| Time Elapsed | | 80 Mesh Comparative Plot | | | | 270 Mesh Operation Plot | | | |
|---|---|---|---|---|---|---|---|---|---|
| after Ingestion | | 30 min | 60 min | 90 min | 120 min | 30 min | 60 min | 90 min | 120 min |
| Subject | 1 | 43.5 | 21.0 | 11.0 | 8.0 | 38.0 | 10.0 | 15.5 | 7.5 |
| No. | 2 | 32.5 | 24.0 | 3.0 | 0.0 | 27.5 | 10.5 | 12.5 | 0.5 |
| | 3 | 31.5 | 14.5 | 11.0 | 8.5 | 34.0 | 11.5 | 2.5 | 3.5 |
| | 4 | 29.5 | 5.5 | 2.5 | 2.0 | 23.0 | 14.0 | 8.5 | 7.5 |

| | | | | | | | | |
|---|---|---|---|---|---|---|---|---|
| Average of Subjects | 34.3 | 16.3 | 6.9 | 4.6 | 30.6 | 11.5 | 9.8 | 4.8 |

| | | | | |
|---|---|---|---|---|
| T-test against 80 Mesh Comparative Plot (Probability | 18% | 41% | 52% | 96% |
| according to t-distribution) | | | | |

TABLE 57
unit: mg/dl

| | 80 Mesh | 270 Mesh |
|---|---|---|
| Δ Cmax | Comparative Plot | Operation Plot |

| | | | |
|---|---|---|---|
| Subject No. | 1 | 43.5 | 38.0 |
| | 2 | 32.5 | 27.5 |
| | 3 | 31.5 | 34.0 |
| | 4 | 29.5 | 23.0 |

| | | |
|---|---|---|
| Average of Subjects | 34.3 | 30.6 |
| Standard Deviation | 6.3 | 6.7 |

| | |
|---|---|
| T-test against 80 Mesh Comparative Plot | 17.7% |
| (Probability according to t-distribution) | |

TABLE 58
unit: mg · min/dl

| | 80 Mesh | 270 Mesh |
|---|---|---|
| Δ AUC | Comparative Plot | Operation Plot |

| | | | |
|---|---|---|---|
| Subject No. | 1 | 2385 | 2018 |
| | 2 | 1785 | 1523 |
| | 3 | 1838 | 1493 |
| | 4 | 1155 | 1478 |

| | | |
|---|---|---|
| Average of Subjects | 1791 | 1628 |
| Standard Deviation | 503 | 261 |

| | |
|---|---|
| T-test against 80 Mesh Comparative Plot | 39.2% |
| (Probability according to t-distribution) | |

[Measurement Results of Blood Glucose Level]

An average of the preprandial blood glucose levels of the 4 subjects was 94.4 to 95.5 mg/dl, ΔCₘₐₓ was 34.3 mg/dl in the 80 mesh comparative plot, but was a low value of 30.6 mg/dl in the 270 mesh operation plot. Besides, ΔAUC was 1791 mg·min/dl in the 80 mesh comparative plot, but was a low value of 1628 mg·min/dl in the 270 mesh operation plot.

In other words, it was confirmed that the effect of suppressing the blood glucose level increase can be obtained also in raw buckwheat noodles by using calcium alginate pulverized to 270 mesh pass as compared with that of 80 mesh pass.

### Example 12

<Raw Pasta>: The effect of suppressing the postprandial blood glucose level increase of raw pasta in which Ca alginate was added was checked, and sensory evaluation was performed to compare 270 mesh pass and 80 mesh pass.

[Preparation of Raw Pasta Samples]

Powder raw materials shown in [Table 59] were respectively homogeneously mixed, and kneading water was respectively added thereto, followed by mixing. Ca alginate was added in a comparative plot and an operation plot, and salt and a pigment were dissolved in the kneading water in addition to alcohol used for purposes of improving the preserving property. The mixing was performed by kneading at a decompression degree of 80 kPa for 12 minutes to obtain a kneaded dough in a crumbled state at a temperature of 28 to 34° C.

The kneaded dough was formed into crude noodle belts each having a thickness of 8 mm using a noodle belt forming roll to be compounded with the thickness kept, the resultant was allowed to stand at 25° C. for 60 minutes under a condition where it was not dried, was rolled into a thickness of 1.95 mm using a four-stage flat roll, and then finely cut into noodle strings using cutting teeth having a grove width of 1.50 mm, and 100 g thereof corresponding to each serving was sealed with a polyethylene film after powder sprinkling.

TABLE 59

| | Sample Type | | | | | |
|---|---|---|---|---|---|---|
| | | | 80 Mesh | | 270 Mesh | |
| | | | Comparative | | Operation | |
| Item | Control | | Plot | | Plot | |
| (Powder Raw Materials) | | | | | | |
| All Purpose Flour | 3000 | g | 3000 | g | 3000 | g |
| Egg Yolk Powder | 90 | g | 90 | g | 90 | g |
| 80 Mesh Pass Ca Alginate | | | 156 | g | | |
| 270 Mesh Pass Ca Alginate | | | | | 156 | g |
| Total of Powders | 3090 | g | 3246 | g | 3246 | g |
| (Kneading Water) | | | | | | |
| Alcohol | 150 | g | 150 | g | 150 | g |
| Pigment | 0.75 | g | 0.75 | g | 0.75 | g |
| Salt | 83.3 | g | 109 | g | 109 | g |
| Fresh Water | 750 | g | 981 | g | 981 | g |

[Sensory Evaluation Test]

For cooking, each sample was boiled in boiling water for 2 minutes and 30 seconds. Sensory evaluation was performed on a 80 mesh comparative plot and a 270 mesh operation plot by using a control plot as a reference. By employing a 5-point rating scale assuming that raw pasta of the control plot had a score of 3, the four items of hardness (hard: 5 to soft: 1), viscoelasticity (viscoelastic: 5 to brittle: 1), powderiness (powdery: 5 to not powdery: 1), and comprehensive texture (preferable: 5 to not preferable: 1) were evaluated, and an average of scores of 4 specialized panelists was employed.

TABLE 60

| | | Hardness | | | Viscoelasticity | | |
|---|---|---|---|---|---|---|---|
| | | | 80 Mesh | 270 Mesh | | 80 Mesh | 270 Mesh |
| | | | Comparative | Operation | | Comparative | Operation |
| | | Control | Plot | Plot | Control | Plot | Plot |
| Evaluator No. | 1 | 3.0 | 2.0 | 2.5 | 3.0 | 2.0 | 2.0 |
| | | (reference) | | | (reference) | | |
| | 2 | 3.0 | 2.5 | 3.0 | 3.0 | 3.0 | 3.0 |
| | | (reference) | | | (reference) | | |
| | 3 | 3.0 | 2.5 | 3.0 | 3.0 | 2.5 | 3.0 |
| | | (reference) | | | (reference) | | |
| | 4 | 3.0 | 3.0 | 3.0 | 3.0 | 3.5 | 3.5 |
| | | (reference) | | | (reference) | | |
| Average | | | 2.5 | 2.9 | | 2.8 | 2.9 |
| Standard | | | 0.4 | 0.3 | | 0.6 | 0.6 |
| Deviation | | | | | | | |
| T-test against | | | | 5.8% | | | 39.1% |
| Comparative Plot | | | | | | | |
| (Probability | | | | | | | |
| according to t- | | | | | | | |
| distribution) | | | | | | | |
| | | Powderiness | | | Comprehensive Texture | | |
| | | | 80 Mesh | 270 Mesh | | 80 Mesh | 270 Mesh |
| | | | Comparative | Operation | | Comparative | Operation |
| | | Control | Plot | Plot | Control | Plot | Plot |
| Evaluator No. | 1 | 3.0 | 3.0 | 3.0 | 3.0 | 2.5 | 2.5 |
| | | (reference) | | | (reference) | | |
| | 2 | 3.0 | 3.0 | 3.0 | 3.0 | 3.0 | 3.0 |
| | | (reference) | | | (reference) | | |
| | 3 | 3.0 | 3.5 | 3.5 | 3.0 | 2.5 | 3.0 |
| | | (reference) | | | (reference) | | |
| | 4 | 3.0 | 3.5 | 3.0 | 3.0 | 3.5 | 3.5 |
| | | (reference) | | | (reference) | | |

| | | | | |
|---|---|---|---|---|
| Average | 3.3 | 3.1 | 2.9 | 3.0 |
| Standard | 0.3 | 0.3 | 0.5 | 0.4 |
| Deviation | | | | |

| | | |
|---|---|---|
| T-test against | 39.1% | 39.1% |
| Comparative Plot | | |
| (Probability | | |
| according to t- | | |
| distribution) | | |

Results are shown in [Table 60]. As a result, in the item of hardness, as compared with the reference score of 3 of the control plot, there was a tendency of successively lowering from a score of 2.9 in the 270 mesh operation plot to a score of 2.5 in the 80 mesh comparative plot. In the item of viscoelasticity, as compared with the reference score of 3 of the control plot, there was a tendency of successively lowering from a score of 2.9 in the 270 mesh operation plot to a score of 2.8 in the 80 mesh comparative plot. In the item of powderiness, as compared with the reference score of 3 of the control plot, there was a tendency of successively increasing from a score of 3.1 in the 270 mesh operation plot to a score of 3.3 in the 80 mesh comparative plot. In the item of comprehensive texture, as compared with the reference score of 3 of the control plot, the 270 mesh operation plot had the same score and the 80 mesh comparative plot had a score of a low value of 2.9.

Based on these results, the 270 mesh operation plot had the scores closer to those of the control plot in all the items than the 80 mesh comparative plot.

[Measurement Method for Blood Glucose Level]

For the measurement of the blood glucose level, comparison was made between the 80 mesh comparative plot and the 270 mesh operation plot with the control plot excluded. The measurement of the blood glucose level was performed in Shimadaya Corporation, and subjects were 4 healthy males and females over 20 years old including a person having a fasting blood glucose level corresponding to borderline diabetes. For cooking, 100 g corresponding to each serving of each sample was boiled for 2 minutes and 30 seconds in boiling water to be served after being dressed with 15 g of a commercially available pasta seasoning with no ingredients. Conditions were adjusted so that the pasta was able to be finished in 5 minutes after serving, and the blood glucose level was measured 30 minutes, 60 minutes, 90 minutes and 120 minutes after starting the ingestion.

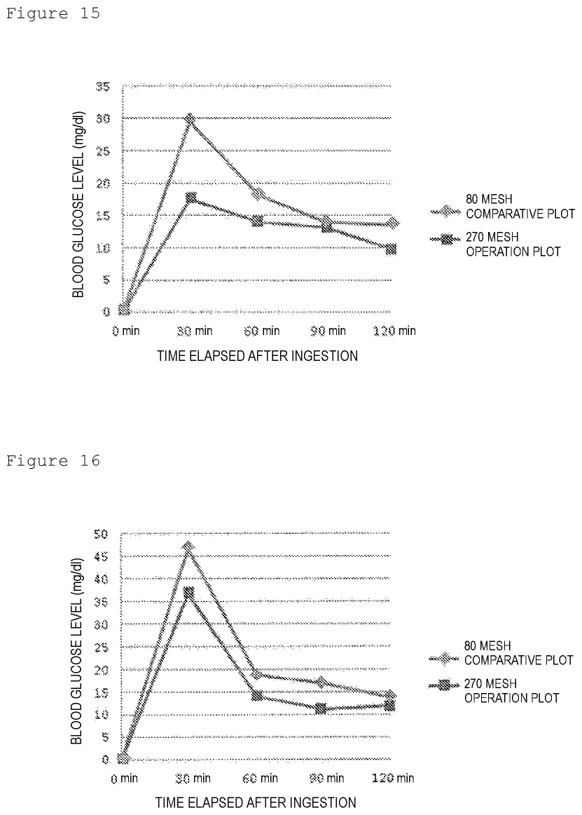

The thus obtained blood glucose level is shown in [Table 61], a change in the blood glucose level is shown in [Table 62], a difference (ΔCₘₐₓ) between a maximum blood glucose level and preprandial blood glucose level is shown in [Table 63], and a product (ΔAUC) of the blood glucose level and the time calculated using the preprandial blood glucose level as a baseline is shown in [Table 64]. Besides, transition of an average of the blood glucose levels is illustrated in a graph of FIG. 15.

TABLE 61
unit: mg/dl

| Time Elapsed | | 80 Mesh Comparative Plot | | | | | 270 Mesh Operation Plot | | | | |
|---|---|---|---|---|---|---|---|---|---|---|---|
| after Ingestion | | Prior | 30 min | 60 min | 90 min | 120 min | Prior | 30 min | 60 min | 90 min | 120 min |
| Subject | 1 | 95.0 | 138.0 | 101.5 | 107.5 | 119.5 | 91.5 | 115.5 | 110.5 | 109.5 | 105.5 |
| No | 2 | 104.0 | 129.0 | 124.0 | 119.0 | 117.5 | 106.5 | 122.0 | 122.0 | 116.5 | 112.0 |
| | 3 | 87.0 | 109.5 | 126.5 | 112.5 | 95.5 | 84.5 | 104.5 | 100.5 | 93.0 | 90.5 |
| | 4 | 89.5 | 117.0 | 96.5 | 92.5 | 97.0 | 95.0 | 105.5 | 100.5 | 111.0 | 108.5 |

| | | | | | | | | | | |
|---|---|---|---|---|---|---|---|---|---|---|
| Average of Subjects | 93.9 | 123.4 | 112.1 | 107.9 | 107.4 | 94.4 | 111.9 | 108.4 | 107.5 | 104.1 |

| | | | | | |
|---|---|---|---|---|---|
| T-test against 80 Mesh Comparative Plot (Probability | 83% | 6.0% | 66% | 96% | 59% |
| according to t-distribution) | | | | | |

TABLE 62
unit: mg/dl

| Time Elapsed | | 80 Mesh Comparative Plot | | | | 270 Mesh Operation Plot | | | |
|---|---|---|---|---|---|---|---|---|---|
| after Ingestion | | 30 min | 60 min | 90 min | 120 min | 30 min | 60 min | 90 min | 120 min |
| Subject | 1 | 43.0 | 6.5 | 12.5 | 24.5 | 24.0 | 19.0 | 18.0 | 14.0 |
| No. | 2 | 25.0 | 20.0 | 15.0 | 13.5 | 15.5 | 15.5 | 10.0 | 5.5 |
| | 3 | 22.5 | 39.5 | 25.5 | 8.5 | 20.0 | 16.0 | 8.5 | 6.0 |
| | 4 | 27.5 | 7.0 | 3.0 | 7.5 | 10.5 | 5.5 | 16.0 | 13.5 |

| | | | | | | | | |
|---|---|---|---|---|---|---|---|---|
| Average of Subjects | 29.5 | 18.3 | 14.0 | 13.5 | 17.5 | 14.0 | 13.1 | 9.8 |

| | | | | |
|---|---|---|---|---|
| T-test against 80 Mesh Comparative Plot (Probability | 5% | 61% | 90% | 38% |
| according to t distribution) | | | | |

TABLE 63
unit: mg/dl

| | 80 Mesh | 270 Mesh |
|---|---|---|
| Δ Cmax | Comparative Plot | Operation Plot |

| | | | |
|---|---|---|---|
| Subject No. | 1 | 43.0 | 24.0 |
| | 2 | 25.0 | 15.5 |
| | 3 | 39.5 | 20.0 |
| | 4 | 27.5 | 16.0 |

| | | |
|---|---|---|
| Average of Subjects | 33.8 | 18.9 |
| Standard Deviation | 8.8 | 4.0 |

| | |
|---|---|
| T-test against 80 Mesh Comparative Plot | 1.0% |
| (Probability according to t-distribution) | |

TABLE 64
unit: mg · min/dl

| | 80 Mesh | 270 Mesh |
|---|---|---|
| Δ AUC | Comparative Plot | Operation Plot |

| | | | |
|---|---|---|---|
| Subject No. | 1 | 2228 | 2040 |
| | 2 | 2003 | 1313 |
| | 3 | 2753 | 1425 |
| | 4 | 1238 | 1163 |

| | | |
|---|---|---|
| Average of Subjects | 2055 | 1485 |
| Standard Deviation | 629 | 385 |

| | |
|---|---|
| T-test against 80 Mesh Comparative Plot | 14.0% |
| (Probability according to t-distribution) | |

[Measurement Results of Blood Glucose Level]

An average of the preprandial blood glucose levels of the 4 subjects was 93.9 to 94.4 mg/dl, ΔCₘₐₓ was 33.8 mg/dl in the 80 mesh comparative plot, but was an obviously low value of 18.9 mg/dl in the 270 mesh operation plot, and a significant difference was found by a t-test.

Besides, ΔAUC was 2055 mg·min/dl in the 80 mesh comparative plot, but was an obviously low value of 1485 mg·min/dl in the 270 mesh operation plot.

In other words, it was confirmed that the effect of suppressing the blood glucose level increase can be obtained also in raw pasta by using calcium alginate pulverized to 270 mesh pass as compared with that of 80 mesh pass.

### Example 13

<Raw Chinese Noodles>: The effect of suppressing the postprandial blood glucose level increase of raw Chinese noodles in which Ca alginate was added was checked, and sensory evaluation was performed to compare 270 mesh pass and 80 mesh pass.

[Preparation of Raw Chinese Noodle Samples]

Powder raw materials shown in [Table 65] were respectively homogeneously mixed, and kneading water was respectively added thereto, followed by mixing. As a powder raw material, a wheat flour for Chinese noodles was mixed with vital gluten, so as to improve texture and enforce binding of a dough. Ca alginate was added in a comparative plot and an operation plot, and kansui, salt and a pigment were dissolved in the kneading water in addition to alcohol used for purposes of improving the preserving property. The mixing was performed by kneading at a decompression degree of 80 kPa for 12 minutes to obtain a kneaded dough in a crumbled state at a temperature of 28 to 34° C.

The kneaded dough was formed into crude noodle belts each having a thickness of 6 mm using a noodle belt forming roll to be compounded with the thickness kept, the resultant was allowed to stand at 25° C. for 60 minutes under a condition where it was not dried, was rolled into a thickness of 1.5 mm using a four-stage flat roll, and then finely cut into noodle strings using cutting teeth having a grove width of 1.4 mm, and 100 g thereof corresponding to each serving was sealed with a polyethylene film after powder sprinkling.

TABLE 65

| | Sample Type | | | | | |
|---|---|---|---|---|---|---|
| | | | 80 Mesh | | 270 Mesh | |
| | | | Comparative | | Operation | |
| Item | Control | | Plot | | Plot | |
| (Powder Raw Materials) | | | | | | |
| Wheat Flour | 3000 | g | 3000 | g | 3000 | g |
| Vital Gluten | 90 | g | 90 | g | 90 | g |
| 80 Mesh Pass Ca Alginate | | | 156 | g | | |
| 270 Mesh Pass Ca Alginate | | | | | 156 | g |
| Total of Powders | 3090 | g | 3246 | g | 3246 | g |
| (Kneading Water) | | | | | | |
| Alcohol | 135 | g | 135 | g | 135 | g |
| Kansui (Powder) | 30.3 | g | 30.3 | g | 30.3 | g |
| Salt | 54 | g | 54 | g | 54 | g |
| Gardenia Pigment | 2.7 | g | 2.7 | g | 2.7 | g |
| Fresh Water | 780 | g | 1020 | g | 1020 | g |

[Sensory Evaluation Test]

For cooking, each sample was boiled in boiling water for 2 minutes and 30 seconds. Sensory evaluation was performed on a 80 mesh comparative plot and a 270 mesh operation plot by using a control plot as a reference. By employing a 5-point rating scale assuming that the control plot had a score of 3, the four items of hardness (hard: 5 to soft: 1), viscoelasticity (viscoelastic: 5 to brittle: 1), powderiness (powdery: 5 to not powdery: 1), and comprehensive texture (preferable: 5 to not preferable: 1) were evaluated, and an average of scores of 4 specialized panelists was employed.

TABLE 66

| | | Hardness | | | Viscoelasticity | | |
|---|---|---|---|---|---|---|---|
| | | | 80 Mesh | 270 Mesh | | 80 Mesh | 270 Mesh |
| | | | Comparative | Operation | | Comparative | Operation |
| | | Control | Plot | Plot | Control | Plot | Plot |
| Evaluator No. | 1 | 3 | 2.0 | 2.0 | 3 | 3.5 | 3.5 |
| | | (reference) | | | (reference) | | |
| | 2 | 3 | 2.5 | 2.5 | 3 | 2.5 | 3.0 |
| | | (reference) | | | (reference) | | |
| | 3 | 3 | 2.0 | 2.5 | 3 | 2.5 | 3.0 |
| | | (reference) | | | (reference) | | |
| | 4 | 3 | 2.0 | 2.5 | 3 | 1.5 | 2.5 |
| | | (reference) | | | (reference) | | |
| Average | | | 2.1 | 2.4 | | 2.5 | 3.0 |
| Standard | | | 0.3 | 0.3 | | 0.8 | 0.4 |
| Deviation | | | | | | | |
| T-test against | | | | 18.2% | | | 9.2% |
| Comparative Plot | | | | | | | |
| (Probability | | | | | | | |
| according to t- | | | | | | | |
| distribution) | | | | | | | |
| | | Powderiness | | | Comprehensive Texture | | |
| | | | 80 Mesh | 270 Mesh | | 80 Mesh | 270 Mesh |
| | | | Comparative | Operation | | Comparative | Operation |
| | | Control | Plot | Plot | Control | Plot | Plot |
| Evaluator No. | 1 | 3 | 3.0 | 3.0 | 3 | 2.5 | 2.5 |
| | | (reference) | | | (reference) | | |
| | 2 | 3 | 4.0 | 3.5 | 3 | 2.0 | 2.5 |
| | | (reference) | | | (reference) | | |
| | 3 | 3 | 4.0 | 3.5 | 3 | 2.5 | 2.5 |
| | | (reference) | | | (reference) | | |
| | 4 | 3 | 2.5 | 2.5 | 3 | 2.0 | 2.5 |
| | | (reference) | | | (reference) | | |

| | | | | |
|---|---|---|---|---|
| Average | 3.4 | 3.1 | 2.3 | 2.5 |
| Standard | 0.8 | 0.5 | 0.3 | 0.0 |
| Deviation | | | | |

| | | |
|---|---|---|
| T-test against | 18.2% | 18.2% |
| Comparative Plot | | |
| (Probability | | |
| according to t- | | |
| distribution) | | |

Results are shown in [Table 66]. As a result, in the item of hardness, as compared with the reference score of 3 of the control plot, there was a tendency of successively lowering from a score of 2.4 in the 270 mesh operation plot to a score of 2.1 in the 80 mesh comparative plot. In the item of viscoelasticity, as compared with the reference score of 3 of the control plot, the 270 mesh operation plot had the same score and the 80 mesh comparative plot had a score of a low value of 2.5. In the item of powderiness, as compared with the reference score of 3 of the control plot, there was a tendency of successively increasing from a score of 3.1 in the 270 mesh operation plot to a score of 3.4 in the 80 mesh comparative plot. In the item of comprehensive texture, as compared with the reference score of 3 of the control plot, there was a tendency of successively lowering from a score of 2.5 in the 270 mesh operation plot to a score of 2.3 in the 80 mesh comparative plot.

Based on these results, the 270 mesh operation plot had the scores closer to those of the control plot in all the items than the 80 mesh comparative plot.

[Measurement Method for Blood Glucose Level]

For the measurement of the blood glucose level, comparison was made between the 80 mesh comparative plot and the 270 mesh operation plot with the control plot excluded. The measurement of the blood glucose level was performed in Shimadaya Corporation, and subjects were 4 healthy males and females over 20 years old including a person having a fasting blood glucose level corresponding to borderline diabetes.

For cooking, 100 g corresponding to each serving of each sample was boiled for 2 minutes and 30 seconds in boiling water to be served as ramen in a soy-sauce flavored soup with no ingredients. Conditions were adjusted so that the ramen was able to be finished in 5 minutes after serving, and the blood glucose level was measured 30 minutes, 60 minutes, 90 minutes and 120 minutes after starting the ingestion.

The thus obtained blood glucose level is shown in [Table 67], a change in the blood glucose level is shown in [Table 68], a difference (ΔCₘₐₓ) between a maximum blood glucose level and preprandial blood glucose level is shown in [Table 69], and a product (ΔAUC) of the blood glucose level and the time calculated using the preprandial blood glucose level as a baseline is shown in [Table 70]. Besides, transition of an average of the blood glucose levels is illustrated in a graph of FIG. 16.

TABLE 67
unit: mg/dl

| Time Elapsed | | 80 Mesh Comparative Plot | | | | | 270 Mesh Operation Plot | | | | |
|---|---|---|---|---|---|---|---|---|---|---|---|
| after Ingestion | | Prior | 30 min | 60 min | 90 min | 120 mm | Prior | 30 min | 60 min | 90 min | 120 mm |
| Subject | 1 | 90.5 | 165.5 | 121.0 | 107.0 | 114.5 | 88.0 | 137.0 | 104.0 | 104.0 | 101.5 |
| No. | 2 | 97.5 | 139.5 | 113.5 | 115.5 | 103.5 | 100.5 | 128.5 | 113.0 | 110.5 | 107.0 |
| | 3 | 79.0 | 112.5 | 97.5 | 101.5 | 89.5 | 86.0 | 122.0 | 114.0 | 96.5 | 106.5 |
| | 4 | 89.5 | 126.0 | 97.0 | 100.5 | 104.5 | 98.0 | 131.5 | 98.0 | 106.5 | 104.5 |

| | | | | | | | | | | |
|---|---|---|---|---|---|---|---|---|---|---|
| Average of Subjects | 89.1 | 135.9 | 108.0 | 106.1 | 103.0 | 93.1 | 129.8 | 107.3 | 104.4 | 104.9 |

| | | | | | |
|---|---|---|---|---|---|
| T-test against 80 Mesh Comparative Plot (Probability | 20% | 53.1% | 93% | 55% | 78% |
| according to t-distribution) | | | | | |

TABLE 68
unit: mg/dl

| Time Elapsed | | 80 Mesh Comparative Plot | | | | 270 Mesh Operation Plot | | | |
|---|---|---|---|---|---|---|---|---|---|
| after Ingestion | | 30 min | 60 min | 90 min | 120 min | 30 min | 60 min | 90 min | 120 min |
| Subject | 1 | 75.0 | 33.5 | 16.5 | 24.0 | 49.0 | 16.0 | 16.0 | 13.5 |
| No. | 2 | 42.0 | 16.0 | 18.0 | 6.0 | 28.0 | 12.5 | 10.0 | 6.5 |
| | 3 | 33.5 | 18.5 | 22.5 | 10.5 | 36.0 | 28.0 | 10.5 | 20.5 |
| | 4 | 36.5 | 7.5 | 11.0 | 15.0 | 33.5 | 0.0 | 8.5 | 6.5 |

| | | | | | | | | |
|---|---|---|---|---|---|---|---|---|
| Average of Subjects | 46.8 | 18.9 | 17.0 | 13.9 | 36.6 | 14.1 | 11.3 | 11.8 |

| | | | | |
|---|---|---|---|---|
| T-test against 80 Mesh Comparative Plot (Probability | 21% | 46% | 12% | 68% |
| according to t-distribution) | | | | |

TABLE 69
unit: mg/dl

| | 80 Mesh | 270 Mesh |
|---|---|---|
| Δ Cmax | Comparative Plot | Operation Plot |

| | | | |
|---|---|---|---|
| Subject No. | 1 | 75.0 | 49.0 |
| | 2 | 42.0 | 28.0 |
| | 3 | 33.5 | 36.0 |
| | 4 | 36.5 | 33.5 |

| | | |
|---|---|---|
| Average of Subjects | 46.8 | 36.6 |
| Standard Deviation | 19.2 | 8.9 |

| | |
|---|---|
| T-test against 80 Mesh Comparative Plot | 20.7% |
| (Probability according to t-distribution) | |

TABLE 70
unit: mg · min/dl

| | 80 Mesh | 270 Mesh |
|---|---|---|
| Δ AUC | Comparative Plot | Operation Plot |

| | | | |
|---|---|---|---|
| Subject No. | 1 | 4110 | 2633 |
| | 2 | 2370 | 1613 |
| | 3 | 2393 | 2543 |
| | 4 | 1875 | 1358 |

| | | |
|---|---|---|
| Average of Subjects | 2687 | 2036 |
| Standard Deviation | 978 | 646 |

| | |
|---|---|
| T-test against 80 Mesh Comparative Plot | 14.8% |
| (Probability according to t-distribution) | |

[Measurement Results of Blood Glucose Level]

An average of the preprandial blood glucose levels of the 4 subjects was 89.1 to 93.1 mg/dl, ΔCₘₐₓ was 46.8 mg/dl in the 80 mesh comparative plot, but was a low value of 36.6 mg/dl in the 270 mesh operation plot. Besides, ΔAUC was 2687 mg·min/dl in the 80 mesh comparative plot, but was a low value of 2036 mg·min/dl in the 270 mesh operation plot.

In other words, it was confirmed that the effect of suppressing the blood glucose level increase can be obtained also in raw Chinese noodles by using calcium alginate pulverized to 270 mesh pass as compared with that of 80 mesh pass.

### Example 14

<Frozen Boiled Pasta>: The effect of suppressing the postprandial blood glucose level increase of boiled pasta in which Ca alginate was added was checked, and sensory evaluation was performed to compare 270 mesh pass and 80 mesh pass.

[Preparation of Boiled Pasta Samples]

Powder raw materials shown in [Table 71] were respectively homogeneously mixed, and kneading water was respectively added thereto, followed by mixing. Ca alginate was added in a comparative plot and an operation plot, and a salt solution was used as the kneading water for purposes of improving the noodle making properties. The mixing was performed by kneading at a decompression degree of 90 kPa for 8 minutes to obtain a kneaded dough in a crumbled state at a temperature of 28 to 34° C. The kneaded dough was formed into crude noodle belts each having a thickness of 9 mm using a noodle belt forming roll to be compounded with the thickness kept, the resultant was allowed to stand at 25° C. for 60 minutes under a condition where it was not dried, was rolled into a thickness of 2.15 mm using a four-stage flat roll, and then finely cut into noodle strings using cutting teeth having a grove width of 1.9 mm.

The thus obtained noodle strings were boiled in hot water for boiling at 98° C. for 1 minute, then somewhat cooled by putting them in water at 15° C. twice, and then cooled with ice water at 5° C. for 1 minute. Thereafter, the solution remaining thereon was rapidly removed, 150 g thereof corresponding to each serving was put in a forming tray for freezing, and was quick-frozen at −36° C. for 40 minutes to prepare frozen boiled pasta having a boiled noodle moisture shown in [Table 71].

TABLE 71

| | Sample Type | | | | | |
|---|---|---|---|---|---|---|
| | | | 80 Mesh | | 270 Mesh | |
| | | | Comparative | | Operation | |
| Item | Control | | Plot | | Plot | |
| (Powder Raw Materials) | | | | | | |
| Durum Wheat Flour | 2571 | g | 2571 | g | 2571 | g |
| Acetylated Tapioca Starch | 429 | g | 429 | g | 429 | g |
| 80 Mesh Pass Ca Alginate | | | 150 | g | | |
| 270 Mesh Pass Ca Alginate | | | | | 150 | g |
| Total of Powders | 3000 | g | 3150 | g | 3150 | g |
| (Kneading Water) | | | | | | |
| Salt | 68.7 | g | 68.7 | g | 68.7 | g |
| Fresh Water | 1182 | g | 1440 | g | 1440 | g |
| Boiling Time | 1 | min | 1 | min | 1 | min |

| | | | |
|---|---|---|---|
| * Boiled Noodle Average | 62.5% | 64.7% | 64.7% |
| Moisture | | | |

[Sensory Evaluation Test]

For cooking, each sample was boiled in boiling water for 20 to 30 seconds. Sensory evaluation was performed on a 80 mesh comparative plot and a 270 mesh operation plot by using a control plot as a reference. By employing a 5-point rating scale assuming that boiled pasta of the control plot had a score of 3, the four items of hardness (hard: 5 to soft: 1), viscoelasticity (viscoelastic: 5 to brittle: 1), powderiness (powdery: 5 to not powdery: 1), and comprehensive texture (preferable: 5 to not preferable: 1) were evaluated, and an average of scores of 5 specialized panelists was employed.

TABLE 72

| | | Hardness | | | Viscoelasticity | | |
|---|---|---|---|---|---|---|---|
| | | | 80 Mesh | 270 Mesh | | 80 Mesh | 270 Mesh |
| | | | Comparative | Operation | | Comparative | Operation |
| | | Control | Plot | Plot | Control | Plot | Plot |
| Evaluator No. | 1 | 3.0 | 2.5 | 2.5 | 3.0 | 3.5 | 3.5 |
| | | (reference) | | | (reference) | | |
| | 2 | 3.0 | 3.0 | 3.0 | 3.0 | 2.5 | 3.0 |
| | | (reference) | | | (reference) | | |
| | 3 | 3.0 | 2.0 | 2.5 | 3.0 | 2.0 | 2.5 |
| | | (reference) | | | (reference) | | |
| | 4 | 3.0 | 2.5 | 2.5 | 3.0 | 3.0 | 3.0 |
| | | (reference) | | | (reference) | | |
| | 5 | 3.0 | 3.5 | 3.5 | 3.0 | 5.0 | 4.0 |
| | | (reference) | | | (reference) | | |
| Average | | | 2.7 | 2.8 | | 3.2 | 3.2 |
| Standard | | | 0.6 | 2.4 | | 1.2 | 0.6 |
| Deviation | | | | | | | |
| T-test against | | | | 37.4% | | | 100.0% |
| Comparative Plot | | | | | | | |
| (Probability | | | | | | | |
| according to t- | | | | | | | |
| distribution) | | | | | | | |
| | | Powderiness | | | Comprehensive Texture | | |
| | | | 80 Mesh | 270 Mesh | | 80 Mesh | 270 Mesh |
| | | | Comparative | Operation | | Comparative | Operation |
| | | Control | Plot | Plot | Control | Plot | Plot |
| Evaluator No. | 1 | 3.0 | 3.0 | 3.0 | 3.0 | 3.0 | 3.0 |
| | | (reference) | | | (reference) | | |
| | 2 | 3.0 | 3.0 | 3.0 | 3.0 | 2.5 | 3.0 |
| | | (reference) | | | (reference) | | |
| | 3 | 3.0 | 3.5 | 3.0 | 3.0 | 2.0 | 2.5 |
| | | (reference) | | | (reference) | | |
| | 4 | 3.0 | 3.5 | 3.5 | 3.0 | 2.5 | 2.5 |
| | | (reference) | | | (reference) | | |
| | 5 | 3.0 | 3.0 | 3.0 | 3.0 | 4.0 | 4.0 |
| | | (reference) | | | (reference) | | |

| | | | | |
|---|---|---|---|---|
| Average | 3.2 | 3.1 | 2.8 | 3.0 |
| Standard | 0.3 | 0.2 | 0.8 | 0.6 |
| Deviation | | | | |

| | | |
|---|---|---|
| T-test against | 37.4% | 17.8% |
| Comparative Plot | | |
| (Probability | | |
| according to t- | | |
| distribution) | | |

Results are shown in [Table 72]. As a result, in the item of hardness, as compared with the reference score of 3 of the control plot, there was a tendency of successively lowering from a score of 2.8 in the 270 mesh operation plot to a score of 2.7 in the 80 mesh comparative plot. In the item of viscoelasticity, as compared with the reference score of 3 of the control plot, the 270 mesh operation plot and the 80 mesh comparative plot had the same score of 3.2. In the item of powderiness, as compared with the reference score of 3 of the control plot, the 270 mesh operation plot had a score of 3.1 and the 80 mesh comparative plot had a score of a high value of 3.2. In the item of comprehensive texture, as compared with the reference score of 3 of the control plot, the 270 mesh operation plot had the same score and the 80 mesh comparative plot had a score of a low value of 2.8.

Based on these results, the 270 mesh operation plot had the scores closer to those of the control plot in almost all the items than the 80 mesh comparative plot.

[Measurement Method for Blood Glucose Level]

For the measurement of the blood glucose level, comparison was made between the 80 mesh comparative plot and the 270 mesh operation plot with the control plot excluded. The measurement of the blood glucose level was performed in Shimadaya Corporation, and subjects were 5 healthy males and females over 20 years old including a person having a fasting blood glucose level corresponding to borderline diabetes. For cooking, 150 g corresponding to each serving of each sample was boiled for 20 to 30 seconds in boiling water to be served after being dressed with 15 g of a commercially available pasta seasoning with no ingredients. Conditions were adjusted so that the pasta was able to be finished in 5 minutes after serving, and the blood glucose level was measured 30 minutes, 60 minutes, 90 minutes and 120 minutes after starting the ingestion.

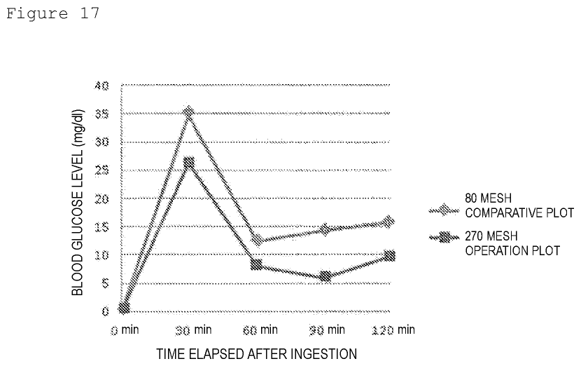

The thus obtained blood glucose level is shown in [Table 73], a change in the blood glucose level is shown in [Table 74], a difference (ΔCₘₐₓ) between a maximum blood glucose level and preprandial blood glucose level is shown in [Table 75], and a product (ΔAUC) of the blood glucose level and the time calculated using the preprandial blood glucose level as a baseline is shown in [Table 76]. Besides, transition of an average of the blood glucose levels is illustrated in a graph of FIG. 17.

TABLE 73
unit: mg/dl

| Time Elapsed | | 80 Mesh Comparative Plot | | | | | 270 Mesh Operation Plot | | | | |
|---|---|---|---|---|---|---|---|---|---|---|---|
| after Ingestion | | Prior | 30 min | 60 min | 90 min | 120 mm | Prior | 30 min | 60 min | 90 min | 120 mm |
| Subject | 1 | 96.0 | 134.0 | 107.5 | 107.0 | 109.0 | 96.0 | 117.5 | 107.0 | 102.0 | 103.5 |
| No | 2 | 89.5 | 117.0 | 98.5 | 102.0 | 118.5 | 97.5 | 116.5 | 97.0 | 98.5 | 106.5 |
| | 3 | 91.5 | 123.5 | 99.5 | 102.5 | 104.0 | 94.5 | 124.5 | 93.5 | 100.5 | 100.0 |
| | 4 | 91.0 | 131.0 | 109.5 | 104.5 | 102.5 | 98.5 | 126.0 | 119.0 | 99.5 | 109.0 |
| | 5 | 87.5 | 123.5 | 102.5 | 111.0 | 100.5 | 91.0 | 124.5 | 101.0 | 106.5 | 106.5 |

| | | | | | | | | | | |
|---|---|---|---|---|---|---|---|---|---|---|
| Average of Subjects | 91.1 | 125.8 | 103.5 | 105.4 | 106.9 | 95.5 | 121.8 | 103.5 | 101.4 | 105.1 |

| | | | | | |
|---|---|---|---|---|---|
| T-test against 80 Mesh Comparative Plot (Probability | 4% | 29.4% | 100% | 0% | 64% |
| according to t-distribution) | | | | | |

TABLE 74
unit: mg/dl

| Time Elapsed | | 80 Mesh Comparative Plot | | | | 270 Mesh Operation Plot | | | |
|---|---|---|---|---|---|---|---|---|---|
| after Ingestion | | 30 min | 60 min | 90 min | 120 min | 30 min | 60 min | 90 min | 120 min |
| Subject | 1 | 38.0 | 11.5 | 11.0 | 13.0 | 21.5 | 11.0 | 6.0 | 7.5 |
| No. | 2 | 27.5 | 9.0 | 12.5 | 29.0 | 19.0 | −0.5 | 1.0 | 9.0 |
| | 3 | 32.0 | 8.0 | 11.0 | 12.5 | 30.0 | −1.0 | 6.0 | 5.5 |
| | 4 | 40.0 | 18.5 | 13.5 | 11.5 | 27.5 | 20.5 | 1.0 | 10.5 |
| | 5 | 36.0 | 15.0 | 23.5 | 13.0 | 33.5 | 10.0 | 15.5 | 15.5 |

| | | | | | | | | |
|---|---|---|---|---|---|---|---|---|
| Average of Subjects | 34.7 | 12.4 | 14.3 | 15.8 | 26.3 | 8.0 | 5.9 | 9.6 |

| | | | | |
|---|---|---|---|---|
| T-test against 80 Mesh Comparative Plot (Probability | 4% | 13% | 1% | 18% |
| according to t-distribution) | | | | |

TABLE 75
unit: mg/dl

| | 80 Mesh | 270 Mesh |
|---|---|---|
| Δ Cmax | Comparative Plot | Operation Plot |

| | | | |
|---|---|---|---|
| Subject No. | 1 | 38.0 | 21.5 |
| | 2 | 29.0 | 19.0 |
| | 3 | 32.0 | 30.0 |
| | 4 | 40.0 | 27.5 |
| | 5 | 36.0 | 33.5 |

| | | |
|---|---|---|
| Average of Subjects | 35.0 | 26.3 |
| Standard Deviation | 4.5 | 6.0 |

| | |
|---|---|
| T-test against 80 Mesh Comparative Plot | 3.7% |
| (Probability according to t-distribution) | |

TABLE 76
unit: mg · min/dl

| | 80 Mesh | 270 Mesh |
|---|---|---|
| Δ AUC | Comparative Plot | Operation Plot |

| | | | |
|---|---|---|---|
| Subject No. | 1 | 2010 | 1268 |
| | 2 | 1905 | 720 |
| | 3 | 1718 | 1133 |
| | 4 | 2333 | 1628 |
| | 5 | 2430 | 2003 |

| | | |
|---|---|---|
| Average of Subjects | 2079 | 1350 |
| Standard Deviation | 297 | 488 |

| | |
|---|---|
| T-test against 80 Mesh Comparative Plot | 0.5% |
| (Probability according to t-distribution) | |

[Measurement Results of Blood Glucose Level]

An average of the preprandial blood glucose levels of the 5 subjects was 91.1 to 95.5 mg/dl, ΔCₘₐₓ was 35.0 mg/dl in the 80 mesh comparative plot, but was an obviously low value of 26.3 mg/dl in the 270 mesh operation plot, and a significant difference was found by a t-test. Besides, ΔAUC was 2079 mg·min/dl in the 80 mesh comparative plot, but was an obviously low value of 1350 mg·min/dl in the 270 mesh operation plot, and a significant difference was found by a t-test.

In other words, it was confirmed that the effect of suppressing the blood glucose level increase can be obtained also in frozen boiled pasta by using calcium alginate pulverized to 270 mesh pass as compared with that of 80 mesh pass.

### INDUSTRIAL APPLICABILITY

The present invention provides good-texture healthy boiled noodles retaining original texture of noodles having a health function of calcium alginate such as an effect of suppressing postprandial blood glucose level increase effectively retained, and in addition, without causing, in production of the noodles, deterioration of viscoelasticity and the like of noodle tissue and deterioration of texture otherwise caused in adding calcium alginate. When the method of the present invention is applied to production of boiled udon, boiled udon in which calcium alginate does not elute during a boiling step so that viscosity increase of hot water used for boiling can be restrained, in which deterioration of texture corresponding to a characteristic of udon, such as softness and chewy texture, can be prevented, and with which rapid increase of postprandial blood glucose level can be suppressed is provided.

1. A method for producing healthy boiled noodles having a health function of calcium alginate and retaining a good texture of noodles, wherein in a method for producing boiled noodles comprising (A) a mixing and kneading step of kneading a dough by adding kneading water to a powder raw material for noodle production obtained through a mixture of raw materials containing wheat flour; (B) a noodle making step of forming the dough into noodle strings; and (C) a boiling step of boiling and cooling the noodle strings to prepare boiled noodles, a 270-mesh pass pulverized calcium alginate powder is mixed in the powder raw material for noodle production in a ratio of 4 to 8 parts by mass based on 100 parts by mass of the powder raw material for noodle production.

2. The method for producing healthy boiled noodles according to claim 1, wherein the health function of the healthy boiled noodles is an effect of suppressing postprandial blood glucose level increase.

3. The method for producing healthy boiled noodles according to claim 1 or 2, wherein the boiled noodles are selected from boiled udon, chilled boiled buckwheat noodles, chilled boiled Chinese noodles and frozen boiled pasta.

4. A method for producing healthy raw noodles having a health function of calcium alginate and retaining a good texture of noodles, wherein in a method for producing raw noodles for boiled cooking comprising (A) a mixing and kneading step of kneading a dough by adding kneading water to a powder raw material for noodle production obtained through a mixture of raw materials containing wheat flour; (B) a noodle making step of forming the dough into noodle strings; and (C) a step of cutting the noodle strings into a predetermined amount, a 270-mesh pass pulverized calcium alginate powder is mixed, for production, in the powder raw material for noodle production in a ratio of 4 to 8 parts by mass based on 100 parts by mass of the powder raw material for noodle production.

5. The method for producing raw noodles of healthy raw noodles according to claim 4, wherein the raw noodles are selected from chilled raw udon, chilled raw buckwheat noodles, chilled raw pasta and chilled raw Chinese noodles.

6. A method for allowing boiled noodles or raw noodles to retain a good texture of noodles and for imparting a health function of calcium alginate thereto, wherein in a method for producing boiled noodles comprising (A) a mixing and kneading step of kneading a dough by adding kneading water to a powder raw material for noodle production obtained through a mixture of raw materials containing wheat flour; (B) a noodle making step of forming the dough into noodle strings; and (C) a boiling step of boiling and cooling the noodle strings to prepare boiled noodles, or in a method for producing raw noodles for boiled cooking comprising (A) a mixing and kneading step of kneading a dough by adding kneading water to a powder raw material for noodle production obtained through a mixture of raw materials containing wheat flour; (B) a noodle making step of forming the dough into noodle strings; and (C) a step of cutting the noodle strings into a predetermined amount, a 270-mesh pass pulverized calcium alginate powder is mixed in the powder raw material for noodle production in a ratio of 4 to 8 parts by mass based on 100 parts by mass of the powder raw material for noodle production.
